(19) United States
(12) Patent Application Publication
(10) Pub. No.: US 2009/0035827 A1
(43) Pub. Date: Feb. 5, 2009

(54) O-acetyltransferase from Neisseria Meningitidis, Compositions and Methods

(76) Inventors: David S. Stephens, Stone Mountain, GA (US); Charlene Kahler, Perth (AU)

Correspondence Address: GREENLEE WINNER AND SULLIVAN P C, 4875 PEARL EAST CIRCLE, SUITE 200, BOULDER, CO, 80301, US

(21) Appl. No.: 11/843,933

(22) Filed: Aug. 23, 2007

Related U.S. Application Data
(60) Provisional application No. 60/823,591, filed on Aug. 25, 2006.

(51) Int. Cl. C12P 19/04; C12N 1/21; C12N 9/10; C12N 15/54

(52) U.S. Cl. 435/101; 536/23.2; 435/252.3; 435/193

(57) ABSTRACT

Provided are recombinant DNA molecules that do not occur in nature encoding a Lot3 O-acetyltransferase, vectors that direct expression of a Lot O-acetyltransferase, recombinant host cells which express a Lot3 O-acetyltransferase, methods for recombinant production of a Lot3 O-acetyltransferase, methods for acetylating lipooligosaccharides, especially those of a Neisseria meningitidis using a recombinant Lot O-acetyltransferase, and immunogenic compositions comprising the acetylated lipooligosaccharide.

### CROSS-REFERENCE TO RELATED APPLICATIONS

This application claims benefit of U.S. Provisional Application 60/823,591, filed Aug. 25, 2006, which application is incorporated by reference herein to the extent there is no inconsistency with the present disclosure.

### STATEMENT REGARDING FEDERALLY SPONSORED RESEARCH OR DEVELOPMENT

This invention was made with government support under United States Public Health Service Grant R01 AI033517 awarded by the National Institutes of Health. The United States Government has certain rights in the invention.

### BACKGROUND OF THE INVENTION

The field of this invention is molecular biology, in particular as applied to Neisseria meningitidis, as well as the preparation of immunogenic compositions comprising lipooligosaccharides and/or protein from N. meningitidis.

Neisseria meningitidis is the causative agent of epidemic meningitis and fatal septic shock (1). Being an obligate human pathogen, the human nasopharynx is the only known natural environmental niche from which the organism spreads to other human hosts via inhalation of respiratory droplets. In circumstances which are not entirely understood, nasopharyngeal acquisition may also result in invasive meningococcal disease (1, 2). Comparative studies of invasive and carriage isolates of meningococci implicate lipooligosaccharide (LOS) in the attachment and invasion of the nasopharyngeal epithelium (3, 4) and in the pathogenesis of invasive meningococcal disease (5). Meningococcal isolates express 12 immunologically distinct LOS structures (L1-L12) (6-8) which were subsequently shown to correspond to distinct chemical structures (9-13). Examination of carriage and invasive disease isolates by serology indicates that meningococci commonly co-express patterns of LOS immunotypes, with L[1,8], L[3,7,9] and L[2,4] being the most prevalent (3, 4).

Each LOS immunotype structure has a conserved heptose inner core to which α-, β- and γ-chains are added (FIG. 1). The length and composition of the α-chain is based upon the phase variable expression of IgtA, IgtD and IgtC (for reviews see 14, 15). The length and composition of the α- and β-chain extensions from HepI as well as the presence or absence of a γ-chain extension and phosphoethanolamine residues (PEA) on HepII appear to determine immunotype. The presence of the γ-chain extension of α1-3 glucose on HepII, characteristic of L2 and L5 immunotype LOS (FIG. 1), is determined by the phase variable expression of IgtG (16). In comparison, the presence or absence of the various PEA groups on the inner core is in part determined by whether or not the isolate carries an intact Ipt3 or Ipt6 gene (17, 18). In some meningococcal strains, O-3 linked PEA is attached to HepII by Lpt3 in the absence of LgtG activity, thus producing the L3 immunotype (17). However, an O-3-linked PEA could not be demonstrated in strain NMB which expresses L[2,4] immunotype LOS and contains an intact Ipt3(18). Ram et al (19) have proposed that PEA variable additions to the inner core play an important role in protecting meningococci from complement mediated lysis, since the O-6 linked PEA groups of L2 and L4 LOS inner core and the exposed O-3 linked PEA groups of L[1,8] LOS are targets for complement component C4b. Mechanisms that mask these PEA inner core-associated epitopes, such as the co-expression of a long α-chain, or prevention of the expression of PEA groups on the cell surface, such as competition by the glucosyltransferase, LgtG (17), increase the resistance to complement mediated lysis (19).

Recently, it has become clear that at least some strains of meningococci have the capacity to express all immunotype LOS structures and that complex regulatory networks and structural constraints dictate what pattern of immunotypes are expressed during growth (18). Comparison of the meningococcal LOS inner core structures of different immunotypes reveals that structures without O-3 linked PEA groups are invariably O-acetylated (FIG. 1). We hypothesized that O-acetylation of the terminal LOS inner core N-acetylglucosamine in the presence of a lactoneotetraose α-chain could prevent the enzymatic addition of O-3 linked PEA by Lpt-3 to this substrate. To test this hypothesis, the lipooligosaccharide O-acetyltransferase (lot3) was identified in N. meningitidis strain Z2491 by amino acid homology with a known O-acetyltransferase, from Rhizobium leguminosarum (20).

Insertional inactivation of lot3 in N. meningitidis strain NMB confirmed that this locus encoded the lipooligosaccharide O-acetyltransferase. In addition, inactivation of both lot3 and IgtG resulted in the appearance of O-3 linked PEA on the LOS inner core of this strain. Therefore, O-acetylation of the terminal N-acetylglucosamine residue of the LOS inner core when expressed in combination with a lactoneotetraose α chain physically prevents the addition of O-3 linked PEA groups to this site by a functional Lpt-3.

### BRIEF SUMMARY

Provided herein are recombinant DNA molecules which do not occur in nature, recombinant host cells and methods of using the foregoing to recombinantly produce a lot O-acetyltransferase derived from Neisseria meningitidis or an equivalent O-acetyltransferase from another species of Neisseria. Specifically exemplified amino acid sequences are given in SEQ ID NO: 5-16 and 34, and coding sequences are given in SEQ ID NOs: 17-33. Such an acetyltransferase transfers acetyl moieties to lipooligosaccharides, especially those of N. meningitidis. The O-acetyltransferase can be purified using specific antibody in an immunoaffinity column, for example, or an affinity tag can be engineered into the recombinant protein by the use of appropriate tag (especially a polyhistidine or His tag) coding sequences fused in frame. Other oligopeptide “tags” which can be fused to a protein of interest by such techniques include, without limitation, strep-tag (Sigma-Genosys, The Woodlands, Tex.) which directs binding to streptavidin or its derivative streptactin (Sigma-Genosys); a glutathione-S-transferase gene fusion system which directs binding to glutathione coupled to a solid support (Amersham Pharmacia Biotech, Uppsala, Sweden); a calmodulin-binding peptide fusion system which allows purification using a calmodulin resin (Stratagene, La Jolla, Calif.); a maltose binding protein fusion system allowing binding to an amylose resin (New England Biolabs, Beverly, Mass.); and an oligo-histidine fusion peptide system which allows purification using a Ni²⁺-NTA column (Qiagen, Valencia, Calif.).

Further encompassed by the present disclosure is the acetylation (in vitro) of lipooligosaccharides isolated from N. meningitidis or other species of Neisseria using acetyltransferase recombinantly produced using the recombinant host cells taught herein.

Also provided herein are cells of N. meningitidis or other Neisseria species into which a functional, expressible lot gene is overexpressed, for example by inserting a second active copy of this sequence, or by expressing a lot3 coding sequence operably linked to a strong promoter in a Lot3 genetic background, either strategy having the result that there is a greater expression of Lot3 protein than in a wild type strain. Such strains produce highly (fully) acetylated lipooligosaccharide. Such LOS is advantageously formulated into immunogenic compositions, for example those destined for human vaccines. The highly acetylated LOS can be combined with other neisserial antigens, including capsular polysaccharides and/or other cellular components. Immunization with a composition of the present invention results in improved protection of a human exposed to a pathogenic Neisseria, especially N. meningitidis.

In addition, there are provided improved immunogenic compositions comprising oligosaccharides and lipooligosaccharides of N. meningitidis, or other Neisserial species, where the improvement comprises more complete acetylation of the saccharides than is currently possible in the absence of the enzymatic acetylation by using (in vivo or in vitro) the acetyltransferase of the present invention, especially those from N. meningitidis, with the result that a stronger immune response and greater protection against infection, results. The immunogenic compositions of the present invention can comprise a pharmaceutically acceptable carrier and optionally can further comprise at least one immunological adjuvant or cytokine. These immunogenic compositions are useful as vaccines and as vaccine components. Also encompassed are non-acetylated oligosaccharides and lipooligosaccharides from Neisseria, especially produced by a lot mutant of N. meningitidis or other species of Neisseria.

It is a further object to provide nonacetylated OS and/or LOS for use in assay kits, for example, diagnostic assay kits. Such kits can be employed with acetylated and nonacetylated LOS for distinguishing N. meningitidis producing different immunotypes of LOS, for example. Similar assays and kits can be prepared to application to other species of Neisseria which produce such an acetyltransferase, using the OS and/or LOS from the cognate species where it is desired to learn whether acetylated saccharides are produced.

### BRIEF DESCRIPTION OF THE DRAWINGS

FIG. 1 shows meningococcal LOS structures of L1-L6 and L8 immunotype strains (9-13). The L7 immunotype structure (not shown) is the non-sialylated version of the L3 immunotype structure (11). The conserved inner core region is shown with variable attachments denoted as R1-R5. The composition of the α chain (R1) is governed by the phase variable expression of the IgtA-E transferases (15) and Ist, which encodes the sialyltransferase that attaches the terminal α-Neu5Ac (sialic acid) group. Note that attachment of glycine to the inner cores of L1 and L5-7 immunotypes have not been investigated using current techniques. This figure is modified from Kogan et al. (11).

FIG. 2 illustrates organization of the lot3 locus (NMA2202) in N. meningitidis strain Z2491. The location of the primers (small arrows) and restriction site (SmaI) used during mutagenesis are indicated. NMA2201 and NMA2204 encode hypothetical proteins, whereas NMA2203 encodes a putative adenylosuccinate lyase (purB) and NMA2205 encodes a putative ribonuclease 11 related protein.

FIGS. 3A-3C provide the positive MALDI-TOF MS spectra of the major NMB parent OS structure (FIG. 3A), the major OS from NMBlot3 (FIG. 3B), and the major OS from NMBlot3/IgtG (FIG. 3C). The ions and proposed structures for these ions are as indicated.

FIG. 4 shows the ¹H-¹H TOCSY NMR of the major OS isolated from NMB wild type LOS. The TOCSY correlations are indicated by the dotted lines. Other correlations not indicated in this figure are given in Table 2.

FIGS. 5A-5C provide ¹H-¹³C gHSQC NMR spectra of the OS from (FIG. 5A) NMB LOS, (FIG. 5B) NMBlot3, and from (FIG. 5C) NMBlot3/IgtG LOS. The anomeric H/C signals are labeled from A to H for the glycosyl residues shown for these OS structures in FIG. 3, and the assignments in Tables 1 and 2. Resonances not indicated in this figure are also given in Tables 1 and 2.

FIGS. 6A-6B show the ³¹P-¹H HMQC spectra of the major OS from NMBlot3 (FIG. 6A) and the major OS from NMBlot3/IgtG (FIG. 6B). The assignments of the resonances are as indicated.

FIGS. 7A-7B show the ³¹P-¹H HMQC-TOCSY spectra of the major OS from NMBlot3 (FIG. 7A), and the major OS from NMBlot3/IgtG (FIG. 7B). The assignments of the resonances are as indicated.

### DETAILED DESCRIPTION OF THE INVENTION

The abbreviations used herein are OS, oligosaccharide; LOS, lipooligosaccharide; CPS, capsular polysaccharide; O—Ac CPS, O-acetylated capsular polysaccharide; PCR, polymerase chain reaction; GC-MS, gas-liquid chromatography-mass spectrometry; COSY, ¹H-¹H correlation spectroscopy; TOCSY, total correlation spectroscopy; mAb, monoclonal antibody; ELISA: enzyme linked immunosorbant assay; SDS, sodium dodecyl sulfate; Hep, heptose; PEA, phosphatidyl ethanolamine.

O-acetylation is a common decoration on endotoxins derived from many Gram-negative bacterial species, and has been shown to be instrumental (e.g. Salmonella typhimurium) in determining the final tertiary structure of the endotoxin, and the immunogenicity of the molecule. Structural heterogeneity of endotoxins produced by mucosal pathogens such as N. meningitidis is determined by decorations on the heptose inner core, including O-acetylation of the terminal N-acetylglucosamine (GlcNAc) attached to HepII. In this report, we show that O-acetylation of the meningococcal LOS inner core has an important role in determining inner core assembly and immunotype expression. The gene encoding the lipooligosaccharide O-acetyltransferase, lot3, was identified by homology to NodX from Rhizobium leguminosarum. Inactivation of lot3 in strain NMB resulted in the loss of the O-acetyl group located at the C-3 position of the terminal GlcNAc of the LOS inner core. While inactivation of lot3 or IgtG encoding the HepII glucosyltransferase alone did not result in the appearance of the O-3 linked PEA groups on the LOS inner core, construction of a double mutant in which both lot3 and IgtG were inactivated resulted in the appearance of O-3 linked PEA groups. In conclusion, O-acetylation status of the terminal GlcNAc of the γ-chain of the meningococcal LOS inner core is an important determinant for the appearance or exclusion of the O-3 linked PEA group on the LOS inner core, and contributes to LOS structural diversity. O-acetylation also likely influences resistance to complement mediated lysis and may be important in LOS conjugate vaccine design.

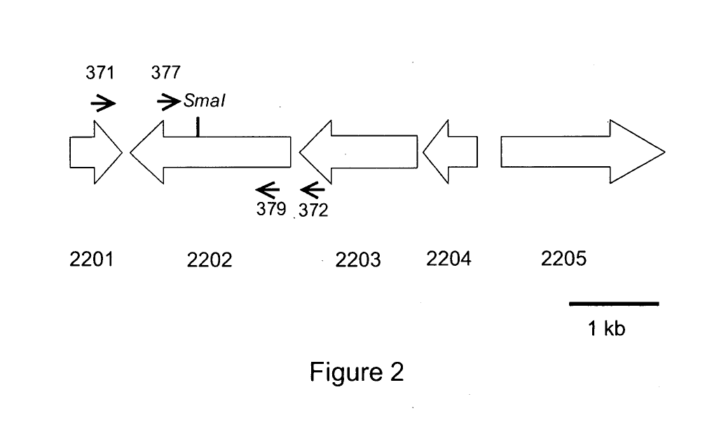

This specification describes the identification of the lipooligosaccharide C-3 O-acetyl transferase gene (lot3) in N. meningitidis strain NMB. Meningococcal LOS immunotypes L2 and L4 are O-acetylated at the C-3 position of the non-reducing terminal α-N-acetyl-D-glucosamine (GlcNAc) (11). Rhizobium leguminosarum biovar viciae expresses lipooligosaccharide nodulation factors containing oligomers of four to five beta 1-4 linked GlcNAc residues with the reducing-end GlcNAc O-acetylated by NodX at the C-6 position (33). A BlastP search of the complete peptide databases of N. meningitidis strain MC58 and strain Z2491 using the amino acid sequence of NodX from R. leguminosarum biovar viviae strain TOM (34) revealed two potential candidate LOS O-acetyltransferases (FIG. 2). NMA0619 encodes a 622 amino acid peptide with 30% identity over the first 191 amino acids of NodX. However, the corresponding open reading frame in N. meningitidis strain MC58 (Serogroup B), NMB1836, is intact, and has recently been shown to encode an acyltransferase (PgII) required for the biosynthesis of the basal 2,4-diacetamido-2,4,6-trideoxyhexose residue of the pilin-linked glycan (35). The second candidate, NMA2202, was present as an intact open reading frame in strain Z2491 (Serogroup A) which expresses an L9 LOS (on the internet/worldwide web, address sanger.ac.uk/Projects/N_meningitidis/seroA/strain.shtml) and had 30% amino acid identity over the first 154 amino acids of NodX. In strain MC58, the ORF corresponding to NMA2202, NMB2032, contains a 6 bp poly-G tract which causes a translational frameshift. To further determine whether there was a relationship between an intact NMA2202 open reading frame and O-acetylation of LOS, we examined the locus in strain NMB which expresses an L2 LOS (36). Like strain Z2491, strain NMB contained an intact NMA2202 open reading frame containing a 5 bp poly-G tract.

NMA2202 encodes the lipooligosaccharide O-acetyltransferase (Lot3). A mutant in which NMA2202 of strain NMB was insertionally inactivated by a tetM antibiotic resistance cassette was constructed and designated NMBlot3 (see below). LOS was extracted and the oligosaccharides (OS) were prepared as described below. Purification of the OSs by gel-filtration chromatography using Bio-Gel P2 resulted in one major and two minor OS peaks from NMBlot3. Glycosyl composition and linkage analysis of the major OS from NMBlot3 showed the glycosyl residues and linkages expected from the published NMB LOS structure (36); namely, terminally linked glucose (Glc), terminally linked galactose (Gal), terminally linked N-acetylglucosamine (GlcNAc), 4-linked Glc, 3-linked Gal, 4-linked GlcNAc, and 3,4-linked heptosyl (Hep) I. The Hep II residue was not observed since phosphorylated residues are not detected in the method used and Hep II is the location of phosphoethanolamine (PEA) groups. The minor OSs from NMBlot3 were also analyzed by glycosyl composition analysis and mass spectrometry. The results showed that the minor OSs consisted of glycinylated versions of the major OS structure (i.e. the ion masses were 57 mass units greater than those of the major OS), as well as the presence of detectable amounts of sialic acid monosaccharide that was presumably released from the α-chain during mild acid hydrolysis.

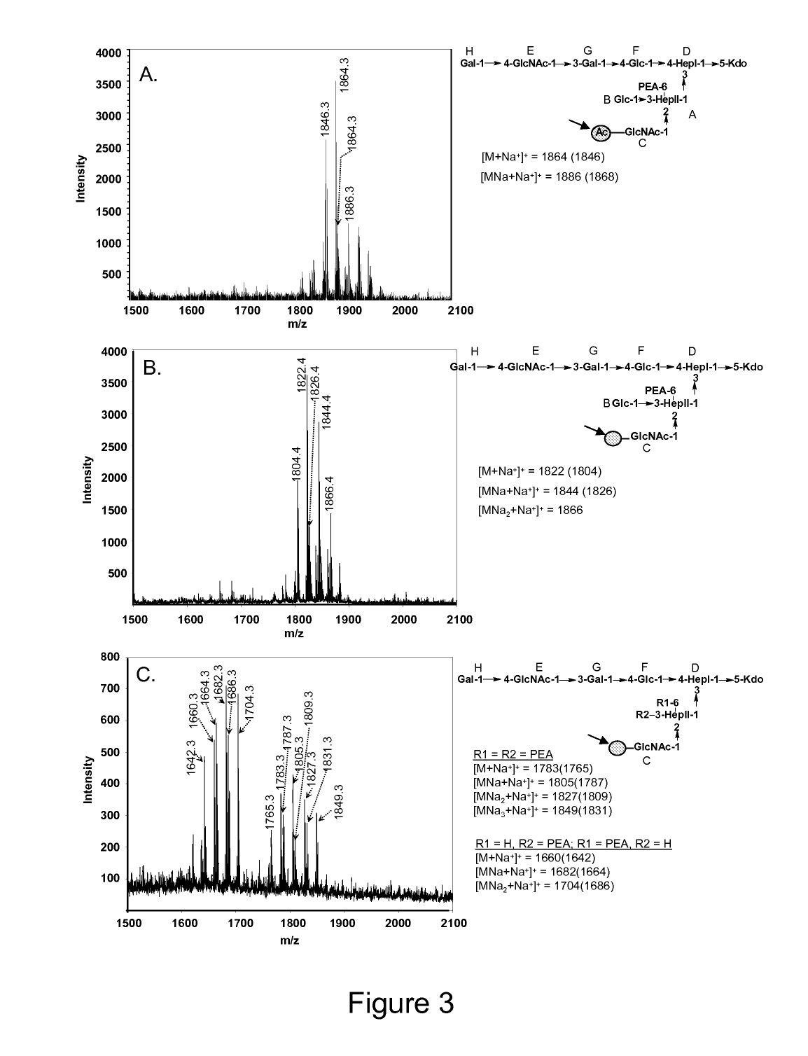

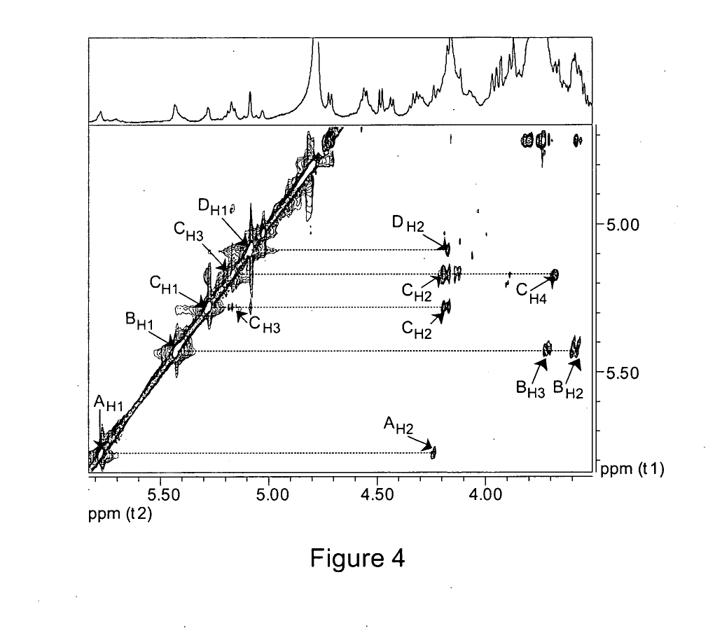

The major OSs from the LOSs of NMB, and NMBlot3 were then analyzed by MALDI-TOF MS and the resulting spectra are shown in FIGS. 3A and 3B, respectively. The molecular ions observed for the OS from NMBlot3 (m/z 1804, 1822, 1826, 1844, and 1866; FIG. 3B) are all consistent with sodiated and anhydro-sodiated forms of a single oligosaccharide structure. These masses are 42 mass units less than the corresponding ion masses for the OS from NMB, FIG. 3A. This result is consistent with the NMBlot3 LOS having a structure that is the same as reported for NMB LOS (36) with the notable exception that the NMBlot3 OS lacks an acetyl group. Previous work on the NMB LOS had reported that the OS was acetylated at O-6 of the GlcNAc residue terminally linked to O-2 of Hep II (36). This conclusion was based on comparison of 1-D TOCSY NMR spectra of the NMB OS taken before and after HF treatment which removes PEA as well as the O-acetyl group from the OS. This work on the NMBlot3 LOS required that we re-examine the location of this O-acetyl group in the NMB LOS, as well as confirm its absence in the NMBlot3 LOS. This was accomplished by examining the OS from NMB and from NMBlot3 by gradient correlation spectroscopy (gCOSY), total correlation spectroscopy (TOCSY), and heteronuclear single quantum coherence spectroscopy (gHSQC). From these experiments, it was possible to make assignments for the majority of the protons for both the NMB OS (Table 3) and the NMBlot3 OS (Table 2). Comparison of the assignments for these OSs showed that the α-GlcNAc proton resonances for the NMB OS were quite different from those for the NMBlot3 OS. In particular, the H3 of this α-GlcNAc residue in NMB OS resonates at δ 5.17, which is considerably downfield from the H3 resonance of this residue in NMBlot3 OS which resonates at δ 3.85. This downfield shift is consistent with O-acetylation at this position in NMB OS and a lack of this acetylation in the NMBlot3 OS. A TOCSY spectrum of the NMB OS showing the coupling of the H1 (δ 5.28), H2 (δ 4.19), H3 (δ 5.17), and H4 (δ 3.68) protons of the α-GlcNAc residue is given in FIG. 4.

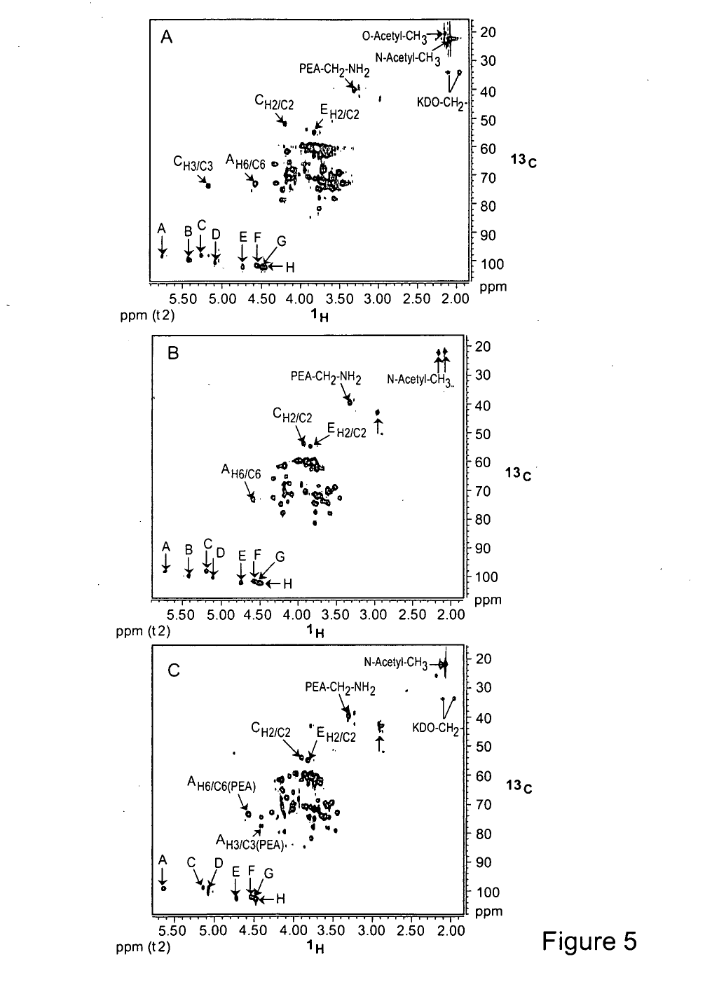

The presence and location, or absence of O-acetyl groups can also be observed in the gHSQC spectra of the NMB and NMBlot3 OS samples; FIGS. 5A and 5B, respectively. The NMB OS gHSQC spectrum (FIG. 5A) shows two N-acetyl methyl protons (carbons) at δ 2.04 and 2.06 (δ 22.5), and the O-acetyl methyl protons (carbon) at δ 2.11 (δ 20.6). However, only two N-acetyl methyl protons (carbons) are observed in the NMBlot3 OS spectrum (FIG. 5B) at δ 2.11 (a 22.5) and 2.04 (δ 21.9). The NMBlot3 OS lacks the O-acetyl resonances. The gHSQC spectrum of the NMB OS also contributed to assigning the location of the O-acetyl group to O-3 of the α-linked GlcNAc residue. From the gCOSY spectrum (not shown), the anomeric proton of the α-GlcNAc residue (δ 5.28) was shown to be coupled to H2 at δ 4.19. The gHSQC spectrum (FIG. 5A) shows that this H2 is coupled to the C2 at δ C 51.9 which confirms that this is the α-GlcNAc residue. These data, together with the TOCSY data (see FIG. 4) allowed the assignment of the resonances for the GlcNAc residue that are given in Table 2. From these assignments, it was shown that the H3 of this GlcNAc is at γ 5.17 and is coupled to a carbon at δ C 74.0 (see FIG. 5A). This H3/C3 coupling is clearly absent in the gHSQC spectrum of the NMBlot3 OS, FIG. 5B. Thus, these NMR data support the mass spectrometric data which show that the OS from NMBlot3 LOS lacks an O-acetyl group, and that the O-acetyl group on NMB LOS is located at O-3 of the α-GlcNAc residue and not at O-6 as previously reported (36).

O-acetylation of the terminal GlcNAc residue of meningococcal LOS influences PEA additions to the inner core. The meningococcal LOS inner core structure is masked by the additions to HepII which are highly variable and heterogeneous (14). One of these additions, the presence of the γ-chain extension of α1-3 glucose on HepII, characteristic of L2 and L5 immunotype meningococcal LOS, is determined by the phase variable expression of IgtG (16). In some meningococcal strains, O-3 linked PEA is attached to HepII by Lpt3 in the absence of LgtG activity, thus producing the L3 immunotype (17). However, in strain NMB which contains an intact Lpt3, inactivation of IgtG did not result in the appearance of O-3 linked PEA on the inner core of the LOS (18). Conversely, O-3 linked PEA is an inner core decoration in mutants of strain NMB which lacked the addition of the α-chain (18, 37, 38). These results suggested that the conformation adopted by the inner core with the addition of the α-chain, in combination with the O-acetylation of the terminal GlcNAc residue, precluded the addition of O-3 PEA groups by Lpt3. To test this important hypothesis which has implications for immunotype switching in vivo, a mutant strain in which both IgtG and lot3 were insertionally inactivated, designated NMBlot3/IgtG, was analyzed for the appearance of O-3 PEA groups on the inner core in the presence of an intact α-chain. Purification of the OSs by gel-filtration chromatography using Bio-Gel P2 resulted in one major and additional minor OSs from NMBlot3/IgtG. The major OS from NMBlot3/IgtG had the same glycosyl residue linkages as those for NMBlot3 OS with the exception that terminally linked Glc was absent; a result which is consistent with the mutation in IgtG.

The major OSs were analyzed by MALDI-TOF MS and the resulting spectrum is shown in FIG. 3C. The mass spectrum of the OS from NMBlot3/IgtG, shows two clusters of ions. Each cluster represents a single structure. The ions of the first cluster (m/z 1765, 1783, 1787, 1805, 1809, 1827, 1831, and 1849) are all consistent with various sodiated and anhydro sodiated forms of a structure that, compared to the NMBlot3 OS structure, lacks one hexosyl residue and contains an additional (i.e. for a total of two) PEA group. The second cluster ions (m/z 1642, 1660, 1664, 1682, 1686, and 1704) are consistent with sodiated and anhydro sodiated forms of a structure that, compared to the NMBlot3 OS structure, lacks a hexosyl residue. This missing hexosyl residue is consistent with the methylation data showing that this OS lacks a terminally linked Glc; i.e. the terminal Glc that is linked to O-3 of Hep II. In addition to the glycosyl linkage and MS data showing that the NMBlot3/IgtG OS lacks both the terminal α-Glc residue and the O-acetyl group, the lack of both these moieties was also confirmed by NMR analysis. Comparison with the spectra for NMB and NMBlot3 OSs (FIGS. 5A and 5B, respectively), the spectrum for the NMBlot3/IgtG OS (FIG. 5C) lacks the anomeric H/C signal for the α-Glc residue (residue B), and lacks the downfield H3/C3 resonance for the terminal α-GlcNAc as observed for the NMB OS (FIG. 5A). As observed for NMBlot3, the minor OSs from NMBlot3/IgtG consisted of glycinylated versions of the major OS. These results support the conclusions that the NMBlot3/IgtG mutant, which in addition to missing the O-acetyl group, lacks the terminally linked Glc attached to O-3 at Hep II, was able to express a major structure containing two PEA groups attached to Hep II.

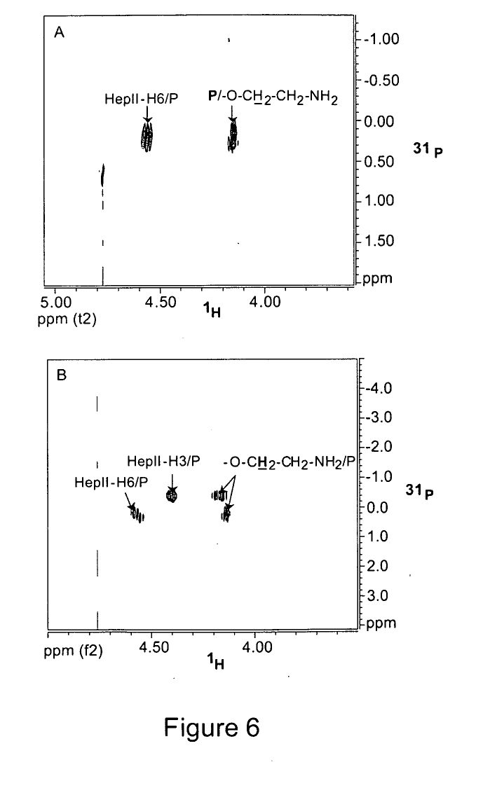

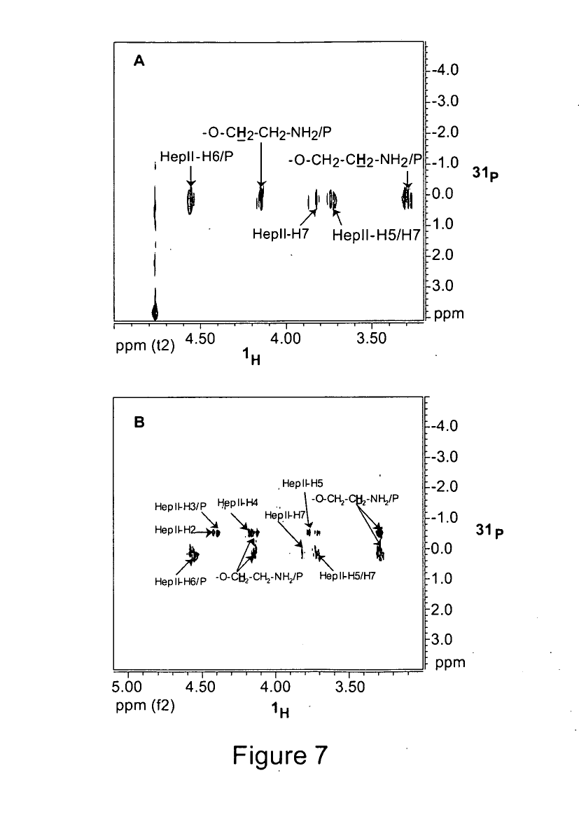

The location of LOS inner core PEA groups is influenced by O-acetylation. In order to determine the location of the PEA groups on NMBlot3 and NMBlot3/IgtG OSs, two NMR experiments were performed. The first was a ³¹P/¹H HMQC experiment and the spectra for the NMBlot3 and NMBlot3/IgtG OSs are shown in FIGS. 6A-6B. FIG. 6A shows that the phosphorous atom of the PEA group was correlated to the H-6 proton of Hep II and to the —OCH₂— methylene protons of the PEA group. These results were consistent with NMBlot3 OS containing a single PEA group attached to O-6 of Hep II. The spectrum of NMBlot3/IgtG OS shown in FIG. 6B was consistent with an OS that contains two PEA groups. For one PEA group the phosphorous atom was correlated to H-6 of Hep II and to —OCH₂— of PEA, and for the second PEA group the phosphorous atom was correlated to H-3 of Hep II and to —OCH₂— of PEA. These results are consistent with the NMBlot3/IgtG OS containing two PEA groups, at O-6 and O-3 of the Hep II residue. The locations of the PEA groups were further confirmed by ³¹P/¹H HMQC-TOCSY analysis. The spectrum for the NMBlot3 OS is shown in FIG. 7A and shows that the phosphorus of the PEA group is correlated to H-6, H-5, H-7a and H-7b of Hep II, and to the —O—CH₂— methylene and —CH2-NH2 methylene protons of the PEA group. The spectrum for the NMBlot3/IgtG OS, FIG. 7B, showed that the phosphorus of one PEA group was correlated with Hep II and PEA methylene protons consistent with a location at O-6 of Hep II as just described for the NMBlot3 OS, while the phosphorus of the second PEA group was correlated to H-3, H-2, H-4, and H-5 of Hep II and to the protons of both of the PEA methylene groups. These results confirm that the major OS from NMBlot3/IgtG LOS contains structures with two PEA groups located at O-3 and O-6 of Hep II, as well as with a single PEA group located at O-3 or O-6 of Hep II. Thus, in strain NMB, O-3 linked PEA groups appear on the LOS inner core only in the absence of both O-3 linked glucose at HepII and O-acetylation of the terminal GlcNAc. This is consistent with the hypothesis that in the presence of a full length α-chain and O-acetylation of terminal GlcNAc of the inner core, Lpt3 is unable to gain access to HepII of the inner core.

Lipooligosaccharide structure and variability in N. meningitidis has been shown to be important for the pathogenesis of this organism. An examination of various LOS immunotyped meningococcal strains by Jennings et al (15) demonstrated that one mechanism of LOS phase variation was due to changes in the length of homopolymeric tract regions of IgtA, IgtC and IgtG (Table 1). Interestingly, the most frequently detected reversible LOS immunotype switching event has been that of L[1,8] to L[3,7,9] since many isolates express a mixture of these structures (3, 4). Although immunotype switching involving L2 and L4 immunotypes is less frequent, it does occur with two natural events of L[1,2] (4) and L[2,3] (15) immunotype interconversions being described. Our study indicates that the LOS structural changes resulting in conversion between immunotype pattern expression of L[2,4] and L[3,7,9] LOS is in part dependent upon the phase variable expression of the lipooligosaccharide O-acetyltransferase encoded by lot3.

An L3 immunotype is distinguished from an L2 LOS immunotype by four structural changes to the HepII of the inner core: the loss or exclusion of O-3 linked PEA group from HepII, the addition of O-3 linked glucose by LgtG, the addition of O-6 linked PEA by Lpt6 and as shown in this study, the addition of the O-acetyl group to the terminal N-acetylglucosamine (GlcNAc). Mackinnon et al (17) demonstrated that expression of LgtG was sufficient to exclude the addition of O-3 linked PEA groups from the LOS inner core of L3 immunotype strains and ascribed this dominant effect to increased efficiency for this site by LgtG. However, inactivation of LgtG and Lpt6 in strain NMB, which expresses a mixture of L2 and L4 LOS immunotypes in a 5 to 1 ratio, does not result in the appearance of O-3 linked PEA groups on the inner core even though the strain contains an active Lpt3 (18). Mutations which removed the lactoneotetraose α-chain do result in the appearance of an O-3 linked PEA on LOS inner cores in strain NMB (18, 37, 38). From these data we concluded that the three dimensional structure of the LOS inner core with an intact α chain masks Hep II making the O-3 attachment site unavailable to Lpt-3. The only structural difference between the L3 LOS structure and that of strain NMB without O-3 linked glucose or O-6 linked PEA groups is the presence of an O-acetyl group on the terminal GlcNAc of the γ-chain (36).

The N. meningitidis lipooligosaccharide O-acetyltransferase (lot3) was identified by homology to a known LOS O-acetyltransferase from R. leguminosarum. Inactivation of lot3 in strain NMB resulted in the loss of the O-acetyl group located on the terminal GlcNAc of the LOS inner core. We found that O-acetylation of the GlcNAc residue is at O-3 as previously assigned by Kogan et al (11). The proton spectrum of the NMB OS in a previous report (36) clearly shows that it is identical to that obtained for our current analysis of NMB OS, and has the downfield α-GlcNAc H3 resonance at δ 5.17 due to O-acetylation at that position. The O-6 assignment in the earlier report was made by comparing 1-D TOCSY spectra before and after HF treatment of the OS. The fact that this treatment removes both O-acetyl and PEA groups and the rather poor quality of the 1-D TOCSY spectra resulted in the incorrect assignment of the O-acetyl group. Therefore, the lot3 locus in strain NMB has been designated lot3 indicating the linkage of the O-acetyl group at the O-3 position of the terminal GlcNAc residue in the L2 immunotype structure.

O-3 linked PEA groups did not appear on the LOS inner core of NMBlot3 mutants due to the dominant effect by LgtG as observed by Mackinnon (17). Inactivation of IgtG in the absence of Lot3 activity in strain NMB resulted in the appearance of O-3 linked PEA groups on the inner core, supporting the hypothesis that the O-acetyl group was responsible for obstructing access of Lpt-3 to the LOS inner core in the absence of O-3 linked glucose. Based upon previous observations on the effect of O-acetylation on the conformation of Salmonella typhimurium O-antigen (39) the most likely mechanism appears to be that the meningococcal LOS undergoes a conformational change with the addition of the O-3 acetylation of GlcNAc, which in the presence of the lactoneotetraose α chain, prevents access to the inner core by Lpt3. A comparison of lot3 genes found in the meningococcal genome databases with that of strain NMB indicates that a short polymeric G-tract is present in the central region of the open reading frame. The point mutation inactivating lot3 in strain MC58 which expressed L3 immunotype LOS was found in this region, however, whether the length of the polymeric tract undergoes phase variation at a detectable rate remains unknown.

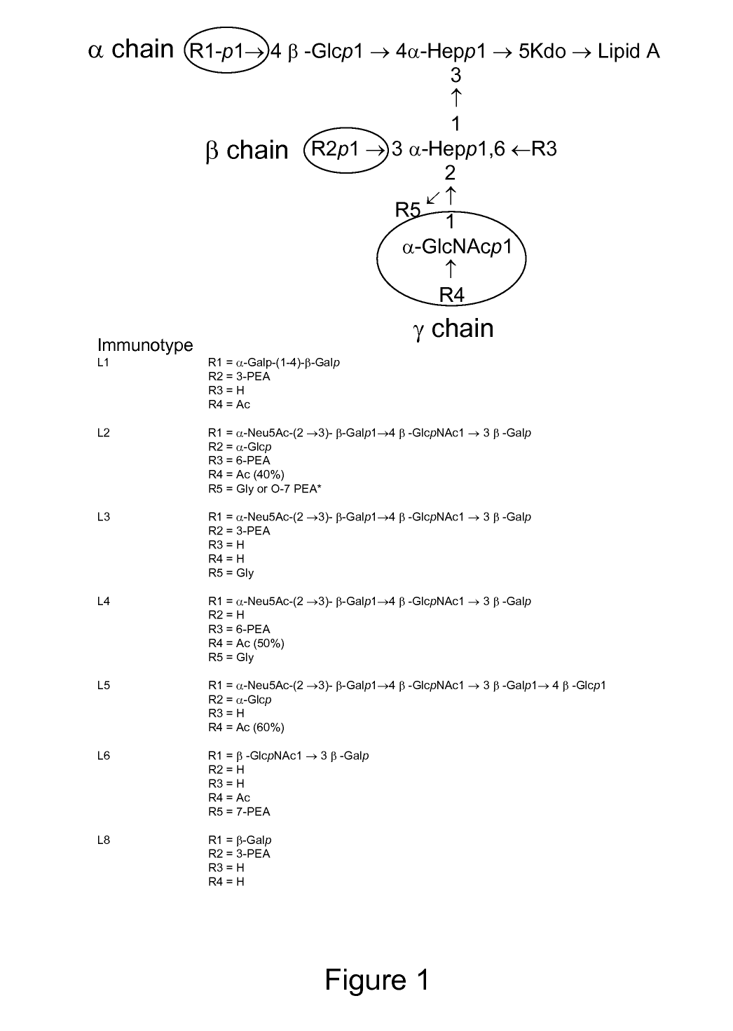

The lot3 locus has been found in all meningococcal strains examined to date, including isolates expressing all twelve immunotype structures. The intact lot3 gene was found in those strains expressing immunotype LOS structures with an O-acetyl group (FIG. 1, Table 1). Based on these data we believe that immunotypes L9-12 are also O-acetylated, indicating that this substitution is common amongst all immunotypes with the exception of L3 and L7. Based upon the structures for LOS immunotypes L1-8, and the data derived from mutating the LOS biosynthesis pathways in strains NMB and MC58, the following conclusions can be drawn regarding the expression of the O-3 PEA group on the LOS inner core of meningococcal strains. In meningococcal isolates expressing a LOS structure with a short α-chain (such as L1 and L8), O-3 PEA groups can be added to the inner core by Lpt-3 regardless of the O-acetylation state of the terminal GlcNAc of the γ-chain (18). In comparison, the spectrum of decorations added to the LOS inner core of meningococcal isolates expressing an α-chain consisting of lactoneotetraose is determined by the O-acetylation status of the terminal GlcNAc. When lot3 is not expressed in the presence of a lactoneotetraose α chain, the phase variable expression of IgtG and the presence or absence of functional Ipt3 and Ipt6 determines which decorations are added to the inner core (Table 1). When lot3 is expressed in the presence of a LOS structure with a full-length α-chain, even if the strain contains an active Ipt-3, only the O-6 linked PEA groups and O-3 linked glucose residues can be added to the LOS inner core. We cannot completely exclude the possibility that the absence of Lot may affect the stability or function of Lpt-3. However, it appears unlikely that Lot and Lpt-3 form a complex because the predicted structures of these proteins indicate that they reside in separate cell compartments (cytoplasm and periplasm, respectively).

The biological relevance of these findings is of considerable interest. Ram et al (19) have suggested that there is a strong positive selection pressure for the expression in invasive meningococcal disease of LOS structures with a lactoneotetraose chain and an inner core decorated with an O-3 PEA group, because this structure is unavailable for binding to the complement factor C4b. The O-3 PEA group results in increased resistance to killing by human serum during bloodstream dissemination. The identification of lot3 and the effect on the exclusion of the addition of O-3 PEA groups to the LOS inner core also suggest that there may be biological selection for preventing or masking the appearance of O-3 linked PEA groups on the surface of meningococci. LOS functions as an adherence molecule and cell invasion ligand (3,4,40,41), and LOS structure influences these events by obstruction of Opa adhesins (42). Alternatively, the effect on the presence of O-3 PEA groups may be an indirect consequence of immunological selection directed at the O-acetyl group on the LOS inner core. Investigations of the immunological responses generated by meningococcal LOS immunotypes have shown that the O-acetylated L2 and L5 structures induced higher antibody titers than do non-O-acetylated L3,9 structures (43). Polyclonal antiserum raised against L2 and L5 immunotypes was bactericidal, but immunotype specific, whilst the response elicited by L3,9 immunotypes recognized common epitopes preserved between L2 and L3,9 (44) but was not bactericidal.

Present strategies for the development of LOS glycoconjugates as meningococcal vaccine candidates are providing evidence that such vaccines yield cross protective immunity against meningococci (45). Because the majority of N. meningitidis isolated from invasive disease express the L[3,7,9] immunotype LOS (3), (4) and because the expression of the L[3,7,9] immunotype LOS by N. meningitidis increases resistance to complement-mediated lysis during disseminated disease (19), most of this work has focused upon utilizing an L3 LOS. However, studies assessing the distribution of LOS immunotypes expressed in meningococcal disease isolates indicate that at least one-quarter of strains causing invasive meningococcal disease express L[2,4], and these strains may utilize other mechanisms for resistance to complement-mediated lysis (46). Based upon our current and previous observations of strain NMB (29), the steps involved for conversion among immunotypes include the phase variable expression of IgtA, the carriage of an active lot3, the acquisition of islets carrying Ipt-6 and IgtG, and the phase variable and regulated expression of IgtG. The mechanisms of LOS assembly and the basis of immunotype switching are important in both understanding meningococcal pathogenesis and for LOS vaccine design. O-acetylation of meningococcal LOS represents a potential mechanism for immune escape from a vaccine targeting only L3 immunotype LOS, and therefore multiple endotoxin structures are believed to be required for an effective meningococcal endotoxin-based vaccine.

Expression refers to the transcription and translation of a structural gene (coding sequence) so that a protein (i.e., expression product) having the biological activity of the O-acetyltransferase as described herein is synthesized. It is understood that post-translational modification(s) in certain types of recombinant host cells may remove portions of the polypeptide which are not essential to enzymatic activity.

The term expression control sequences refer to DNA sequences that control and regulate the transcription and translation of another DNA sequence (i.e., a coding sequence). A coding sequence is operatively linked to an expression control sequence when the expression control sequence controls and regulates the transcription and translation of that coding sequence. Expression control sequences include, but are not limited to, promoters, enhancers, promoter-associated regulatory sequences, transcription termination and polyadenylation sequences, and their positioning and use is well understood by the ordinary skilled artisan.

The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene. The combination of the expression control sequences and the Lot3 O-acetyltransferase coding sequence form the Lot3 O-acetyltransferase expression cassette.

As used herein, an exogenous or heterologous nucleotide sequence is one which is not in nature covalently linked to a particular nucleotide sequence, e.g., a Lot3 O-acetyltransferase coding sequence. Examples of exogenous nucleotide sequences include, but are not limited to, plasmid vector sequences, expression control sequences not naturally associated with particular Lot3 O-acetyltransferase coding sequences, and viral or other vector sequences. A non-naturally occurring DNA molecule is one which does not occur in nature, and it is thus distinguished from a chromosome, or example. As used herein, a non-naturally occurring DNA molecule comprising a sequence encoding an expression product with Lot3 O-acetyltransferase activity is one which comprises said coding sequence and sequences which are not associated therewith in nature.

Similarly, as used herein an exogenous gene is one which does not naturally occur in a particular recombinant host cell but has been introduced in using genetic engineering techniques well known in the art. An exogenous gene as used herein can comprise a Lot3 O-acetyltransferase coding sequence expressed under the control of an expression control sequence not associated in nature with said coding sequence.

Another feature is the expression of the sequences encoding Lot3 O-acetyltransferase. As is well-known in the art, DNA sequences may be expressed by operatively linking them to an expression control sequence in an appropriate expression vector and employing that expression vector to transform an appropriate host cell.

A wide variety of host/expression vector combinations may be employed in expressing the DNA sequences taught herein. Useful expression vectors, for example, may consist of segments of chromosomal, nonchromosomal and synthetic DNA sequences. Suitable vectors include derivatives of SV40 and known bacterial plasmids, e.g., Escherichia coli plasmids colE1, pCR1, pBR322, pMB9 and their derivatives, plasmids such as RP4; phage DNAs, e.g., M13 derivatives, the numerous derivatives of phage λ, e.g., λgt11, and other phage DNA; yeast plasmids derived from the 2μ circle; vectors useful in eukaryotic cells, such as insect or mammalian cells; vectors derived from combinations of plasmids and phage DNAs, such as plasmids that have been modified to employ phage DNA or other expression control sequences; baculovirus derivatives; and the like. For mammalian cells there are a number of well-known expression vectors available to the art.

Any of a wide variety of expression control sequences may be used in these vectors to express the DNA sequences taught herein. Such useful expression control sequences include, for example, the early and late promoters of SV40 or adenovirus for expression in mammalian cells, the lac system, the trp system, the tac or trc system, the major operator and promoter regions of phage A, the control regions of fd coat protein, the promoter for 3-phosphoglycerate kinase of phosphatase (e.g., pho5), the promoters of the yeast α-mating factors, and other sequences known to control the expression of genes of prokaryotic or eukaryotic cells or their viruses, and various combinations thereof. The skilled artisan understands which expression control sequences are appropriate to particular vectors and host cells.

A wide variety of host cells are also useful in expressing the DNA sequences taught herein. These hosts may include well-known prokaryotic and eukaryotic hosts, such as strains of E. coli, Pseudomonas, Bacillus, Streptomyces, fungi such as yeasts, and animal cells, such as Chinese Hamster Ovary (CHO), R1.1, B-W and L-M cells, African Green Monkey kidney cells (e.g., COS 1, COS-7, BSC1, BSC40, and BMT10), insect cells (e.g., Sf9), and human cells and plant cells in culture.

It is understood that not all combinations of vector, expression control sequence and host cell will function equally well to express the DNA sequences encoding the acetyl-transferase as taught herein. However, one skilled in the art can select the proper vector, expression control sequence, and host cell combination without undue experimentation to accomplish the desired expression.

In selecting a suitable expression control sequence, a variety of factors are normally considered. These include, for example, the relative strength of the promoter, its controllability, and its compatibility with the particular DNA sequence or gene to be expressed, e.g., with regard to potential secondary structure. Suitable hosts are selected by consideration of factors including compatibility with the chosen vector, secretion characteristics, ability to fold proteins correctly, and fermentation requirements, as well as any toxicity to the host of the product encoded by the DNA sequences to be expressed, and the ease of purification of the expression products. The practitioner can select the appropriate host cells and expression mechanisms for a particular purpose.

Several strategies are available for the isolation and purification of recombinant Lot3 O-acetyltransferase after expression in a host system. One method involves expressing the proteins in bacterial cells, lysing the cells, and purifying the protein by conventional means. Alternatively, one can engineer the DNA sequences for secretion from cells. A Lot3 O-acetyltransferase protein can be readily engineered to facilitate purification and/or immobilization to a solid support of choice. For example, a stretch of 6-8 histidine residues can be engineered through polymerase chain reaction or other recombinant DNA technology to allow purification of expressed recombinant protein over a nickel-charged nitrilotriacetic acid (NTA) column using commercially available materials. Other oligopeptide “tags” which can be fused to a protein of interest by such techniques include, without limitation, strep-tag (Sigma-Genosys, The Woodlands, Tex.) which directs binding to streptavidin or its derivative streptactin (Sigma-Genosys); a glutathione-A-transferase gene fusion system which directs binding to glutathione coupled to a solid support (Amersham Pharmacia Biotech, Uppsala, Sweden); a calmodulin-binding peptide fusion system which allows purification using a calmodulin resin (Stratagene, La Jolla, Calif.); a maltose binding protein fusion system allowing binding to an amylose resin (New England Biolabs, Beverly, Mass.); and an oligo-histidine fusion peptide system which allows purification using a Ni²⁺-NTA column (Qiagen, Valencia, Calif.).

Coding sequences which are synonymous to the coding sequence provided herein are within the scope of the present invention, as are sequences encoding O-acetyltransferases carrying out the same acetylations of Neisseria meningitidis lipooligosaccharides or oligosaccharides, and where those sequences encode Lot3 O-acetyltransferases with at least 80% amino acid sequence identity with that of either the Z2491 or the NMB enzyme. All integers between 80 and 100% are included within the scope of the present invention in this context. In calculations of amino acid sequence identify, gaps inserted to optimize alignment are treated as mismatches.

Lot3 O-acetyltransferase coding sequences from various N. meningitidis strains have significant sequence homology to the specifically exemplified O-acetyltransferase coding sequences, and the encoded enzymes have a high degree of amino acid sequence identity as disclosed herein. It is obvious to one normally skilled in the art that equivalent clones and PCR amplification products can be readily isolated using standard procedures and the sequence information provided herein. The ordinary skilled artisan can utilize the exemplified sequences provided herein, or portions thereof, preferably at least 25-30 bases in length, in hybridization probes to identify cDNA (or genomic) clones encoding Lot3 O-acetyltransferase, where there is at least 70% sequence homology to the probe sequence using appropriate art-known hybridization techniques. The skilled artisan understands that the capacity of a cloned cDNA to encode functional Lot3 O-acetyltransferase enzyme can be readily tested as taught herein.

Hybridization conditions appropriate for detecting various extents of nucleotide sequence homology between probe and target sequences and theoretical and practical consideration are given, for example in B. D. Hames and S. J. Higgins (1985) Nucleic Acid Hybridization, IRL Press, Oxford, and in Sambrook et al. (1989) supra. Under particular hybridization conditions the DNA sequences taught herein hybridize to other DNA sequences having sufficient homology, including homologous sequences from different species. It is understood in the art that the stringency of hybridization conditions is a factor in the degree of homology required for hybridization. The skilled artisan knows how to manipulate the hybridization conditions so that the stringency of hybridization is at the desired level (high, medium). If attempts to identify and isolate the Lot3 O-acetyltransferase coding sequence from another N. meningitidis strain fail using high stringency conditions, the skilled artisan understands how to decrease the stringency of the hybridization conditions so that a sequence with a lower degree of sequence homology hybridizes to the sequence used as a probe. The choice of the length and sequence of the probe is readily understood by the skilled artisan.

The DNA sequences encoding a Lot3 O-acetyl transferase can be prepared or isolated using recombinant DNA techniques. These include cDNA sequences, sequences isolated using PCR, DNA sequences isolated from their native genome, and synthetic DNA sequences. As used herein, this term is not intended to encompass naturally-occurring chromosomes or genomes. These sequences can be used to direct recombinant synthesis of Lot3 O-acetyltransferase for enzymatic acetylation of isolated OS and/or LOS, especially from N. meningitidis strains.

Isolated oligosaccharides and/or lipooligosaccharides are separated from the cells and culture medium from which it was produced. Further purification is optional and within the realm of the skilled artisan.

In the present context, an in vitro enzymatic reaction, especially acetylation of N. meningitidis oligosaccharide and lipooligosaccharide is carried out in the absence of whole, live cells. The enzyme source can be a purified or partly purified enzyme or it can be present in a cell extract, recombinantly produced or otherwise, although greater amounts per cell are produced through recombinant DNA technology. Highly acetylated OS/LOS preparations can be isolated from N. meningitidis strains that contain and express an additional lot3 gene or which have been genetically modified to contain and express a lot3 coding sequence at levels greater than is wild type N. meningitidis cells.

It is well-known in the biological arts that certain amino acid substitutions can be made within a protein without affecting the functioning of that protein. Preferably such substitutions are of amino acids similar in size and/or charge properties. For example, Dayhoff et al. (1978) in Atlas of Protein Sequence and Structure, Volume 5, Supplement 3, Chapter 22, pages 345-352, which is incorporated by reference herein, provides frequency tables for amino acid substitutions which can be employed as a measure of amino acid similarity. Dayhoff et al.'s frequency tables are based on comparisons of amino acid sequences for proteins having the same function from a variety of evolutionarily different sources.

It will be a matter of routine experimentation for the ordinary skilled artisan to use the DNA sequence information presented herein to optimize Lot3 O-acetyltransferase expression in a particular expression vector and cell line for a desired purpose. A cell line genetically engineered to contain and express a Lot3 O-acetyltransferase coding sequence is useful for the recombinant expression of protein products with the characteristic enzymatic activity of the specifically exemplified enzyme. Any means known to the art can be used to introduce an expressible Lot3 O-acetyltransferase coding sequence into a cell to produce a recombinant host cell, i.e., to genetically engineer such a recombinant host cell. Recombinant host cell lines which express high levels of Lot3 O-acetyltransferase are useful as sources for the purification of this enzyme, especially for in vitro acetylation of isolated OS or LOS polysaccharides, desirably those from N. meningitidis.

The amino acids which occur in the various amino acid sequences referred to in the specification have their usual three- and one-letter abbreviations routinely used in the art: A, Ala, Alanine; C, Cys, Cysteine; D, Asp, Aspartic Acid; E, Glu, Glutamic Acid; F, Phe, Phenylalanine; G, Gly, Glycine; H, His, Histidine; I, Iie, Isoleucine; K, Lys, Lysine; L, Leu, Leucine; M, Met, Methionine; N, Asn, Asparagine; P, Pro, Proline; Q, Gln, Glutamine; R, Arg, Arginine; S, Ser, Serine; T, Thr, Threonine; V, Val, Valine; W, Try, Tryptophan; Y, Tyr, Tyrosine.

A protein is considered an isolated protein if it is a protein isolated from a host cell in which it is recombinantly produced. It can be purified or it can simply be free of other proteins and biological materials with which it is associated in nature.

An isolated nucleic acid is a nucleic acid the structure of which is not identical to that of any naturally occurring nucleic acid or to that of any fragment of a naturally occurring genomic nucleic acid spanning more than three separate genes. The term therefore covers, for example, a DNA which has the sequence of part of a naturally occurring genomic DNA molecule but is not flanked by both of the coding or noncoding sequences that flank that part of the molecule in the genome of the organism in which it naturally occurs; a nucleic acid incorporated into a vector or into the genomic DNA of a prokaryote or eukaryote in a manner such that the resulting molecule is not identical to any naturally occurring vector or genomic DNA; a separate molecule such as a cDNA, a genomic fragment, a fragment produced by polymerase chain reaction (PCR), or a restriction fragment; and a recombinant nucleotide sequence that is part of a hybrid gene, i.e., a gene encoding a fusion protein. Specifically excluded from this definition are nucleic acids present in mixtures of DNA molecules, transformed or transfected cells, and cell clones, e.g., as these occur in a DNA library such as a cDNA or genomic DNA library.

In the present context, a promoter is a DNA region which includes sequences sufficient to cause transcription of an associated (downstream) sequence. The promoter may be regulated, i.e., not constitutively acting to cause transcription of the associated sequence. If inducible, there are sequences present which mediate regulation of expression so that the associated sequence is transcribed only when an inducer molecule is present in the medium in or on which the organism is cultivated.

One DNA portion or sequence is downstream of second DNA portion or sequence when it is located 3′ of the second sequence. One DNA portion or sequence is upstream of a second DNA portion or sequence when it is located 5′ of that sequence.

One DNA molecule or sequence and another are heterologous to another if the two are not derived from the same ultimate natural source. The sequences may be natural sequences, or at least one sequence can be designed by man, as in the case of a multiple cloning site region. The two sequences can be derived from two different species or one sequence can be produced by chemical synthesis provided that the nucleotide sequence of the synthesized portion was not derived from the same organism as the other sequence.

An isolated or substantially pure nucleic acid molecule or polynucleotide is an O-acetyltransferase-encoding polynucleotide which is substantially separated from other polynucleotide sequences which naturally accompany it on the bacterial chromosome. The term embraces a polynucleotide sequence which has been removed from its naturally occurring environment, and includes recombinant or cloned DNA isolates, chemically synthesized analogues and analogues biologically synthesized by heterologous systems.

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

A nucleotide sequence is operably linked when it is placed into a functional relationship with another nucleotide sequence. For instance, a promoter is operably linked to a coding sequence if the promoter effects its transcription or expression. Generally, operably linked means that the sequences being linked are contiguous and, where necessary to join two protein coding regions, contiguous and in reading frame. However, it is well known that certain genetic elements, such as enhancers, may be operably linked even at a distance, i.e., even if not contiguous.

The term recombinant polynucleotide refers to a polynucleotide which is made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

Polynucleotide probes include an isolated polynucleotide attached to a label or reporter molecule and may be used to identify and isolate other O-acetyltransferase coding sequences equivalent in function to the lot3 specifically exemplified herein, for example, those from others strains of N. meningitidis. Probes comprising synthetic oligonucleotides or other polynucleotides may be derived from naturally occurring or recombinant single or double stranded nucleic acids or be chemically synthesized. Polynucleotide probes may be labeled by any of the methods known in the art, e.g., random hexamer labeling, nick translation, or the Klenow fill-in reaction, or with fluors or other detectable moieties.

Large amounts of the polynucleotides may be produced by replication in a suitable host cell. Natural or synthetic DNA fragments coding for a protein of interest are incorporated into recombinant polynucleotide constructs, typically DNA constructs, capable of introduction into and replication in a prokaryotic or eukaryotic cell, especially cultured mammalian cells, wherein protein expression is desired. Usually the construct is suitable for replication in a host cell, such as cultured mammalian cell or a bacterium, but a multicellular eukaryotic host may also be appropriate, with or without integration within the genome of the host cell. Commonly used prokaryotic hosts include strains of Escherichia coli, although other prokaryotes, such as Bacillus subtilis or a pseudomonad, may also be used. Eukaryotic host cells include mammalian cells, yeast, filamentous fungi, plant, insect, amphibian and avian cell lines. Such factors as ease of manipulation, ability to appropriately glycosylate expressed proteins, degree and control of recombinant protein expression, ease of purification of expressed proteins away from cellular contaminants or other factors influence the choice of the host cell.

The polynucleotides may also be produced by chemical synthesis, e.g., by the phosphoramidite method described by Beaucage and Caruthers (1981) Tetra. Letts. 22: 1859-1862 or the triester method according to Matteuci et al. (1981) J. Am. Chem. Soc. 103: 3185, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence. DNA constructs prepared for introduction into a prokaryotic or eukaryotic host will typically comprise a replication system (i.e. vector) recognized by the host, including the intended DNA fragment encoding the desired polypeptide, and will preferably also include transcription and translational initiation regulatory sequences operably linked to the polypeptide-encoding segment. Expression systems (expression vectors) may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides may also be included where appropriate from secreted polypeptides of the same or related species, which allow the protein to cross and/or lodge in cell membranes or be secreted from the cell.

An appropriate promoter and other necessary vector sequences will be selected so as to be functional in the host. Examples of workable combinations of cell lines and expression vectors are described in Sambrook et al. (1989) vide infra; Ausubel et al. (Eds.) (1995) Current Protocols in Molecular Biology, Greene Publishing and Wiley Interscience, New York; and Metzger et al. (1988) Nature, 334: 31-36. Many useful vectors for expression in bacteria, yeast, fungal, mammalian, insect, plant or other cells are well known in the art and may be obtained from such vendors as Stratagene, New England Biolabs, Promega Biotech, and others. In addition, the construct may be joined to an amplifiable gene (e.g., DHFR) so that multiple copies of the gene may be made. For appropriate enhancer and other expression control sequences, see also Enhancers and Eukaryotic Gene Expression, Cold Spring Harbor Press, N.Y. (1983). While such expression vectors may replicate autonomously, they may less preferably replicate by being inserted into the genome of the host cell.

Expression and cloning vectors will likely contain a selectable marker, that is, a gene encoding a protein necessary for the survival or growth of a host cell transformed with the vector. Although such a marker gene may be carried on another polynucleotide sequence co-introduced into the host cell, it is most often contained on the cloning vector. Only those host cells into which the marker gene has been introduced will survive and/or grow under selective conditions. Typical selection genes encode proteins that confer resistance to antibiotics or other toxic substances, e.g., ampicillin, neomycin, methotrexate, etc.; complement auxotrophic deficiencies; or supply critical nutrients not available from complex media. The choice of the proper selectable marker will depend on the host cell; appropriate markers for different hosts are known in the art.

Recombinant host cells, in the present context, are those which have been genetically modified to contain an isolated DNA molecule encoding a Lot3 O-acetyltransferase The DNA can be introduced by any means known to the art which is appropriate for the particular type of cell, including without limitation, transfection, transformation, lipofection or electroporation.

It is recognized by those skilled in the art that the DNA sequences may vary due to the degeneracy of the genetic code and codon usage. All (synonymous) DNA sequences which code for the O-acetyltransferase protein are included within the scope of this invention, including the DNA sequence as given in Table 6A. Also contemplated are coding sequences which encode an O-acetyltransferase as taught herein with at least 80%, at least 85%, at least 90%, at least 95% or at least 98% amino acid sequence identity to that of Table 5, SEQ ID NO:5. A review of Table 5 reveals that, at a minimum, amino acids 3, 22, 28, 168, 169, 181, 187, 238, 291, 446, 447, 497, 560, 583, 611, 615, 617, 618 and 619 can vary from SEQ ID NO:5. Members of Table 1 have from 98.6 to greater than 99% sequence identity with SEQ ID NO:5 (amino acids matching SEQ ID NO:5 divided by 622 amino acids time 100%). For an amino acid sequence into which a gap(s) must be introduced to improve alignment, the gap(s) is treated as a mismatched amino acid. Similarly, if an amino acid sequence requires the deletion of one or more amino acids to improve alignment with SEQ ID NO:5, those deleted amino acids are treated as a mismatch. It is understood that there can be up to 5 additional substitution, insertion or deletion mutations in any of SEQ ID NO:5 without loss of enzymatic activity; the skilled artisan can readily test any variant to confirm that the enzyme activity is present.

Additionally, it is recognized by those skilled in the art that allelic variations may occur in the DNA sequences which do not significantly change activity of the amino acid sequences of the peptides which the DNA sequences encode. All such equivalent DNA sequences are included within the scope of this invention and the definition of the regulated promoter region. The skilled artisan understands that the sequence of the exemplified O-acetyltransferase protein and the nucleotide sequence encoding it can be used to identify and isolate additional, nonexemplified nucleotide sequences which are functionally equivalent to the sequences given FIG. 8A.

Hybridization procedures are useful for identifying polynucleotides with sufficient homology to the subject coding sequence to be useful as taught herein. The particular hybridization technique is not essential to the subject invention. As improvements are made in hybridization techniques, they can be readily applied by one of ordinary skill in the art.

A probe and sample are combined in a hybridization buffer solution and held at an appropriate temperature until annealing occurs. Thereafter, the membrane is washed free of extraneous materials, leaving the sample and bound probe molecules typically detected and quantified by autoradiography and/or liquid scintillation counting. As is well known in the art, if the probe molecule and nucleic acid sample hybridize by forming a strong non-covalent bond between the two molecules, it can be reasonably assumed that the probe and sample are essentially identical, or completely complementary, if the annealing and washing steps are carried out under conditions of high stringency. The probe's detectable label provides a means for determining whether hybridization has occurred.

In the use of the oligonucleotides or polynucleotides as probes, the particular probe is labeled with any suitable label known to those skilled in the art, including radioactive and non-radioactive labels. Typical radioactive labels include ³²P, ³⁵S, or the like. Non-radioactive labels include, for example, ligands such as biotin or thyroxine, as well as enzymes such as hydrolases or peroxidases, or a chemiluminescent reagent such as luciferin, or fluorescent compounds like fluorescein and its derivatives. Alternatively, the probes can be made inherently fluorescent as described in International Application No. WO 93/16094.

Various degrees of stringency of hybridization can be employed. The more stringent the conditions, the greater the complementarity that is required for duplex formation. Stringency can be controlled by temperature, probe concentration, probe length, ionic strength, time, and the like. Preferably, hybridization is conducted under moderate to high stringency conditions by techniques well know in the art, as described, for example in Keller, G. H., M. M. Manak (1987) DNA Probes, Stockton Press, New York, N.Y., pp. 169-170, hereby incorporated by reference.

As used herein, moderate to high stringency conditions for hybridization are conditions which are particularly advantageous. An example of high stringency conditions are hybridizing at 68° C. in 5×SSC/5×Denhardt=s solution/0.1% SDS, and washing in 0.2×SSC/0.1% SDS at room temperature. An example of conditions of moderate stringency are hybridizing at 68° C. in 5×SSC/5×Denhardt=s solution/0.1% SDS and washing at 42° C. in 3×SSC. The parameters of temperature and salt concentration can be varied to achieve the desired level of sequence identity between probe and target nucleic acid. See, e.g., Sambrook et al. (1989) vide infra or Ausubel et al. (1995) Current Protocols in Molecular Biology, John Wiley & Sons, NY, N.Y., for further guidance on hybridization conditions.

Specifically, hybridization of immobilized DNA in Southern blots with ³²P-labeled gene specific probes is performed according to standard methods (Maniatis et al.) In general, hybridization and subsequent washes were carried out under moderate to high stringency conditions that allowed for detection of target sequences with homology to the exemplified sequence. For double-stranded DNA gene probes, hybridization can be carried out overnight at 20-25° C. below the melting temperature (Tm) of the DNA hybrid in 6×SSPE 5×Denhardt=s solution, 0.1% SDS, 0.1 mg/ml denatured DNA. The melting temperature is described by the following formula (Beltz et al. (1983) Methods of Enzymology, R. Wu, L, Grossman and K Moldave (eds) Academic Press, New York 100:266-285).

Tm=81.5° C.+16.6 Log [Na+]+0.41 (+G+C)−0.61(% formamide)−600/length of duplex in base pairs.

Washes are typically carried out as follows: twice at room temperature for 15 minutes in 1×SSPE, 0.1% SDS (low stringency wash), and once at Tm-20° C. for 15 minutes in 0.2×SSPE, 0.1% SDS (moderate stringency wash).

For oligonucleotide probes, hybridization is carried out overnight at 10-20° C. below the melting temperature (Tm) of the hybrid 6×SSPE, 5×Denhardt=s solution, 0.1% SDS, 0.1 mg/ml denatured DNA. Tm for oligonucleotide probes is determined by the following formula: TM(° C.)=2(number T/A base pairs +4(number G/C base pairs) (Suggs et al. (1981) ICB-UCLA Symp. Dev. Biol. Using Purified Genes, D. D. Brown (ed.), Academic Press, New York, 23:683-693).

Washes are typically carried out as follows: twice at room temperature for 15 minutes 1×SSPE, 0.1% SDS (low stringency wash), and once at the hybridization temperature for 15 minutes in 1×SSPE, 0.1% SDS (moderate stringency wash).

In general, salt and/or temperature can be altered to change stringency. With a labeled DNA fragment >70 or so bases in length, the following conditions can be used: Low, 1 or 2×SSPE, room temperature; Low, 1 or 2×SSPE, 42° C.; Moderate, 0.2× or 1×SSPE, 65° C.; and High, 0.1×SSPE, 65° C.

Duplex formation and stability depend on substantial complementarity between the two strands of a hybrid, and, as noted above, a certain degree of mismatch can be tolerated. Therefore, the probe sequences useful for binding to a Lot3 O-acetyltransferase coding sequence include mutations (both single and multiple), deletions, insertions of the described sequences, and combinations thereof, wherein said mutations, insertions and deletions permit formation of stable hybrids with the target polynucleotide of interest. Mutations, insertions, and deletions can be produced in a given polynucleotide sequence in many ways, and those methods are known to an ordinarily skilled artisan. Other methods may become known in the future.

Thus, mutational, insertional, and deletional variants of the disclosed nucleotide sequences can be readily prepared by methods which are well known to those skilled in the art. These variants can be used in the same manner as the exemplified primer sequences so long as the variants have substantial sequence identity with the original sequence. As used herein, substantial sequence identity refers to identity which is sufficient to enable the variant polynucleotide to function in the same capacity as the polynucleotide from which the probe was derived. Preferably, this identity is greater than 80%, more preferably, this identity is greater than 85%, even more preferably this identity is greater than 90%, and most preferably, this identity is greater than 95%. All integers between 80 and 100%. The degree of (homology or) identity needed for the variant to function in its intended capacity depends upon the intended use of the sequence. It is well within the skill of a person trained in this art to make mutational, insertional, and deletional mutations which are equivalent in function or are designed to improve the function of the sequence or otherwise provide a methodological advantage.

Polymerase Chain Reaction (PCR) is a repetitive, enzymatic, primed amplification of a nucleic acid sequence. This procedure is well known and commonly used by those skilled in this art (see, e.g., Mullis, U.S. Pat. Nos. 4,683,195, 4,683,202, and 4,800,159; Saiki et al. (1985) Science 230:1350-1354). The thermostable Taq polymerase, which is isolated from the thermophilic bacterium Thermus aquaticus, allows automation of the amplification process. Other enzymes which can be used are known to those skilled in the art.

It is well known in the art that the polynucleotide sequences encoding a Lot3 O-acetyltransferase can be truncated and/or mutated such that certain of the resulting fragments and/or mutants of the original full-length sequence can retain the desired characteristics of the full-length sequence. A wide variety of restriction enzymes which are suitable for generating fragments from larger nucleic acid molecules are well known. In addition, it is well known that Bal31 exonuclease can be conveniently used for time-controlled limited digestion of DNA. See, for example, Maniatis (1982) Molecular Cloning: A Laboratory Manual, Cold Spring Harbor Laboratory, New York, pages 135-139, incorporated herein by reference. See also Wei et al. (1983 J. Biol. Chem. 258:13006-13512. By use of Bal31 exonuclease (commonly referred to as Aerase-a-base@ procedures), the ordinarily skilled artisan can remove nucleotides from either or both ends of the subject nucleic acids to generate a wide spectrum of fragments which are functionally equivalent to the subject nucleotide sequences. One of ordinary skill in the art can, in this manner, generate hundreds of fragments of controlled, varying lengths from locations all along the original O-acetyltransferase coding sequence. The ordinarily skilled artisan can routinely test or screen the generated fragments for their characteristics and determine the utility of the fragments as taught herein. It is also well known that the mutant sequences of the full length sequence, or fragments thereof, can be easily produced with site directed mutagenesis. See, for example, Larionov, O. A. and Nikiforov, V. G. (1982) Genetika 18(3):349-59; Shortle, D, DiMaio, D., and Nathans, D. (1981) Annu. Rev. Genet. 15:265-94; both incorporated herein by reference. The skilled artisan can routinely produce deletion-, insertion-, or substitution-type mutations and identify those resulting mutants which contain the desired characteristics of the full length wild-type sequence, or fragments thereof, i.e., those which retain O-acetyltransferase activity as determined herein.

DNA sequences having at least 80, 90, or at least 95% (and all integers and ranges between 80 and 100%) identity to the recited Lot3 coding sequences disclosed herein and functioning to encode a Lot3 O-acetyltransferase protein are within the scope of this invention. Such functional equivalents are included in the definition of an O-acetyltransferase coding sequence. Additional Lot coding sequences from other immunotypes and from other neisserial species are provided herein below. Following the teachings herein and using knowledge and techniques well known in the art, the skilled worker will be able to make a large number of operative embodiments having equivalent DNA sequences to those listed herein without the expense of undue experimentation.

As used herein percent sequence identity of two nucleic acids is determined using the algorithm of Altschul et al. (1997) Nucl. Acids Res. 25: 3389-3402; see also Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See the National Center for Biotechnology Information website and programs available on the internet and from other sources.

In another embodiment, immunogenic compositions for producing polyclonal and/or monoclonal antibodies capable of specifically binding to O-acetyltransferase, OS or LOS from N. meningitidis (or fragments thereof) are provided. Similar material can be prepared from other Lot-expressing neisserial strains in for use in immunogenic compositions as well. The term antibody is used to refer both to a homogenous molecular entity and a mixture such as a serum product made up of a plurality of different molecular entities. Monoclonal or polyclonal antibodies, preferably monoclonal, specifically reacting with a particular epitope in a molecule of interest may be made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1993) supra. Also, recombinant immunoglobulins may be produced by any methods known in the art, including but not limited to, the methods described in U.S. Pat. No. 4,816,567, incorporated by reference herein. Monoclonal antibodies with affinities of 10⁸ M⁻¹, preferably 10⁹ to 10¹⁰ or more are preferred.

Antibodies generated against a molecule of interest are useful, for example, as probes for screening DNA expression libraries or for detecting the presence of particular neisserial strains or their isolated LOS or OS polysaccharides in a test sample. Hydrophilic regions of the Lot3 O-acetyltransferase as taught herein can be identified by the skilled artisan, and peptide antigens can be synthesized and conjugated to a suitable carrier protein (e.g., bovine serum albumin or keyhole limpet hemocyanin) for use in vaccines or in raising antibody specific for LOS biosynthetic proteins. Frequently, the polypeptides and antibodies will be labeled by joining, either covalently or noncovalently, a substance which provides a detectable signal. Suitable labels include but are not limited to radionuclides, enzymes, substrates, cofactors, inhibitors, fluorescent agents, chemiluminescent agents, magnetic particles and the like. United States patents describing the use of such labels include but are not limited to U.S. Pat. Nos. 3,817,837; 3,850,752; 3,939,350; 3,996,345; 4,277,437; 4,275,149; and 4,366,241.

Antibodies specific for the acetylated LOS from N. meningitidis are useful in preventing disease resulting from neisseriae, especially N. meningitidis infections. Such antibodies can be obtained by the methods described above. Because there is some loss of acetyl residues during isolation of the OS and/or LOS and because there is some loss of immunogenicity of an unacetylated or a poorly acetylated OS or LOS, the quality of a N. meningitidis OS and/or LOS-containing immunogenic composition, it is advantageous to treat such a preparation with the Lot3 O-acetyltransferase as taught herein prior to use in immunogenic compositions, including vaccine compositions. Techniques applied to in vitro acetylation of capsular polysaccharide can be applied to isolated OS and/or LOS; see, e.g., US Patent Publication 2006/0073168 A1.

Compositions and immunogenic preparations, including vaccine compositions comprising in vitro acetylated oligosaccharides and lipooligosaccharides from N. meningitidis or nonacetylated preparations, and a suitable carrier therefor are provided. Immunogenic compositions can be similarly prepared using material from other Neisserial species as well. Immunogenic compositions are those which result in specific antibody production when injected into a human or an animal. Such immunogenic compositions are useful, for example, in immunizing a human, against infection by neisserial pathogenic strains, especially those of N. meningitidis. The immunogenic preparations comprise an immunogenic amount of an acetylated LOS (or OS) preparation derived from a N. meningitidis strain and a suitable carrier.

The immunogenic compositions advantageously further comprise lipooligosaccharide(s), proteins and/or neisserial cells of N. meningitidis and optionally, one or more other serogroups and/or immunotypes, including, but not limited to, any known to the art. It is understand that where whole cells are formulated into the immunogenic composition, the cells are preferably inactivated, especially if the cells are of a virulent strain. Such immunogenic compositions may comprise one or more LOS preparations, capsular polysaccharides and/or another protein or other immunogenic cellular component. By “immunogenic amount” is meant an amount capable of eliciting the production of antibodies directed against neisserial (acetylated or nonacetylated, as desired) OS or LOS polysaccharides in an animal or human to which the vaccine or immunogenic composition has been administered.

Immunogenic carriers may be used to enhance the immunogenicity of a component of the immunogenic composition as known to the art. Such carriers include, but are not limited to, proteins and polysaccharides, liposomes, and bacterial cells and membranes. Protein carriers may be joined to the molecule(s) of interest to form fusion proteins by recombinant or synthetic means or by chemical coupling. Useful carriers and means of coupling such carriers to polypeptide antigens are known in the art. The art knows how to administer immunogenic compositions so as to generate protective immunity on the mucosal surfaces of the upper respiratory system, especially the mucosal epithelium of the nasopharynx, where immunity is specific for N. meningitidis, as well as protecting other parts of the body.

The immunogenic compositions provided herein may be formulated by any means known in the art. Such vaccines are typically prepared as injectables, either as liquid solutions or suspensions. Solid forms suitable for solution in, or suspension in, liquid prior to injection may also be prepared. The preparation may also, for example, be emulsified, or the protein encapsulated in liposomes.

The active immunogenic ingredients are often mixed with excipients or carriers which are pharmaceutically acceptable and compatible with the active ingredient. Suitable excipients include but are not limited to water, saline, dextrose, glycerol, ethanol, or the like and combinations thereof. The concentration of the immunogenic polypeptide in injectable formulations is usually in the range of 0.2 to 5 mg/ml.

In addition, if desired, the vaccines may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A referred to as MTP-PE); and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against the immunogen resulting from administration of the immunogen in vaccines which are also comprised of the various adjuvants. Such additional formulations and modes of administration as are known in the art may also be used.

In vitro acetylated lipooligosaccharide or oligosaccharides from N. meningitidis and advantageously containing cells of N. meningitidis may be formulated into immunogenic compositions as neutral or salt forms. Preferably, when whole cells are used, they are of attenuated or avirulent strains, or the cells are killed before use. Pharmaceutically acceptable salts include but are not limited to the acid addition salts (formed with free amino groups of the peptide) which are formed with inorganic acids, e.g., hydrochloric acid or phosphoric acids; and organic acids, e.g., acetic, oxalic, tartaric, or maleic acid. Salts formed with the free carboxyl groups may also be derived from inorganic bases, e.g., sodium, potassium, ammonium, calcium, or ferric hydroxides, and organic bases, e.g., isopropylamine, trimethylamine, 2-ethylamino-ethanol, histidine, and procaine.

The immunogenic preparations provided herein are administered in a manner compatible with the dosage formulation, and in such amount as will be prophylactically and/or therapeutically effective. The quantity to be administered, which is generally in the range of about 100 to 1,000 μg of in vitro acetylated oligosaccharide and/or lipooligosaccharide per dose, more generally in the range of about 1 to 500 μg per dose, depends on the subject to be treated, the capacity of the individual's immune system to synthesize antibodies, and the degree of protection desired. Precise amounts of the active ingredient required to be administered may depend on the judgment of the physician and may be peculiar to each individual, but such a determination is within the skill of such a practitioner.

The vaccine or other immunogenic composition may be given in a single dose or multiple dose schedule. A multiple dose schedule is one in which a primary course of vaccination may include 1 to 10 or more separate doses, followed by other doses administered at subsequent time intervals as required to maintain and or reinforce the immune response, e.g., at 1 to 4 months for a second dose, and if needed, a subsequent dose(s) after several months.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the functional and novel characteristics of the claim. Any recitation herein of the term “comprising”, particularly in a description of components of a composition or in a description of elements of a device, is understood to encompass those compositions and methods consisting essentially of and consisting of the recited components or elements. The invention may be practiced in the absence of any element or elements, limitation or limitations which is not specifically disclosed herein, provided that there would be no anticipation by or obviousness over prior art.

The terms and expressions which have been employed are used as terms of description and not of limitation, and there is no intention that in the use of such terms and expressions of excluding any equivalents of the features shown and described or portions thereof, but it is recognized that various modifications are possible without going beyond the scope of the invention claimed. Thus, it should be understood that although embodiments of the present invention have been specifically disclosed by certain embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art.

All patents and publications mentioned in the specification are indicative of the levels of skill of those skilled in the art(s) to which the invention pertains, and all references cited herein are hereby incorporated by reference to the extent that there is no inconsistency with the present specification.

Monoclonal or polyclonal antibodies, preferably monoclonal, specifically reacting with a polypeptide or protein of interest may be made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1995) Current Protocols in Molecular Biology, Wiley Interscience, New York, N.Y.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering Principles and Methods, Vols. 1-4, Plenum Press, New York; and Ausubel et al. (1995) Current Protocols in Molecular Biology, Greene/Wiley, New York, N.Y. Abbreviations and nomenclature, where employed, are deemed standard in the field and commonly used in professional journals such as those cited herein.

The specifically exemplified sequences, compositions, cells, vectors, compounds and methods and accessory methods described herein are representative of embodiments of the present invention; they are not intended to limit the scope of the invention. Thus, additional embodiments are within the scope of the invention and within the following claims. The scope of the invention should be determined by the appended claims and their equivalents, rather than by the specific examples given.

### EXAMPLES

### Example 1

### Bacterial Strains, Plasmids and Growth Conditions

E. coli strain JM109 (21) was used for the maintenance of plasmids. E. coli strains were grown in Luria-Bertani (LB) broth (Difco) at 37° C. with agitation, or on LB agar plates supplemented with 1.5% agar, and where appropriate, with the following antibiotics: 100 μg/ml ampicillin, 25 μg/ml kanamycin, 150 μg/ml erythromycin or 10 μg/ml tetracycline. Meningococcal strains were grown on GC agar base (Oxoid) supplemented with 20 mM glucose, 0.43 μM thiamine pyrophosphate chloride, 6.8 mM glutamine and 12.4 μM Fe(NO3)₃ in 5% carbon dioxide atmosphere. Liquid cultures were vigorously aerated at 37° C. in GC broth with the same supplements and 0.51 M sodium bicarbonate (22). Where appropriate, media was supplemented with 7 μg/ml erythromycin, 1 μg/ml tetracycline or 40 μg/ml kanamycin.

### Example 2

### DNA Manipulations

All DNA manipulations were performed by standard methods as described previously (23). Plasmid DNA was purified using the Hi PURE Plasmid Isolation kit from Roche Diagnostics (Indianapolis, Ind.). Restriction endonucleases were purchased from New England Biolabs (Beverly, Mass.). DNA sequencing was performed with the BigDye Terminator V3.1 cycle sequencing kit (Applied Biosystems, Foster City, Calif.) and analyzed on an Applied Biosystems model 3730 DNA analyzer. Oligonucleotide primers were synthesized on an Applied Biosystems 394 oligonucleotide synthesizer. Competent E. coli JM109 was prepared by the method described by Chung et al. (24). Amino acid sequences were aligned either by CLUSTALW (on the internet, address ebi.ac.uk/clustalw/#) (25) or Blast 2 sequences (see the NCBI site on the internet: website ncbi.nlm.nih.gov/blast/bl2seq/wblast2.cgi) (26).

### Example 3

### Inactivation of the lipooligosaccharide O-acetyltransferase (lot3)

The lot3 gene in N. meningitidis strain NMB was inactivated by the incorporation of a lot3::tetM mutagenic cassette. To construct the lot3::tetM cassette, an internal fragment of lot3 (formerly designated NMA2202 in N. meningitidis strain Z2491) was amplified using the primer pair DAP379 (5′-gtctcggtcgccgtaaagatagc-3′, SEQ ID NO:1) and DAP377 (5′-gcagtagaggaacagtattacctcc-3′, SEQ ID NO:2) (FIG. 2). The resulting 1.5 kb PCR product was treated with T4 DNA polymerase and ligated into pHSG576 (27) digested with SmaI to create pJKD2709. The tetM cassette was released from pJKD2401 by EcoRV digestion and ligated to SmaI digested pJKD2709. Those transformants which were tetracycline and chloramphenicol resistant were assessed by colony PCR for the correct insertion of the tetM cassette into pJKD2709, and were designated pJKD2710.

To create NMBlot3, pJKD2710 was transformed in N. meningitidis strain NMB using the protocol of Janik et al. (28) and transformants were identified by resistance to tetracycline. To ensure these transformants contained the lot3::tetM cassette in the correct chromosomal location, the lot3 locus was PCR amplified with DAP371 (5′-CGATTTGTCGCGGAAAGAAACCG-3′, SEQ ID NO:3) and DAP372 (5′-gaagccaaagccaaattgcttgagc-3′, SEQ ID NO:4), located outside the lot3 open reading frame and the correct mutants were designated NMBlot3 (JKD5172). To create a meningococcal double mutant containing inactivated lot3 and IgtG, the IgtG::kan cassette was introduced into NMBlot3 by transformation with pCK49 (29), to create the strain designated NMBlot3/IgtG (JKD5173).

### Example 4

### Isolation of Oligosaccharides

The LOS preparations, prepared as previously described (30), were washed three times with 9:1 ethanol/water (v/v) mixture to remove contaminating phospholipids. The washed LOS preparations were suspended in water and lyophilized. Samples were then subjected to mild acid hydrolysis in 1% aqueous HOAc (v/v) for 2.5 h at 100° C. with constant stirring. The lipid A precipitate that formed during hydrolysis was collected by centrifugation at 3000×g for 15 min at 4° C., and supernatants containing the released oligosaccharides (OSs) were decanted and lyophilized. The lyophilized OSs were further purified by gel-filtration chromatography using Bio-Gel P-4 (Bio-Rad) column (120×1 cm) and water as eluent.

### Example 5

### Glycosyl Composition and Linkage Analysis of the Oligosaccharides

Glycosyl compositional analysis was performed by gas chromatography-mass spectrometry (GLC-MS) of trimethylsilyl (TMS) methyl glycosides with myoinositol used as an internal standard (31). The samples were methanolyzed with methanolic 1 M HCl at 80° C. for 18 h. The released monosaccharides were dried under a stream of dry air and acetylated with 3:1:1 methanol/pyridine/acetic anhydride (v/v/v) at 100° C. for 1 h. After cooling, samples were dried-down and trimethylsilylated with Tri-sil reagent (Pierce) for 30 min at 80° C. The resulting TMS derivatives were analyzed by GLC-MS, on Hewlett-Packard HP5890/HP5970 MSD gas chromatograph/mass spectrometer equipped with Supelco DB-1 fused silica capillary column (30 m×0.25 mm I.D.) with helium as the carrier gas.

Linkage analyses were carried out by the slurry NaOH method modified from that of Ciucanu and Kerek (32). Samples were dissolved in 0.5 mL dimethyl sulfoxide (DMSO) by stirring overnight at room temperature under a N₂ atmosphere. After dissolution, a freshly prepared slurry of NaOH in DMSO was added (0.5 mL) and the reaction mixture was stirred for 2 h at room temperature. Methylation was performed by the sequential addition of iodomethane (250 μL followed by 100 μL) at 30 min intervals. The permethylated monosaccharide was extracted into the organic phase (dichloromethane) after partitioning the reaction mixture between water and dichloromethane. The organic phase was then removed by evaporation under a stream of N₂. The permethylated OS was further purified using a Sep-Pak C18 cartridge to remove any remaining DMSO. The permethylated OS was hydrolyzed with 4 M TFA (100° C., 6 h), reduced with NaBH₄, acetylated and the resulting partially methylated alditol acetates (PMAAs) were analyzed by GLC/MS using a SPB capillary column (25 m×0.25 mm, from Supelco) and DB-1 capillary column.

### Example 6

### Mass Spectrometry

Oligosaccharides were analyzed by matrix-assisted laser desorption ionization time of flight mass spectrometry (MALDI-TOF MS) using a 4700 Proteomics Analyzer instrument (Applied Biosystems) in reflectron mode. The OS samples were dissolved in water (1 mg/mL) and mixed in a 1:1 (v/v) ratio with 0.5 M 2,5-dihydroxybenzoic acid (DHB) in methanol matrix solution. Spectra were acquired in both the positive and negative acquisition modes.

### Example 7

### NMR Spectroscopy

NMR spectra were collected on Varian Inova500 and 600 spectrometers using standard software supplied by Varian. The samples were exchanged several times with D₂O (99.8% Aldrich), and final measurements were made in 0.5 mL D₂O solutions (100% D; Cambridge Isotope Laboratories) at 25° C. Proton NMR spectra were measured at 600 MHz using spectral width of 8 kHz and the data were processed with HOD signal referenced at 4.78 ppm on proton scale. The correlated spectroscopy (gCOSY) spectra were measured over a spectral width of 2.25 kHz using a dataset of (t₁×t₂) of 256×2048 points with 16 scans. The total correlated spectroscopy (TOCSY) spectra were collected using the same sized data set with 32 scans with a mixing time of 80 msec. For the heteronuclear single quantum coherence (HSQC) experiment, the spectral widths in the proton and carbon dimensions were 2.2 and 13.9 kHz, respectively, and 96 scans were acquired. ¹D ³¹P NMR spectra were done at pH 7.0 using a Varian Inova-500 instrument with a broadband probe adjusted to 202.38 MHz. Proton decoupled ³¹P spectra were acquired with spectral width of 10 kHz calibrated with phosphoric acid (85%) as the external standard (δp=0.0 ppm). The 2D proton detected ¹H-³¹P heteronuclear multiple bond quantum coherence (HMQC) and HMQC-TOCSY experiments were performed using the standard pulse sequence supplied by Varian. The HMQC spectrum was collected using a data set of 128×2048 (t₁×t₂) points with total of 32 scans. The measurements of HMQC and HMQC-TOCSY spectra were done using J_{H,P} coupling value of 12.0 Hz. The mixing time used for HMQC-TOCSY spectrum was 60 msec and total of 64 scans were collected. The spectral width in ³¹P-¹H HMQC and HMQC-TOCSY experiments were set to 5 kHz in phosphorus dimension and 2.25 kHz in proton dimension respectively.

### Example 8

### PCR to Determine Presence or Absence of the Lot Gene

Chromosomal DNA of representative immunotyped stains L1-L12 were used as the template for PCR amplification of lot. Primers DAP 371 (5′-cgatttgtcgcggaaagaaaccg-3′, SEQ ID NO:3) and DAP 372 (5′-gaagccaaagccaaattgcttgagc-3′, SEQ ID NO:4) and an extended PCR cycle with a 50° C. annealing temperature and 4 minute auto-extend were used to amplify the gene, which is expected to be 2 Kb. All of the 12 representative strains contained lot. Both strands for each PCR product were sequenced and a consensus was generated (See below).

Immunotyping strains are shown in Table 11. All strains were obtained from Dr Wendell Zollinger at the Walter Reed Army Institute of Research.

TABLE 1
Expression matrix for meningococcal LOS immunotypes.

| | | LOS biosynthesis genes* | | | | | |
|---|---|---|---|---|---|---|---|
| Strain | Immunotype | **IgtA | **IgtC | §**IgtG | †Ipt-3 | §Ipt-6 | ‡Iot3 |
| 126E | L1 | P“−“ | P”+” | P”−“ or A | P | A | P”+” |
| NMB | L2 | P“+” | A | P”+” | Por A | P | P”+” |
| MC58 | L3 | P“+” | A | P”−“ or A | P | A | P”−” |
| 89I | L4 | P“+” | A | P”−“ or A | A | P | P”+” |
| M981 | L5 | P”+” | A | P”+” | A | A | P”+” |
| M922 | L6 | P”+” | A | P”−“ or A | A | P | P”+” |

*“A” indicates absent, “P” indicates present, “−“ indicates phase off, “+” indicates phase on.
**The presence or absence and phase of expression for these genes in the representative isolates has been established by Jennings et al (15).
§IgtG and Ipt-6 are present on two separate islets that are carried separately or together (18).
†Ipt-3 is carried by most isolates but may contain internal deletions which inactivate the locus (17).
‡The presence of Iot3 has been established in representative meningococcal strains expressing all twelve immunotypes (7). O-acetylation of the chemical structures for immunotypes L1, L2, L4, L5, and L6 has been established (see FIG. 1 for references).

TABLE 2
Proton chemical shift values for the
OS from NMB LOS (partial assignment)

| Residue | H1 | H2 | H3 | H4 | H5 | H6a, b | H7a, b |
|---|---|---|---|---|---|---|---|
| α-L-D-HepII (A) | 5.77 | 4.25 | 4.23 | 4.17 | 3.76 | 4.57 | 3.83, |
| | | | | | | | 3.90 |
| α-D-Glc (B) | 5.43 | 3.6 | 3.72 | 3.48 | n.d. | n.d. | n.d. |
| α-D-GlcNAc (C) | 5.28 | 4.19 | 5.17* | 3.68 | n.d. | n.d. | n.d. |
| α-L-D-HepI (D) | 5.09 | 4.18 | 4.16 | 4.08 | n.d. | n.d. | n.d. |
| β-D-GlcNAc (E) | 4.72 | 3.81 | 3.74 | 3.59 | n.d. | n.d. | n.d. |
| β-D-Glc (F) | 4.56 | 3.44 | 3.64 | 3.56 | n.d. | n.d. | n.d. |
| β-D-Gal (G) | 4.48 | 3.55 | 3.67 | 3.93 | n.d. | n.d. | n.d. |
| β-D-Gal (H) | 4.43 | 3.59 | 3.75 | 4.16 | n.d. | n.d. | n.d. |

*Indicates the position of O-acetyl group; n.d. = not detected. The residues are labeled A-H in descending order of their chemical shifts. The arrangement of these glycosyl residues are as shown on the structures given in FIG. 3.

TABLE 3
Proton chemical shift values of the OS
from NMBIot3 OS (partial assignment)

| Residue | H1 | H2 | H3 | H4 | H5 | H6a, b | H7a, b |
|---|---|---|---|---|---|---|---|
| α-L-D-HepII (A) | 5.71 | 4.18 | 4.21 | 4.16 | 3.73 | 4.56 | 3.74, |
| | | | | | | | 3.82 |
| α-D-Glc (B) | 5.41 | 3.58 | 3.72 | n.d. | n.d. | n.d. | n.d. |
| α-D-GlcNAc (C) | 5.18 | 3.89 | 3.85 | 3.48 | 3.86 | 3.9 | n.d. |
| α-L-D-HepI (D) | 5.09 | 4.17 | 4.11 | n.d. | n.d. | n.d. | n.d. |
| β-D-GlcNAc (E) | 4.72 | 3.82 | 3.76 | 3.59 | n.d. | n.d. | n.d. |
| β-D-Glc (F) | 4.56 | 3.44 | 3.65 | 3.56 | n.d. | n.d. | n.d. |
| β-D-Gal (G) | 4.48 | 3.55 | 3.68 | 3.94 | 3.56 | 3.44 | n.d. |
| β-D-Gal (H) | 4.46 | 3.59 | 3.75 | 4.16 | 3.92 | | n.d. |

*Indicates the position of O-acetyl group; n.d. = not detected.

TABLE 4A

L1 lot (SEQ ID NO: 5)
MQAVRYRPEIDGLRAVAVLSVIIFHLNNRWLPGGFLGVDIFFVISGFLIT
GIILSEIQNGSFSFRDFYTRRIKRIYPAFIAAVSLASVIASQIFLYEDFN
QMRKTVELSAVFLSNIYLGFQQGYFDLSADENPVLHIWSLAVEEQYYLLY
PLLLIFCCKKTKSLRVLRNISIILFLILTATSFLPSGFYTDILNQPNTYY
LSTLRFPELLAGSLLAVYGQTQNGRRQTANGKRQLLSSLCFGALLACLFV
IDKHNPFIPGMTLLLPCLLTALLIRSMQYGTLPTRILSASPIVFVGKISY
SLYLYHWIFIAFAHYITGDKQLGLPAVSAVAALTAGFSLLSYYLIEQPLR
KRKMTFKKAFFCLYLAPSLILVGYNLYARGILKQEHLRPLPGAPLAAENH
FPETVLTLGDSHAGHLRGFLDYVGSREGWKAKILSLDSECLVWVDEKLAD
NPLCRKYRDEVEKAEAVFIAQFYDLRMGGQPVPRFEAQSFLIPGFPARFR
ETVKRIAAVKPVYVFANNTSISRSPLREEKLKRFAANQYLRPIQAMGDIG
KSNQAVFDLVKDIPNVHWVDAQKYLPKNTVEIHGRYLYGDQDHLTYFGSY
YMGREFHKHERLLKSSRDGALQ

TABLE 4B

L2 lot (SEQ ID NO: 6)
MQAVRYRPEIDGLRAVAVLSVIIFHLNNRWLPGGFLGVDIFFVISGFLIT
GIILSEIQNGSFSFRDFYTRRIKRIYPAFIAAVSLASVIASQIFLYEDFN
QMRKTVELSAVFLSNIYLGFQQGYFDLSADENPVLHIWSLAVEEQYYLLY
PLLLIFCCKKTKSLRVLRNISIILFLILTASSFLPSGFYTDILNQPNTYY
LSTLRFPELLAGSLLAVYGQTQNGRRQTANGKRQLLSSLCFGALLACLFV
IDKHNPFIPGMTLLLPCLLTALLIRSMQYGTLPTRILSASPIVFVGKISY
SLYLYHWIFIAFAHYITGDKQLGLPAVSAVAALTAGFSLLSYYLIEQPLR
KRKMTFKKAFFCLYLAPSLILVGYNLYARGILKQEHLRPLPGAPLAAENH
FPETVLTLGDSHAGHLRGFLDYVGSREGWKAKILSLDSECLVWVDEKLAD
NPLCRKYRDEVEKAEAVFIAQFYDLRMGGQPVPRFEAQSFLIPGFQARFR
ETVKRIAAVKPVYVFANNTSISRSPLREEKLKRFAANQYLRPIQAMGDIG
KSNQAVFDLVKDIPNVHWVDAQKYLPKNTVEIHGCYLYGDQDHLTYFGSY
YMGREFHKHERLLKSSRDGALQ

TABLE 4C

L3 lot (SEQ ID NO: 7)
MQAVRYRPEIDGLRAVAVLSVIIFHLNNRWLPGGFLGVDIFFVISGFLIT
GIILSEIQNGSFSFRDFYTRRIKRIYPAFIAAVSLASVIASQIFLYEDFN
QMRKTVELSAVFLSNIYLGFQQGYFDLSADENPVLHIWSLAVEEQYYLLY
PLLLIFCCKKTKSLRVLRNISIILFLILTATSFLPSRFYTDILNQPNTYY
LSTLRFPELLAGSLLAVYGQTQNGRRQTASGKRQLLSLLCFGALLACLFV
IDKHNPFIPGMTLLLPCLLTALLIRSMQYGTLPTRILSASPIVFVGKISY
SLYLYHWIFIAFAHYITGDKQLGLPAVSAVAALTAGFSLLSYYLIEQPLR
KRKMTFKKAFFCLYLAPSLILVGYNLYARGILKQEHLRPLPGAPLAAENH
FPETVLTLGDSHAGHLRGFLDYVGSREGWKAKILSLDSECLVWVDTTLAD
NPLCRKYRDEVEKAEAVFIAQFYDLRMGGQPVPRFEAQSFLIPGFQARFR
ETVKRIAAVKPVYVFANNTSISRSPLREEKLKRFAANQYLRPIQAMGDIG
KSNQAVFDLVKDIPNVHWVDAQKYLPKNTVEIHGRYLYGDQDHLTYFGSY
YMGREFHKHERLLKSSRGGALQ.

TABLE 4D

L4 lot (SEQ ID NO: 8)
MQAVRYRPEIDGLRAVAVLSVIIFHLNNRWLPGGFLGVDIFFVISGFLIT
GIILSEIQNGSFSFRDFYTRRIKRIYPAFIAAVSLASVIASQIFLYEDFN
QMRKTVELSAVFLSNIYLGFQQGYFDLSADENPVLHIWSLAVEEQYYLLY
PLLLIFCCKKTKSLRVLRNISIILFLILTASSFLPSGFYTDILNQPNTYY
LSTLRFPELLAGSLLAVYGQTQNGRRQTANGKRQLLSSLCFGALLACLFV
IDKHNPFIPGMTLLLPCLLTALLIRSMQYGTLPTRILSASPIVFVGKISY
SLYLYHWIFIAFAHYITGDKQLGLPAVSAVAALTAGFSLLSYYLIEQPLR
KRKMTFKKAFFCLYLAPSLILVGYNLYARGILKQEHLRPLPGAPLAAENH
FPETVLTLGDSHAGHLRGFLDYVGSREGWKAKILSLDSECLVWVDEKLAD
NPLCRKYRDEVEKAEAVFIAQFYDLRMGGQPVPRFEAQSFLIPGFQARFR
ETVKRIAAVKPVYVFANNTSISRSPLREEKLKRFAANQYLRPIQAMGDIG
KSNQAVFDLVKDIPNVHWVDAQKYLPKNTVEIHGCYLYGDQDHLTYFGSY
YMGREFHKHERLLKSSRDGALQ

TABLE 4E

L5 lot (SEQ ID NO: 9)
MQAVRYRPEIDGLRAVAVLSVMIFHLNDRWLPGGFLGVDIFFVISGFLIT
GIILSEIQNGSFSFRDFYTRRIKRIYPAFIAAVSLASVIASQIFLYEDFN
QMRKTVELSAVFLSNIYLGFQQGYFDLSADENPVLHIWSLAVEEQYYLLY
PLLLIFCCKKTKSLRVLRNISIILFLILTATSFLPSGFYTDILNQPNTYY
LSTLRFPELLAGSLLAVYGQTQNGRRQTANGKRQLLSSLCFGALLACLFV
IDKHNPFIPGMTLLLPCLLTALLIRSMQYGTLPTRILSASPIVFVGKISY
SLYLYHWIFIAFAHYITGDKQLGLPAVSAVAALTAGFSLLSYYLIEQPLR
KRKMTFKKAFFCLYLAPSLILVGYNLYARGILKQEHLRPLPGAPLAAENH
FPETVLTLGDSHAGHLRGFLDYVGSREGWKAKILSLDSECLVWVDEKLAD
NPLCRKYRDEVEKAEAVFIAQFYDLRMGGQPVPRFEAQSFLIPGFPARFR
ETVKRIAAVKPVYVFANNTSISRSPLREEKLKRFAANQYLRPIQAMGDIG
KSNQAVFDLIKDIPNVHWVDAQKYLPKNTVEIHGRYLYGDQDHLTYFGSY
YMGREFHKHERLLKSSRGGALQ

TABLE 4F

L6 lot (SEQ ID NO: 10)
MQAVRYRPEIDGLRAVAVLSVIIFHLNNRWLPGGFLGVDIFFVISGFLIT
GIILSEIQNGSFSFRDFYTRRIKRIYPAFIAAVSLASVIASQIFLYEDFN
QMRKTVELSAVFLSNIYLGFQQGYFDLSADENPVLHIWSLAVEEQYYLLY
PLLLIFCCKKTKSLRVLRNISIILFLILTASSFLPSGFYTDILNQPNTYY
LSTLRFPELLAGSLLAVYGQTQNGRRQTANGKRQLLSSLCFGALLACLFV
IDKHNPFIPGMTLLLPCLLTALLIRSMQYGTLPTRILSASPIVFVGKISY
SLYLYHWIFIAFAHYITGDKQLGLPAVSAVAALTAGFSLLSYYLIEQPLR
KRKMTFKKAFFCLYLAPSLILVGYNLYARGILKQEHLRPLPGAPLAAENH
FPETVLTLGDSHAGHLRGFLDYVGSREGWKAKILSLDSECLVWVDEKLAD
NPLCRKYRDEVEKAEAVFIAQFYDLRMGGQPVPRFEAQSFLIPGFQARFR
ETVKRIAAVKPVYVFANNTSISRSPLREEKLKRFAANQYLRPIQAMGDIG
KSNQAVFDLVKDIPNVHWVDAQKYLPKNTVEIHGCYLYGDQDHLTYFGSY
YMGREFHKHERLLKSSRDGALQ

TABLE 4G

L7 lot (SEQ ID NO: 11)
MQTVRYRPEIDGLRAVAVLSVIIFHLNNRWLPGGFLGVDIFFVISGFLIT
GIILSEIQNGSFSFRDFYTRRIKRIYPAFIAAVSLASVIASQIFLYEDFN
QMRKTVELSAVFLSNIYLGFQQGYFDLSADENPVLHIWSLAVEEQYYLLY
PLLLIFCCKKTKSLRVLCHISIILFLILTASSFLPSGFYTDILNQPNTYY
LSTLRFPELLAGSLLAVYGQTQNGRRQTANGKRQLLSSLCFGALLACLFV
IDKHNPFIPGMTLLLPCLLTALLIRSMQYGTLPTRILSASPIVFVGKISY
SLYLYHWIFIAFAHYITGDKQLGLPAVSAVAALTAGFSLLSYYLIEQPLR
KRKMTFKKAFFCLYLAPSLILVGYNLYARGILKQEHLRPLPGAPLAAENH
FPETVLTLGDSHAGHLRGFLDYVGSREGWKAKILSLDSECLVWVDEKLAD
NPLCRKYRDEVEKAEAVFIAQFYDLRMGGQPVPRFEAQSFLIPGFPARFR
ETVKRIAAVKPVYVFANNTSISRSPLREEKLKRFAANQYLRPIQAMGDIG
KSNQAVFDLIKDIPNVHWVDAQKYLPKNTVEIYGRYLYGDQDHLTYFGSY
YMGREFHKHESLLKHSHGNALQ

TABLE 4H

L8 lot (SEQ ID NO: 12)
MQAVRYRPEIDGLRAVAVLSVIIFHLNNRWLPGGFLGVDIFFVISGFLIT
GIILSEIQNGSFSFRDFYTRRIKRIYPAFIAAVSLASVIASQIFLYEDFN
QMRKTGEFSAVFLSNIYLGFQQGYFDLSADENPVLHIWSLAVEEQYYLLY
PLLLIFCCKKTKSLRVLRNISIILFLILTATSFLPSGFYTDILNQPNTYY
LSTLRFPELLAGSLLAVYGQTQNGRRQTANGKRQLLSSLCFGALLACLFV
IDKHNPFIPGMTLLLPCLLTALLIRSMQYGTLPTRILSASSIVFVGKISY
SLYLYHWIFIAFAHYITGDKQLGLPAVSAVAALTAGFSLLSYYLIEQPLR
KRKMTFKKAFFCLYLAPSLILVGYNLYARGILKQEHLRPLPGAPLAAENH
FPETVLTLGDSHAGHLRGFLDYVGSREGWKAKILSLDSECLVWVDEKLAD
NPLCRKYRDEVEKAEAVFIAQFYDLRMGGQPVPRFEAQSFLIPGFQARFR
ETVKRIAAVKPVYVFANNTSISRSPLREEKLKRFAANQYLRPIQAMGDIG
KSNQAVFDLIKDIPNVHWVDAQKYLPKNTVEIYGRYLYGDQDHLTYFGSY
YMGREFHKHERLLKSSRDGALQ

TABLE 4I

L9 lot (SEQ ID NO: 13)
MQAVRYRPEIDGLRAVAVLSVMIFHLNNRWLPGGFLGVDIFFVISGFLIT
GIILSEIQNGSFSFRDFYTRRIKRIYPAFIAAVSLASVIASQIFLYEDFN
QMRKTVELSAVFLSNIYLGFQQGYFDLSADENPVLHIWSLAVEEQYYLLY
PLLLIFCCKKTKSLRVLRNISIILFLILTATSFLPSGFYTDILNQPNTYY
LSTLRFPELLAGSLLAVYGQTQNGRRQTANGKRQLLSSLCFGALLACLFV
IDKHNPFIPGMTLLLPCLLTALLIRSMQYGTLPTRILSASPIVFVGKISY
SLYLYHWIFIAFAHYITGDKQLGLPAVSAVAALTAGFSLLSYYLIEQPLR
KRKMTFKKAFFCLYLAPSLILVGYNLYARGILKQEHLRPLPGAPLAAENH
FPETVLTLGDSHAGHLRGFLDYVGSREGWKAKILSLDSECLVWVDEKLAD
NPLCRKYRDEVEKAEAVFIAQFYDLRMGGQPVPRFEAQSFLIPGFPARFR
ETVKRIAAVKPVYVFANNTSISRSPLREEKLKRFAANQYLRPIQAMGDIG
KSNQAVFDLIKDIPNVHWVDAQKYLPKNTVEIYGRYLYGDQDHLTYFGSY
YMGREFHKHERLLKSSRDGALQ

TABLE 4J

L10 lot (SEQ ID NO: 14)
MQAVRYRPEIDGLRAVAVLSVMIFHLNNRWLPGGFLGVDIFFVISGFLIT
GIILSEIQNGSFSFRDFYTRRIKRIYPAFIAAVSLASVIASQIFLYEDFN
QMRKTVELSAVFLSNIYLGFQQGYFDLSADENPVLHIWSLAVEEQYYLLY
PLLLIFCCKKTKSLRVLRNISIILFLILTATSFLPSGFYTDILNQPNTYY
LSTLRFPELLAGSLLAVYGQTQNGRRQTANGKRQLLSSLCFGALLACLFV
IDKHNPFIPGMTLLLPCLLTALLIRSMQYGTLPTRILSASPIVFVGKISY
SLYLYHWIFIAFAHYITGDKQLGLPAVSAVAALTAGFSLLSYYLIEQPLR
KRKMTFKKAFFCLYLAPSLILVGYNLYARGILKQEHLRPLPGAPLAAENH
FPETVLTLGDSHAGHLRGFLDYVGSREGWKAKILSLDSECLVWVDEKLAD
NPLCRKYRDEVEKAEAVFIAQFYDLRMGGQPVPRFEAQSFLIPGFPARFR
ETVKRIAAVKPVYVFANNTSISRSPLREEKLKRFAANQYLRPIQAMGDIG
KSNQAVFDLIKDIPNVHWVDAQKYLPKNTVEIYGRYLYGDQDHLTYFGSY
YMGREFHKHERLLKSSRDGALQ

TABLE 4K

L11 lot (SEQ ID NO: 15)
MQAVRYRPEIDGLRAVAVLSVMIFHLNNRWLPGGFLGVDIFFVISGFLIT
GIILSEIQ
NGSFSFRDFYTRRIKRIYPAFIAAVSLASVIASQIFLYEDFNQMRKTVEL
SAVFLSNIYLGFQQGYFDLSADENPVLHIWSLAVEEQYYLLYPLLLIFCC
KKTKSLRVLRNISIILFLILTATSFLPSGFYTDILNQPNTYYLSTLRFPE
LLAGSLLAVYGQTQNGRRQTANGKRQLLSSLCFGALLACLFVIDKHNPFI
PGMTLLLPCLLTALLIRSMQYGTLPTRILSASPIVFVGKISYSLYLYHWI
FIAFAHYITGDKQLGLPAVSAVAALTAGFSLLSYYLIEQPLRKRKMTFKK
AFFCLYLAPSLILVGYNLYARGILKQEHLRPLPGAPLAAENHFPETVLTL
GDSHAGHLRGFLDYVGSREGWKAKILSLDSECLVWVDEKLADNPLCRKYR
DEVEKAEAVFIAQFYDLRMGGQPVPRFEAQSFLIPGFPARFRETVKRIAA
VKPVYVFANNTSISRSPLREEKLKRFAANQYLRPIQAMGDIGKSNQAVFD
LIKDIPNVHWVDAQKYLPKNTVEIYGRYLYGDQDHLTYFGSYYMGREFHK
HERLLKSSRDGALQ

TABLE 4L

L12 lot (SEQ ID NO: 16)
MQAVRYRPEIDGLRAVAVLSVMIFHLNNRWLPGGFLGVDIFFVISGFLIT
GIILSEIQNGSFSFRDFYTRRIKRIYPAFIAAVSLASVIASQIFLYEDFN
QMRKTVELSAVFLSNIYLGFQQGYFDLSADENPVLHIWSLAVEEQYYLLY
PLLLIFCCKKTKSLRVLRNISIILFLILTATSFLPSGFYTDILNQPNTYY
LSTLRFPELLAGSLLAVYGQTQNGRRQTANGKRQLLSSLCFGALLACLFV
IDKHNPFIPGMTLLLPCLLTALLIRSMQYGTLPTRILSASPIVFVGKISY
SLYLYHWIFIAFAHYITGDKQLGLPAVSAVAALTAGFSLLSYYLIEQPLR
KRKMTFKKAFFCLYLAPSLILVGYNLYARGILKQEHLRPLPGAPLAAENH
FPETVLTLGDSHAGHLRGFLDYVGSREGWKAKILSLDSECLVWVDEKLAD
NPLCRKYRDEVEKAEAVFIAQFYDLRMGGQPVPRFEAQSFLIPGFPARFR
ETVKRIAAVKPVYVFANNTSISRSPLREEKLKRFAANQYLRPIQAMGDIG
KSNQAVFDLIKDIPNVHWVDAQKYLPKNTVEIYGRYLYGDQDHLTYFGSY
YMGREFHKHERLLKSSRDGALQ

TABLE 5

Results of CLUSTAL W (1.82) multiple sequence alignment for the
twelve O-acetyl transferase proteins in Table 4A-4L (SEQ ID NOs: 5-16,
respectively).

| | | |
|---|---|---|
| L2lot | MQAVRYRPEIDGLRAVAVLSVIIFHLNNRWLPGGFLGVDIFFVISGFLITGIILSEIQNG | 60 |
| L4 | MQAVRYRPEIDGLRAVAVLSVIIFHLNNRWLPGGFLGVDIFFVISGFLITGIILSEIQNG | 60 |
| L6lot | MQAVRYRPEIDGLRAVAVLSVIIFHLNNRWLPGGFLGVDIFFVISGFLITGIILSEIQNG | 60 |
| L3lot | MQAVRYRPEIDGLRAVAVLSVIIFHLNNRWLPGGFLGVDIFFVISGFLITGIILSEIQNG | 60 |
| L1lot | MQAVRYRPEIDGLRAVAVLSVIIFHLNNRWLPGGFLGVDIFFVISGFLITGIILSEIQNG | 60 |
| L8lot | MQAVRYRPEIDGLRAVAVLSVIIFHLNNRWLPGGFLGVDIFFVISGFLITGIILSEIQNG | 60 |
| L11lot | MQAVRYRPEIDGLRAVAVLSVMIFHLNNRWLPGGFLGVDIFFVISGFLITGIILSEIQNG | 60 |
| L12lot | MQAVRYRPEIDGLRAVAVLSVMIFHLNNRWLPGGFLGVDIFFVISGFLITGIILSEIQNG | 60 |
| L10lot | MQAVRYRPEIDGLRAVAVLSVMIFHLNNRWLPGGFLGVDIFFVISGFLITGIILSEIQNG | 60 |
| L9lot | MQAVRYRPEIDGLRAVAVLSVMIFHLNNRWLPGGFLGVDIFFVISGFLITGIILSEIQNG | 60 |
| L5lot | MQAVRYRPEIDGLRAVAVLSVMIFHLNDRWLPGGFLGVDIFFVISGFLITGIILSEIQNG | 60 |
| L7lot | MQTVRYRPEIDGLRAVAVLSVIIFHLNNRWLPGGFLGVDIFFVISGFLITGIILSEIQNG | 60 |
| | **:******************:*****:******************************** | |
| L2lot | SFSFRDFYTRRIKRIYPAFIAAVSLASVIASQIFLYEDFNQMRKTVELSAVFLSNIYLGF | 120 |
| L4 | SFSFRDFYTRRIKRIYPAFIAAVSLASVIASQIFLYEDFNQMRKTVELSAVFLSNIYLGF | 120 |
| L6lot | SFSFRDFYTRRIKRIYPAFIAAVSLASVIASQIFLYEDFNQMRKTVELSAVFLSNIYLGF | 120 |
| L3lot | SFSFRDFYTRRIKRIYPAFIAAVSLASVIASQIFLYEDFNQMRKTVELSAVFLSNIYLGF | 120 |
| L1lot | SFSFRDFYTRRIKRIYPAFIAAVSLASVIASQIFLYEDFNQMRKTVELSAVFLSNIYLGF | 120 |
| L8lot | SFSFRDFYTRRIKRIYPAFIAAVSLASVIASQIFLYEDFNQMRKTGEFSAVFLSNIYLGF | 120 |
| L11lot | SFSFRDFYTRRIKRIYPAFIAAVSLASVIASQIFLYEDFNQMRKTVELSAVFLSNIYLGF | 120 |
| L12lot | SFSFRDFYTRRIKRIYPAFIAAVSLASVIASQIFLYEDFNQMRKTVELSAVFLSNIYLGF | 120 |
| L10lot | SFSFRDFYTRRIKRIYPAFIAAVSLASVIASQIFLYEDFNQMRKTVELSAVFLSNIYLGF | 120 |
| L9lot | SFSFRDFYTRRIKRIYPAFIAAVSLASVIASQIFLYEDFNQMRKTVELSAVFLSNIYLGF | 120 |
| L5lot | SFSFRDFYTRRIKRIYPAFIAAVSLASVIASQIFLYEDFNQMRKTVELSAVFLSNIYLGF | 120 |
| L7lot | SFSFRDFYTRRIKRIYPAFIAAVSLASVIASQIFLYEDFNQMRKTVELSAVFLSNIYLGF | 120 |
| | ***********************************************:************ | |
| L2lot | QQGYFDLSADENPVLHIWSLAVEEQYYLLYPLLLIFCCKKTKSLRVLRNISIILFLILTA | 180 |
| L4 | QQGYFDLSADENPVLHIWSLAVEEQYYLLYPLLLIFCCKKTKSLRVLRNISIILFLILTA | 180 |
| L6lot | QQGYFDLSADENPVLHIWSLAVEEQYYLLYPLLLIFCCKKTKSLRVLRNISIILFLILTA | 180 |
| L3lot | QQGYFDLSADENPVLHIWSLAVEEQYYLLYPLLLIFCCKKTKSLRVLRNISIILFLILTA | 180 |
| L1lot | QQGYFDLSADENPVLHIWSLAVEEQYYLLYPLLLIFCCKKTKSLRVLRNISIILFLILTA | 180 |
| L8lot | QQGYFDLSADENPVLHIWSLAVEEQYYLLYPLLLIFCCKKTKSLRVLRNISIILFLILTA | 180 |
| L11lot | QQGYFDLSADENPVLHIWSLAVEEQYYLLYPLLLIFCCKKTKSLRVLRNISIILFLILTA | 180 |
| L12lot | QQGYFDLSADENPVLHIWSLAVEEQYYLLYPLLLIFCCKKTKSLRVLRNISIILFLILTA | 180 |
| L10lot | QQGYFDLSADENPVLHIWSLAVEEQYYLLYPLLLIFCCKKTKSLRVLRNISIILFLILTA | 180 |
| L9lot | QQGYFDLSADENPVLHIWSLAVEEQYYLLYPLLLIFCCKKTKSLRVLRNISIILFLILTA | 180 |
| L5lot | QQGYFDLSADENPVLHIWSLAVEEQYYLLYPLLLIFCCKKTKSLRVLRNISIILFLILTA | 180 |
| L7lot | QQGYFDLSADENPVLHIWSLAVEEQYYLLYPLLLIFCCKKTKSLRVLCHISIILFLILTA | 180 |
| | *********************************************** :*********** | |
| L2lot | SSFLPSGFYTDILNQPNTYYLSTLRFPELLAGSLLAVYGQTQNGRRQTANGKRQLLSSLC | 240 |
| L4 | SSFLPSGFYTDILNQPNTYYLSTLRFPELLAGSLLAVYGQTQNGRRQTANGKRQLLSSLC | 240 |
| L6lot | SSFLPSGFYTDILNQPNTYYLSTLRFPELLAGSLLAVYGQTQNGRRQTANGKRQLLSSLC | 240 |
| L3lot | TSFLPSRFYTDILNQPNTYYLSTLRFPELLAGSLLAVYGQTQNGRRQTASGKRQLLSLLC | 240 |
| L1lot | TSFLPSGFYTDILNQPNTYYLSTLRFPELLAGSLLAVYGQTQNGRRQTANGKRQLLSSLC | 240 |
| L8lot | TSFLPSGFYTDILNQPNTYYLSTLRFPELLAGSLLAVYGQTQNGRRQTANGKRQLLSSLC | 240 |
| L11lot | TSFLPSGFYTDILNQPNTYYLSTLRFPELLAGSLLAVYGQTQNGRRQTANGKRQLLSSLC | 240 |
| L12lot | TSFLPSGFYTDILNQPNTYYLSTLRFPELLAGSLLAVYGQTQNGRRQTANGKRQLLSSLC | 240 |
| L10lot | TSFLPSGFYTDILNQPNTYYLSTLRFPELLAGSLLAVYGQTQNGRRQTANGKRQLLSSLC | 240 |
| L9lot | TSFLPSGFYTDILNQPNTYYLSTLRFPELLAGSLLAVYGQTQNGRRQTANGKRQLLSSLC | 240 |
| L5lot | TSFLPSGFYTDILNQPNTYYLSTLRFPELLAGSLLAVYGQTQNGRRQTANGKRQLLSSLC | 240 |
| L7lot | SSFLPSGFYTDILNQPNTYYLSTLRFPELLAGSLLAVYGQTQNGRRQTANGKRQLLSSLC | 240 |
| | :***** ******************************************.******* ** | |
| L2lot | FGALLACLFVIDKHNPFIPGMTLLLPCLLTALLIRSMQYGTLPTRILSASPIVFVGKISY | 300 |
| L4 | FGALLACLFVIDKHNPFIPGMTLLLPCLLTALLIRSMQYGTLPTRILSASPIVFVGKISY | 300 |
| L6lot | FGALLACLFVIDKHNPFIPGMTLLLPCLLTALLIRSMQYGTLPTRILSASPIVFVGKISY | 300 |
| L3lot | FGALLACLFVIDKHNPFIPGMTLLLPCLLTALLIRSMQYGTLPTRILSASPIVFVGKISY | 300 |
| L1lot | FGALLACLFVIDKHNPFIPGMTLLLPCLLTALLIRSMQYGTLPTRILSASPIVFVGKISY | 300 |
| L8lot | FGALLACLFVIDKHNPFIPGMTLLLPCLLTALLIRSMQYGTLPTRILSASSIVFVGKISY | 300 |
| L11lot | FGALLACLFVIDKHNPFIPGMTLLLPCLLTALLIRSMQYGTLPTRILSASPIVFVGKISY | 300 |
| L12lot | FGALLACLFVIDKHNPFIPGMTLLLPCLLTALLIRSMQYGTLPTRILSASPIVFVGKISY | 300 |
| L10lot | FGALLACLFVIDKHNPFIPGMTLLLPCLLTALLIRSMQYGTLPTRILSASPIVFVGKISY | 300 |
| L9lot | FGALLACLFVIDKHNPFIPGMTLLLPCLLTALLIRSMQYGTLPTRILSASPIVFVGKISY | 300 |
| L5lot | FGALLACLFVIDKHNPFIPGMTLLLPCLLTALLIRSMQYGTLPTRILSASPIVFVGKISY | 300 |
| L7lot | FGALLACLFVIDKHNPFIPGMTLLLPCLLTALLIRSMQYGTLPTRILSASPIVFVGKISY | 300 |
| | **************************************************.********* | |
| L2lot | SLYLYHWIFIAFAHYITGDKQLGLPAVSAVAALTAGFSLLSYYLIEQPLRKRKMTFKKAF | 360 |
| L4 | SLYLYHWIFIAFAHYITGDKQLGLPAVSAVAALTAGFSLLSYYLIEQPLRKRKMTFKKAF | 360 |
| L6lot | SLYLYHWIFIAFAHYITGDKQLGLPAVSAVAALTAGFSLLSYYLIEQPLRKRKMTFKKAF | 360 |
| L3lot | SLYLYHWIFIAFAHYITGDKQLGLPAVSAVAALTAGFSLLSYYLIEQPLRKRKMTFKKAF | 360 |
| L1lot | SLYLYHWIFIAFAHYITGDKQLGLPAVSAVAALTAGFSLLSYYLIEQPLRKRKMTFKKAF | 360 |
| L8lot | SLYLYHWIFIAFAHYITGDKQLGLPAVSAVAALTAGFSLLSYYLIEQPLRKRKMTFKKAF | 360 |
| L11lot | SLYLYHWIFIAFAHYITGDKQLGLPAVSAVAALTAGFSLLSYYLIEQPLRKRKMTFKKAF | 360 |
| L12lot | SLYLYHWIFIAFAHYITGDKQLGLPAVSAVAALTAGFSLLSYYLIEQPLRKRKMTFKKAF | 360 |
| L10lot | SLYLYHWIFIAFAHYITGDKQLGLPAVSAVAALTAGFSLLSYYLIEQPLRKRKMTFKKAF | 360 |
| L9lot | SLYLYHWIFIAFAHYITGDKQLGLPAVSAVAALTAGFSLLSYYLIEQPLRKRKMTFKKAF | 360 |
| L5lot | SLYLYHWIFIAFAHYITGDKQLGLPAVSAVAALTAGFSLLSYYLIEQPLRKRKMTFKKAF | 360 |
| L7lot | SLYLYHWIFIAFAHYITGDKQLGLPAVSAVAALTAGFSLLSYYLIEQPLRKRKMTFKKAF | 360 |
| | ************************************************************ | |
| L2lot | FCLYLAPSLILVGYNLYARGILKQEHLRPLPGAPLAAENHFPETVLTLGDSHAGHLRGFL | 420 |
| L4 | FCLYLAPSLILVGYNLYARGILKQEHLRPLPGAPLAAENHFPETVLTLGDSHAGHLRGFL | 420 |
| L6lot | FCLYLAPSLILVGYNLYARGILKQEHLRPLPGAPLAAENHFPETVLTLGDSHAGHLRGFL | 420 |
| L3lot | FCLYLAPSLILVGYNLYARGILKQEHLRPLPGAPLAAENHFPETVLTLGDSHAGHLRGFL | 420 |
| L1lot | FCLYLAPSLILVGYNLYARGILKQEHLRPLPGAPLAAENHFPETVLTLGDSHAGHLRGFL | 420 |
| L8lot | FCLYLAPSLILVGYNLYARGILKQEHLRPLPGAPLAAENHFPETVLTLGDSHAGHLRGFL | 420 |
| L11lot | FCLYLAPSLILVGYNLYARGILKQEHLRPLPGAPLAAENHFPETVLTLGDSHAGHLRGFL | 420 |
| L12lot | FCLYLAPSLILVGYNLYARGILKQEHLRPLPGAPLAAENHFPETVLTLGDSHAGHLRGFL | 420 |
| L10lot | FCLYLAPSLILVGYNLYARGILKQEHLRPLPGAPLAAENHFPETVLTLGDSHAGHLRGFL | 420 |
| L9lot | FCLYLAPSLILVGYNLYARGILKQEHLRPLPGAPLAAENHFPETVLTLGDSHAGHLRGFL | 420 |
| L5lot | FCLYLAPSLILVGYNLYARGILKQEHLRPLPGAPLAAENHFPETVLTLGDSHAGHLRGFL | 420 |
| L7lot | FCLYLAPSLILVGYNLYARGILKQEHLRPLPGAPLAAENHFPETVLTLGDSHAGHLRGFL | 420 |
| | ************************************************************ | |
| L2lot | DYVGSREGWKAKILSLDSECLVWVDEKLADNPLCRKYRDEVEKAEAVFIAQFYDLRMGGQ | 480 |
| L4 | DYVGSREGWKAKILSLDSECLVWVDEKLADNPLCRKYRDEVEKAEAVFIAQFYDLRMGGQ | 480 |
| L6lot | DYVGSREGWKAKILSLDSECLVWVDEKLADNPLCRKYRDEVEKAEAVFIAQFYDLRMGGQ | 480 |
| L3lot | DYVGSREGWKAKILSLDSECLVWVDTTLADNPLCRKYRDEVEKAEAVFIAQFYDLRMGGQ | 480 |
| L1lot | DYVGSREGWKAKILSLDSECLVWVDEKLADNPLCRKYRDEVEKAEAVFIAQFYDLRMGGQ | 480 |
| L8lot | DYVGSREGWKAKILSLDSECLVWVDEKLADNPLCRKYRDEVEKAEAVFIAQFYDLRMGGQ | 480 |
| L11lot | DYVGSREGWKAKILSLDSECLVWVDEKLADNPLCRKYRDEVEKAEAVFIAQFYDLRMGGQ | 480 |
| L12lot | DYVGSREGWKAKILSLDSECLVWVDEKLADNPLCRKYRDEVEKAEAVFIAQFYDLRMGGQ | 480 |
| L10lot | DYVGSREGWKAKILSLDSECLVWVDEKLADNPLCRKYRDEVEKAEAVFIAQFYDLRMGGQ | 480 |
| L9lot | DYVGSREGWKAKILSLDSECLVWVDEKLADNPLCRKYRDEVEKAEAVFIAQFYDLRMGGQ | 480 |
| L5lot | DYVGSREGWKAKILSLDSECLVWVDEKLADNPLCRKYRDEVEKAEAVFIAQFYDLRMGGQ | 480 |
| L7lot | DYVGSREGWKAKILSLDSECLVWVDEKLADNPLCRKYRDEVEKAEAVFIAQFYDLRMGGQ | 480 |
| | ************************* .********************************* | |
| L2lot | PVPRFEAQSFLIPGFQARFRETVKRIAAVKPVYVFANNTSISRSPLREEKLKRFAANQYL | 540 |
| L4 | PVPRFEAQSFLIPGFQARFRETVKRIAAVKPVYVFANNTSISRSPLREEKLKRFAANQYL | 540 |
| L6lot | PVPRFEAQSFLIPGFQARFRETVKRIAAVKPVYVFANNTSISRSPLREEKLKRFAANQYL | 540 |
| L3lot | PVPRFEAQSFLIPGFQARFRETVKRIAAVKPVYVFANNTSISRSPLREEKLKRFAANQYL | 540 |
| L1lot | PVPRFEAQSFLIPGFPARFRETVKRIAAVKPVYVFANNTSISRSPLREEKLKRFAANQYL | 540 |
| L8lot | PVPRFEAQSFLIPGFQARFRETVKRIAAVKPVYVFANNTSISRSPLREEKLKRFAANQYL | 540 |
| L11lot | PVPRFEAQSFLIPGFPARFRETVKRIAAVKPVYVFANNTSISRSPLREEKLKRFAANQYL | 540 |
| L12lot | PVPRFEAQSFLIPGFPARFRETVKRIAAVKPVYVFANNTSISRSPLREEKLKRFAANQYL | 540 |
| L10lot | PVPRFEAQSFLIPGFPARFRETVKRIAAVKPVYVFANNTSISRSPLREEKLKRFAANQYL | 540 |
| L9lot | PVPRFEAQSFLIPGFPARFRETVKRIAAVKPVYVFANNTSISRSPLREEKLKRFAANQYL | 540 |
| L5lot | PVPRFEAQSFLIPGFPARFRETVKRIAAVKPVYVFANNTSISRSPLREEKLKRFAANQYL | 540 |
| L7lot | PVPRFEAQSFLIPGFPARFRETVKRIAAVKPVYVFANNTSISRSPLREEKLKRFAANQYL | 540 |
| | *************** ******************************************** | |
| L2lot | RPIQAMGDIGKSNQAVFDLVKDIPNVHWVDAQKYLPKNTVEIHGCYLYGDQDHLTYFGSY | 600 |
| L4 | RPIQAMGDIGKSNQAVFDLVKDIPNVHWVDAQKYLPKNTVEIHGCYLYGDQDHLTYFGSY | 600 |
| L6lot | RPIQAMGDIGKSNQAVFDLVKDIPNVHWVDAQKYLPKNTVEIHGCYLYGDQDHLTYFGSY | 600 |
| L3lot | RPIQAMGDIGKSNQAVFDLVKDIPNVHWVDAQKYLPKNTVEIHGRYLYGDQDHLTYFGSY | 600 |
| L1lot | RPIQAMGDIGKSNQAVFDLVKDIPNVHWVDAQKYLPKNTVEIHGRYLYGDQDHLTYFGSY | 600 |
| L8lot | RPIQAMGDIGKSNQAVFDLIKDIPNVHWVDAQKYLPKNTVEIYGRYLYGDQDHLTYFGSY | 600 |
| L11lot | RPIQAMGDIGKSNQAVFDLIKDIPNVHWVDAQKYLPKNTVEIYGRYLYGDQDHLTYFGSY | 600 |
| L12lot | RPIQAMGDIGKSNQAVFDLIKDIPNVHWVDAQKYLPKNTVEIYGRYLYGDQDHLTYFGSY | 600 |
| L10lot | RPIQAMGDIGKSNQAVFDLIKDIPNVHWVDAQKYLPKNTVEIYGRYLYGDQDHLTYFGSY | 600 |
| L9lot | RPIQAMGDIGKSNQAVFDLIKDIPNVHWVDAQKYLPKNTVEIYGRYLYGDQDHLTYFGSY | 600 |
| L5lot | RPIQAMGDIGKSNQAVFDLIKDIPNVHWVDAQKYLPKNTVEIHGRYLYGDQDHLTYFGSY | 600 |
| L7lot | RPIQAMGDIGKSNQAVFDLIKDIPNVHWVDAQKYLPKNTVEIYGRYLYGDQDHLTYFGSY | 600 |
| | *******************:**********************:* *************** | |
| L2lot | YMGREFHKHERLLKSSRDGALQ | 622 |
| L4 | YMGREFHKHERLLKSSRDGALQ | 622 |
| L6lot | YMGREFHKHERLLKSSRDGALQ | 622 |
| L3lot | YMGREFHKHERLLKSSRGGALQ | 622 |
| L1lot | YMGREFHKHERLLKSSRDGALQ | 622 |
| L8lot | YMGREFHKHERLLKSSRDGALQ | 622 |
| L11lot | YMGREFHKHERLLKSSRDGALQ | 622 |
| L12lot | YMGREFHKHERLLKSSRDGALQ | 622 |
| L10lot | YMGREFHKHERLLKSSRDGALQ | 622 |
| L9lot | YMGREFHKHERLLKSSRDGALQ | 622 |
| L5lot | YMGREFHKHERLLKSSRGGALQ | 622 |
| L7lot | YMGREFHKHESLLKHSHGNALQ | 622 |
| | ********** *** *:..*** | |

TABLE 6A

L1 lot sequence (ATG translation start and TAG translation
termination codons underlined)(SEQ ID NO: 17)

AAACGGATTTGAGCGTTTACTGAAACCGATGCCGTCTGAACGCGCGTTCAGACGGCATT
TTTAAGATAACGGGACATACGGGGCGATATTTATGCAAGCTGTCCGATACAGGCCTGAA
ATTGACGGATTGCGGGCCGTTGCCGTGCTATCCGTCATTATTTTCCACCTGAATAACCG
CTGGCTGCCCGGAGGGTTTTTGGGGGTGGACATTTTCTTTGTCATCTCGGGATTCCTCA
TTACCGGCATCATTCTTTCTGAAATACAGAACGGTTCTTTTTCTTTCCGGGATTTTTAT
ACCCGCAGGATTAAGCGGATTTACCCTGCCTTTATTGCGGCCGTGTCGCTGGCTTCGGT
GATTGCCTCTCAAATCTTCCTTTACGAAGATTTCAACCAAATGCGGAAAACCGTGGAGC
TTTCTGCGGTTTTCTTGTCCAATATTTATCTGGGGTTTCAGCAGGGGTATTTCGATTTG
AGTGCCGACGAGAACCCCGTACTGCATATCTGGTCTTTGGCAGTAGAGGAACAGTATTA
CCTCCTGTATCCTCTTTTGCTGATATTTTGCTGCAAAAAAACAAAATCGCTACGGGTGC
TGCGTAACATCAGCATCATCCTATTTCTGATTTTGACTGCCACATCGTTTTTGCCAAGC
GGGTTTTATACCGATATTCTCAACCAACCCAATACTTATTACCTTTCGACACTGAGGTT
TCCCGAGCTGTTGGCAGGTTCGCTGCTGGCGGTTTACGGGCAAACGCAAAACGGCAGAC
GGCAAACAGCAAATGGAAAACGGCAGTTGCTTTCATCACTCTGCTTCGGCGCATTGCTT
GCCTGCCTGTTCGTGATTGACAAACACAATCCGTTTATCCCGGGAATGACCCTGCTCCT
TCCCTGCCTGCTGACGGCACTGCTTATCCGGAGTATGCAATACGGGACACTTCCGACCC
GCATCCTGTCGGCAAGCCCCATCGTATTTGTCGGCAAAATCTCTTATTCCCTATACCTG
TACCATTGGATTTTTATTGCTTTCGCCCATTACATTACAGGCGACAAACAGCTCGGACT
GCCTGCCGTATCGGCGGTTGCCGCGTTGACGGCCGGATTTTCCCTGTTGAGTTATTATT
TGATTGAACAGCCGCTTAGAAAACGGAAGATGACCTTCAAAAAGGCATTTTTCTGCCTC
TATCTCGCCCCGTCCCTGATACTTGTCGGTTACAACCTGTACGCAAGGGGGATATTGAA
ACAGGAACACCTCCGCCCGTTGCCCGGCGCGCCCCTTGCTGCGGAAAATCATTTTCCGG
AAACCGTCCTGACCCTCGGCGACTCGCACGCCGGACACCTGCGGGGGTTTCTGGATTAT
GTCGGCAGCCGGGAAGGGTGGAAAGCCAAAATCCTGTCCCTCGATTCGGAGTGTTTGGT
TTGGGTAGATGAGAAGCTGGCAGACAACCCGTTATGTCGAAAATACCGGGATGAAGTTG
AAAAAGCCGAAGCCGTTTTCATTGCCCAATTCTATGATTTGAGGATGGGCGGCCAGCCC
GTGCCGAGATTTGAAGCGCAATCCTTCCTAATACCCGGGTTCCCAGCCCGATTCAGGGA
AACCGTCAAAAGGATAGCCGCCGTCAAACCCGTCTATGTTTTTGCAAACAACACATCAA
TCAGCCGTTCGCCCCTGAGGGAGGAAAAATTGAAAAGATTTGCCGCAAACCAATATCTC
CGCCCCATTCAGGCTATGGGCGACATCGGCAAGAGCAATCAGGCGGTCTTTGATTTGGT
TAAAGATATCCCCAATGTGCATTGGGTGGACGCACAGAAATACCTGCCTAAAAACACGG
TCGAAATACACGGCCGCTATCTTTACGGCGACCAAGACCACCTGACCTATTTCGGTTCT
TATTATATGGGGCGGGAATTTCACAAACACGAACGCCTGCTTAAATCTTCTCGCGACGG
CGCATTGCAGTAGCCTGCCTTGCCGTCCGATATCGTTTGTGCCGCCGTTTGCCTTTCGG
GGCGGCGGCTTTTATAGTGGATTAACAAAAATCAGGACAAGGCAACGAAGCCGCAGACA
GTACAAATAGTACGGAACCGATTCACTTGGTGCTTCAGCACCTTAGAGAATCGTTCTCT
TTGAGCTAAGGCGAGGCAACGCCGTACTGGTTTTTGTTAATCCACTATATTTTGCCGTT
TTGAGGCCGGGGTCGGAATAACCGTTTTTTGATGATTTTCCCTCCCTGGCTGTGTCATC
AAAACCCCAATTGCCTTTCCAAACTCTCCACCAGATTGTCATCCAGTTTCAAAGCCTGC
GACAGGCGGGCGAGGAAGACGGTTTCTTTCCGGGAACGGAATCGAA

TABLE 6B

L2 lot consensus coding sequence; ATG translation start
codon and TAG translation termination codons underlined.
(SEQ ID NO: 18)

AACGGATTTGAGCGTTTACTGAAACCGATGCCGTCTGAACGCGCGTTCAGACGGCATTT
TTAAGATAACGGGACATACGGGGCGATATTTATGCAAGCTGTCCGATACAGGCCTGAAA
TTGACGGATTGCGGGCCGTTGCCGTGCTATCCGTCATTATTTTCCACCTGAATAACCGC
TGGCTGCCCGGAGGGTTTTTGGGGGTGGACATTTTCTTTGTCATCTCGGGATTCCTCAT
TACCGGCATCATTCTTTCTGAAATACAGAACGGTTCTTTTTCTTTCCGGGATTTTTATA
CCCGCAGGATTAAGCGGATTTACCCTGCCTTTATTGCGGCCGTGTCGCTGGCTTCGGTG
ATTGCCTCTCAAATCTTCCTTTACGAAGATTTCAACCAAATGCGGAAAACCGTGGAGCT
TTCTGCGGTTTTCTTGTCCAATATTTATCTGGGGTTTCAGCAGGGGTATTTCGATTTGA
GTGCCGACGAGAACCCCGTACTGCATATCTGGTCTTTGGCGGTAGAGGAACAGTATTAC
CTCCTGTATCCCCTTTTGCTGATATTTTGCTGCAAAAAAACCAAATCGCTACGGGTGCT
GCGTAACATCAGCATCATCCTGTTTTTGATTTTGACTGCCTCATCGTTTTTGCCAAGCG
GGTTTTATACCGACATCCTCAACCAACCCAATACTTATTACCTTTCGACACTGAGGTTT
CCCGAGCTGTTGGCAGGTTCGCTGCTGGCGGTTTACGGGCAAACGCAAAACGGCAGACG
GCAAACAGCAAATGGAAAACGGCAGTTGCTTTCATCACTCTGCTTCGGCGCATTGCTTG
CCTGCCTGTTCGTGATTGACAAACACAATCCGTTTATCCCGGGAATGACCCTGCTCCTT
CCCTGCCTGCTGACGGCACTGCTTATCCGGAGTATGCAATACGGGACACTTCCGACCCG
CATCCTGTCGGCAAGCCCCATCGTATTTGTCGGCAAAATCTCTTATTCCCTATACCTGT
ACCATTGGATTTTTATTGCTTTCGCCCATTACATTACAGGCGACAAACAGCTCGGACTG
CCTGCCGTATCGGCGGTTGCCGCGTTGACGGCCGGATTTTCCCTGTTGAGTTATTATTT
GATTGAACAGCCGCTTAGAAAACGGAAGATGACCTTCAAAAAGGCATTTTTCTGCCTCT
ATCTCGCCCCGTCCCTGATACTTGTCGGTTACAACCTGTACGCAAGGGGGATATTGAAA
CAGGAACACCTCCGCCCGTTGCCCGGCGCGCCCCTTGCTGCGGAAAATCATTTTCCGGA
AACCGTCCTGACCCTCGGCGACTCGCACGCCGGACACCTGCGGGGGTTTCTGGATTATG
TCGGCAGCCGGGAAGGGTGGAAAGCCAAAATCCTGTCCCTCGATTCGGAGTGTTTGGTT
TGGGTAGATGAGAAGCTGGCAGACAACCCGTTATGTCGAAAATACCGGGATGAAGTTGA
AAAAGCCGAAGCCGTTTTCATTGCCCAATTCTATGATTTGAGGATGGGCGGCCAGCCCG
TGCCGAGATTTGAAGCGCAATCCTTCCTAATACCCGGGTTCCAAGCCCGATTCAGGGAA
ACCGTCAAAAGGATAGCCGCCGTCAAACCCGTCTATGTTTTTGCAAACAACACATCAAT
CAGCCGTTCGCCCCTGAGGGAGGAAAAATTGAAAAGATTTGCCGCAAACCAATATCTCC
GCCCCATTCAGGCTATGGGCGACATCGGCAAGAGCAATCAGGCGGTCTTTGATTTGGTT
AAAGATATCCCCAATGTGCATTGGGTGGACGCACAGAAATACCTGCCTAAAAACACGGT
CGAAATACACGGCTGCTATCTTTACGGCGACCAAGACCACCTGACCTATTTCGGTTCTT
ATTATATGGGGCGGGAATTTCACAAACACGAACGCCTGCTTAAATCTTCTCGCGACGGC
GCATTGCAGTAGCCTGCCTTGCCGTCCGATATCGTTTGTGCCGCCGTTTGCCTTTCGGG
GCGGCGGCTTTTATAGTGGATTAACAAAAATCAGGACAAGGCAACGAAGCCGCAGACAG
TACAAATAGTACGGAACCGATTCACTTGGTGCTTCAGCACCTTAGAGAATCGTTCTCTT
TGAGCTAAGGCGAGGCAACGCCGTACTGGTTTTTGTTAATCCACTATATTTTGCCGTTT
TGAGGCCGGGGTCGGAATAACCGTTTTTTGATGATTTTCCCTCCCCGGCTGTGTCATCA
AAACCCCAATTGCCTTTCCAAACTCTCCACCAGATTGTCATCCAGTTCCAAAGCCTGCG
ACAGGCGGGCGAGGAAGACGGTTTCTTTCGG

TABLE 6C

L3 lot consensus sequence; ATG translation start
codon and TAG translation termination codons
underlined.

(SEQ ID NO:19)

CGCGAGTGGCAGCTGAGCGTTGGCGAACGGATTTGAGCGTTTACTGAAAC
CGATGCCGTCTGAACGCGCGTTCAGACGGCATTTTTAAGATAACGGGACA
TACGGGGGCGATATTTATGCAAGCTGTCCGATACAGGCCTGAAATTGACG
GATTGCGGGCCGTTGCCGTGCTATCCGTCATTATTTTCCACCTGAATAAC
CGCTGGCTGCCCGGAGGGTTTTTGGGGGTGGACATTTTCTTTGTCATCTC
GGGATTCCTCATTACCGGCATCATTCTTTCTGAAATACAGAACGGTTCTT
TTTCTTTCCGGGATTTTTATACCCGCAGGATTAAGCGGATTTATCCTGCT
TTTATCGCGGCCGTGTCGCTGGCTTCGGTGATTGCCTCTCAAATCTTCCT
TTACGAAGATTTCAACCAAATGCGGAAAACCGTGGAGCTTTCTGCGGTTT
TCTTGTCCAATATTTATCTGGGGTTTCAGCAGGGGTATTTCGATTTGAGT
GCCGACGAGAACCCCGTACTGCATATCTGGTCTTTGGCGGTAGAGGAACA
GTATTACCTCCTGTATCCTCTTTTGCTGATATTTTGCTGCAAAAAAACCA
AATCGCTACGGGTGCTGCGTAACATCAGCATCATCCTATTTCTGATTTTG
ACTGCCACATCGTTTTTGCCAAGCAGGTTTTATACCGACATCCTCAACCA
ACCCAATACTTATTACCTTTCGACACTGAGGTTTCCCGAGCTGTTGGCAG
GTTCGCTGCTGGCGGTTTACGGGCAAACGCAAAACGGCAGACGGCAAACA
GCAAGCGGAAAACGGCAGTTGCTTTCATTACTCTGCTTCGGCGCATTGCT
TGCCTGCCTGTTCGTGATTGACAAACACAATCCGTTTATCCCGGGAATGA
CCCTGCTCCTTCCCTGCCTGCTGACGGCACTGCTTATCCGGAGTATGCAA
TACGGGACACTTCCGACCCGCATCCTGTCGGCAAGCCCCATCGTATTTGT
CGGCAAAATCTCTTATTCCCTATACCTGTACCATTGGATTTTTATTGCCT
TCGCCCATTACATTACAGGCGACAAACAGCTCGGACTGCCTGCCGTATCG
GCGGTTGCCGCATTGACGGCCGGATTTTCCCTGTTGAGCTATTATTTGAT
TGAACAGCCGCTTAGAAAACGGAAGATGACCTTCAAAAAGGCATTTTTCT
GCCTCTATCTCGCCCCGTCCCTGATACTTGTCGGTTACAACCTGTACGCA
AGGGGGATATTGAAACAGGAACACCTCCGCCCGTTGCCCGGCGCGCCCCT
TGCTGCGGAAAATCATTTTCCGGAAACCGTCCTGACCCTCGGCGACTCGC
ACGCCGGACACCTGCGGGGTTTTCTGGATTATGTCGGCAGCCGGGAAGGG
TGGAAAGCCAAAATCCTGTCCCTCGATTCGGAGTGTTTGGTTTGGGTGGA
TACGACACTGGCAGACAACCCGTTATGTCGAAAATACCGGGATGAAGTTG
AAAAAGCCGAAGCCGTTTTCATTGCCCAATTCTATGATTTGAGGATGGGC
GGCCAGCCCGTGCCGAGATTTGAAGCGCAATCCTTCCTAATACCCGGGTT
CCAAGCCCGATTCAGGGAAACCGTCAAAAGGATAGCCGCCGTCAAACCCG
TCTATGTTTTTGCAAACAACACATCAATCAGCCGTTCGCCCCTGAGGGAG
GAAAAATTGAAAAGATTTGCCGCAAACCAATATCTCCGCCCCATTCAGGC
TATGGGCGACATCGGCAAGAGCAATCAGGCGGTCTTTGATTTGGTTAAAG
ATATCCCCAATGTGCATTGGGTGGACGCACAGAAATACCTGCCTAAAAAC
ACGGTCGAAATACACGGACGCTATCTTTACGGCGACCAAGACCACCTGAC
CTATTTCGGTTCTTATTATATGGGGCGGGAATTTCACAAACACGAACGCC
TGCTTAAATCTTCCCGCGGCGGCGCATTGCAGTAGCCTGCCTTCTTGTCG
GATATTGCCTTTGGCAGCCTATGCCGCTGTTTGCCCTTCGGGGCGGCGGC
TTTTATAGTGGATTAACAAAAATCAGGACAAGGCGACGAAGCCGCAGACA
GTACAAATAGTACGGAACCGATTCACTTGGTGCTTCAGCACCTTAGAGAA
TCGTTCTCTTTGAGCTAAGGCGAGGCAACGCCGTACTGGTTTTTGTTAAT
CCACTATATTTTGCCGTTTTGAGGCCGGGGTCGGAATAACCGTTTTTTGA
TGATTTTCCCTCCCTGGCTGTGTCATCAAAACCCCAATTGCCTTTCCAAA
CTCTCCACCAGATTGTCATCCAGTTTCAAAGCCTGCGACAGGCGGGCGAG
GAAGACGGTTTCTTTCGG

TABLE 6D

L4 lot consensus sequence; ATG translation start
codon and TAG translation termination codons
underlined.

(SEQ ID NO:20)

AGCGTTGGCGAAACGGATTTGAGCGTTTACTGAAACCGATGCCGTCTGAA
CGCGCGTTCAGACGGCATTTTTAAGATAACGGGACATACGGGGCGATATT
TATGCAAGCTGTCCGATACAGGCCTGAAATTGACGGATTGCGGGCCGTTG
CCGTGCTATCCGTCATTATTTTCCACCTGAATAACCGCTGGCTGCCCGGA
GGGTTTTTGGGGGTGGACATTTTCTTTGTCATCTCGGGATTCCTCATTAC
CGGCATCATTCTTTCTGAAATACAGAACGGTTCTTTTTCTTTCCGGGATT
TTTATACCCGCAGGATTAAGCGGATTTACCCTGCCTTTATTGCGGCCGTG
TCGCTGGCTTCGGTGATTGCCTCTCAAATCTTCCTTTACGAAGATTTCAA
CCAAATGCGGAAAACCGTGGAGCTTTCTGCGGTTTTCTTGTCCAATATTT
ATCTGGGGTTTCAGCAGGGGTATTTCGATTTGAGTGCCGACGAGAACCCC
GTACTGCATATCTGGTCTTTGGCGGTAGAGGAACAGTATTACCTCCTGTA
TCCCCTTTTGCTGATATTTTGCTGCAAAAAAACCAAATCGCTACGGGTGC
TGCGTAACATCAGCATCATCCTGTTTTTGATTTTGACTGCCTCATCGTTT
TTGCCAAGCGGGTTTTATACCGACATCCTCAACCAACCCAATACTTATTA
CCTTTCGACACTGAGGTTTCCCGAGCTGTTGGCAGGTTCGCTGCTGGCGG
TTTACGGGCAAACGCAAAACGGCAGACGGCAAACAGCAAATGGAAAACGG
CAGTTGCTTTCATCACTCTGCTTCGGCGCATTGCTTGCCTGCCTGTTCGT
GATTGACAAACACAATCCGTTTATCCCGGGAATGACCCTGCTCCTTCCCT
GCCTGCTGACGGCACTGCTTATCCGGAGTATGCAATACGGGACACTTCCG
ACCCGCATCCTGTCGGCAAGCCCCATCGTATTTGTCGGCAAAATCTCTTA
TTCCCTATACCTGTACCATTGGATTTTTATTGCTTTCGCCCATTACATTA
CAGGCGACAAACAGCTCGGACTGCCTGCCGTATCGGCGGTTGCCGCGTTG
ACGGCCGGATTTTCCCTGTTGAGTTATTATTTGATTGAACAGCCGCTTAG
AAAACGGAAGATGACCTTCAAAAAGGCATTTTTCTGCCTCTATCTCGCCC
CGTCCCTGATACTTGTCGGTTACAACCTGTACGCAAGGGGGATATTGAAA
CAGGAACACCTCCGCCCGTTGCCCGGCGCGCCCCTTGCTGCGGAAAATCA
TTTTCCGGAAACCGTCCTGACCCTCGGCGACTCGCACGCCGGACACCTGC
GGGGGTTTCTGGATTATGTCGGCAGCCGGGAAGGGTGGAAAGCCAAAATC
CTGTCCCTCGATTCGGAGTGTTTGGTTTGGGTAGATGAGAAGCTGGCAGA
CAACCCGTTATGTCGAAAATACCGGGATGAAGTTGAAAAAGCCGAAGCCG
TTTTCATTGCCCAATTCTATGATTTGAGGATGGGCGGCCAGCCCGTGCCG
AGATTTGAAGCGCAATCCTTCCTAATACCCGGGTTCCAAGCCCGATTCAG
GGAAACCGTCAAAAGGATAGCCGCCGTCAAACCCGTCTATGTTTTTGCAA
ACAACACATCAATCAGCCGTTCGCCCCTGAGGGAGGAAAAATTGAAAAGA
TTTGCCGCAAACCAATATCTCCGCCCCATTCAGGCTATGGGCGACATCGG
CAAGAGCAATCAGGCGGTCTTTGATTTGGTTAAAGATATCCCCAATGTGC
ATTGGGTGGACGCACAGAAATACCTGCCTAAAAACACGGTCGAAATACAC
GGCTGCTATCTTTACGGCGACCAAGACCACCTGACCTATTTCGGTTCTTA
TTATATGGGGCGGGAATTTCACAAACACGAACGCCTGCTTAAATCTTCTC
GCGACGGCGCATTGCAGTAGCCTGCCTTGCCGTCCGATATCGTTTGTGCC
GCCGTTTGCCTTTCGGGGCGGCGGCTTTTATAGTGGATTAACAAAAATCA
GGACAAGGCAACGAAGCCGCAGACAGTACAAATAGTACGGAACCGATTCA
CTTGGTGCTTCAGCACCTTAGAGAATCGTTCTCTTTGAGCTAAGGCGAGG
CAACGCCGTACTGGTTTTTGTTAATCCACTATATTTTGCCGTTTTGAGGC
CGGGGTCGGAATAACCGTTTTTTGATGATTTTCCCTCCCCGGCTGTGTCA
TCAAAACCCCAATTGCCTTTCCAAACTCTCCACCAGATTGTCATCCAGTT
CCAAAGCCTGCGACAGGCGGGCGAGGAAGACGGTTTCTTTCGGGGA

TABLE 6E

L5 lot consensus sequence; ATG translation start
codon and TAG translation termination codons
underlined.

(SEQ ID NO:21)

ACGCGAGTGGGCAGGCTGAGCGTTGGCGAACGGATTTGAGCGTTTACTGA
AACCGATGCCGTCTGAACGCGCGTTCAGACGGCATTTTTAAGATAACGGG
ACATACGGGGGCGATATTTATGCAAGCTGTCCGATACAGGCCTGAAATTG
ACGGATTGCGGGCCGTCGCCGTGCTATCCGTCATGATTTTCCACCTGAAT
GACCGCTGGCTGCCCGGAGGATTCCTGGGGGTGGACATTTTCTTTGTCAT
CTCAGGATTCCTCATTACCGGCATCATTCTTTCTGAAATACAGAACGGTT
CTTTTTCTTTCCGGGATTTTTATACCCGCAGGATTAAGCGGATTTATCCT
GCTTTTATTGCGGCCGTGTCGCTGGCTTCGGTGATTGCCTCTCAAATCTT
CCTTTACGAAGATTTCAACCAAATGCGGAAAACCGTGGAGCTTTCTGCGG
TTTTCTTGTCCAATATTTATCTGGGGTTTCAGCAGGGGTATTTCGATTTG
AGTGCCGACGAGAACCCCGTACTGCATATCTGGTCTTTGGCAGTAGAGGA
ACAGTATTACCTCCTGTATCCTCTTTTGCTGATATTTTGCTGCAAAAAAA
CAAAATCGCTACGGGTGCTGCGTAACATCAGCATCATCCTATTTCTGATT
TTGACTGCCACATCGTTTTTGCCAAGCGGGTTTTATACCGATATTCTCAA
CCAACCCAATACTTATTACCTTTCGACACTGAGGTTTCCCGAGCTGTTGG
CAGGTTCGCTGCTGGCGGTTTACGGGCAAACGCAAAACGGCAGACGGCAA
ACAGCAAATGGAAAACGGCAGTTGCTTTCATCACTCTGCTTCGGCGCATT
GCTTGCCTGCCTGTTCGTGATTGACAAACACAATCCGTTTATCCCGGGAA
TGACCCTGCTCCTTCCCTGCCTGCTGACGGCACTGCTTATCCGGAGTATG
CAATACGGGACACTTCCGACCCGCATCCTGTCGGCAAGCCCCATCGTATT
TGTCGGCAAAATCTCTTATTCCCTATACCTGTACCATTGGATTTTTATTG
CCTTCGCCCATTACATTACAGGCGACAAACAGCTCGGACTGCCTGCCGTA
TCGGCGGTTGCCGCGTTGACGGCCGGATTTTCCCTGTTGAGTTATTATTT
GATTGAACAGCCGCTTAGAAAACGGAAGATGACCTTCAAAAAGGCATTTT
TCTGCCTCTATCTCGCCCCGTCCCTGATACTTGTCGGTTACAACCTGTAC
GCAAGGGGGATATTGAAACAGGAACACCTCCGCCCGTTGCCCGGCGCGCC
CCTTGCTGCAGAAAATCATTTTCCGGAAACCGTCCTGACCCTCGGCGACT
CGCACGCCGGACACCTGCGGGGTTTTCTGGATTATGTCGGCAGCCGGGAA
GGGTGGAAAGCCAAAATCCTGTCCCTCGATTCGGAGTGTTTGGTTTGGGT
AGATGAGAAGCTGGCAGACAACCCGTTATGTCGAAAATACCGGGATGAAG
TTGAAAAAGCCGAAGCCGTTTTCATTGCCCAATTCTATGATTTGAGGATG
GGCGGCCAGCCCGTGCCGAGATTTGAAGCGCAATCCTTCCTAATACCCGG
GTTCCCAGCCCGATTCAGGGAAACCGTCAAAAGGATAGCCGCCGTCAAAC
CCGTCTATGTTTTTGCAAACAACACATCAATCAGCCGTTCGCCCCTGAGG
GAGGAAAAATTGAAAAGATTTGCCGCAAACCAATATCTCCGCCCCATTCA
GGCTATGGGCGACATCGGCAAGAGCAATCAGGCGGTCTTTGATTTGATTA
AAGATATTCCCAATGTGCATTGGGTGGACGCACAAAAATACCTGCCTAAA
AACACGGTCGAAATACACGGCCGCTATCTTTACGGCGACCAAGACCACCT
GACCTATTTCGGTTCTTATTATATGGGGCGGGAATTTCACAAACACGAAC
GCCTGCTTAAATCTTCCCGCGGCGGCGCATTGCAGTAGCCTGCCTTCTTG
TCGGATATTGCCTTTGGCAGCCTATGCCGCTGTTTGCCGTTTTGAGGCCG
GGGTCGGAATAACCGTTTTTTGATGATTTTCCCTCCCCGGCTGTGTCATC
AAAACCCCAATTGCCTTTCCAAACTCTCCACCAGATTGTCATCCAGTTTC
AAAGCCTGCGACAGGCGGGCGAGGAAGACGGTTTCTTTCCGCGAACAAAT
CGA

TABLE 6F

L6 Lot consensus sequence; ATG translation start
codon and TAG translation termination codons
underlined.

(SEQ ID NO:22)

GGGCAGGCTGAGCGTTGGCGAAACGGATTTGAGCGTTTACTGAAACCGAT
GCCGTCTGAACGCGCGTTCAGACGGCATTTTTAAGATAACGGGACATACG
GGGCGATATTTATGCAAGCTGTCCGATACAGGCCTGAAATTGACGGATTG
CGGGCCGTTGCCGTGCTATCCGTCATTATTTTCCACCTGAATAACCGCTG
GCTGCCCGGAGGGTTTTTGGGGGTGGACATTTTCTTTGTCATCTCGGGAT
TCCTCATTACCGGCATCATTCTTTCTGAAATACAGAACGGTTCTTTTTCT
TTCCGGGATTTTTATACCCGCAGGATTAAGCGGATTTACCCTGCCTTTAT
TGCGGCCGTGTCGCTGGCTTCGGTGATTGCCTCTCAAATCTTCCTTTACG
AAGATTTCAACCAAATGCGGAAAACCGTGGAGCTTTCTGCGGTTTTCTTG
TCCAATATTTATCTGGGGTTTCAGCAGGGGTATTTCGATTTGAGTGCCGA
CGAGAACCCCGTACTGCATATCTGGTCTTTGGCGGTAGAGGAACAGTATT
ACCTCCTGTATCCCCTTTTGCTGATATTTTGCTGCAAAAAAACCAAATCG
CTACGGGTGCTGCGTAACATCAGCATCATCCTGTTTTTGATTTTGACTGC
CTCATCGTTTTTGCCAAGCGGGTTTTATACCGACATCCTCAACCAACCCA
ATACTTATTACCTTTCGACACTGAGGTTTCCCGAGCTGTTGGCAGGTTCG
CTGCTGGCGGTTTACGGGCAAACGCAAAACGGCAGACGGCAAACAGCAAA
TGGAAAACGGCAGTTGCTTTCATCACTCTGCTTCGGCGCATTGCTTGCCT
GCCTGTTCGTGATTGACAAACACAATCCGTTTATCCCGGGAATGACCCTG
CTCCTTCCCTGCCTGCTGACGGCACTGCTTATCCGGAGTATGCAATACGG
GACACTTCCGACCCGCATCCTGTCGGCAAGCCCCATCGTATTTGTCGGCA
AAATCTCTTATTCCCTATACCTGTACCATTGGATTTTTATTGCTTTCGCC
CATTACATTACAGGCGACAAACAGCTCGGACTGCCTGCCGTATCGGCGGT
TGCCGCGTTGACGGCCGGATTTTCCCTGTTGAGTTATTATTTGATTGAAC
AGCCGCTTAGAAAACGGAAGATGACCTTCAAAAAGGCATTTTTCTGCCTC
TATCTCGCCCCGTCCCTGATACTTGTCGGTTACAACCTGTACGCAAGGGG
GATATTGAAACAGGAACACCTCCGCCCGTTGCCCGGCGCGCCCCTTGCTG
CGGAAAATCATTTTCCGGAAACCGTCCTGACCCTCGGCGACTCGCACGCC
GGACACCTGCGGGGGTTTCTGGATTATGTCGGCAGCCGGGAAGGGTGGAA
AGCCAAAATCCTGTCCCTCGATTCGGAGTGTTTGGTTTGGGTAGATGAGA
AGCTGGCAGACAACCCGTTATGTCGAAAATACCGGGATGAAGTTGAAAAA
GCCGAAGCCGTTTTCATTGCCCAATTCTATGATTTGAGGATGGGCGGCCA
GCCCGTGCCGAGATTTGAAGCGCAATCCTTCCTAATACCCGGGTTCCAAG
CCCGATTCAGGGAAACCGTCAAAAGGATAGCCGCCGTCAAACCCGTCTAT
GTTTTTGCAAACAACACATCAATCAGCCGTTCGCCCCTGAGGGAGGAAAA
ATTGAAAAGATTTGCCGCAAACCAATATCTCCGCCCCATTCAGGCTATGG
GCGACATCGGCAAGAGCAATCAGGCGGTCTTTGATTTGGTTAAAGATATC
CCCAATGTGCATTGGGTGGACGCACAGAAATACCTGCCTAAAAACACGGT
CGAAATACACGGCTGCTATCTTTACGGCGACCAAGACCACCTGACCTATT
TCGGTTCTTATTATATGGGGCGGGAATTTCACAAACACGAACGCCTGCTT
AAATCTTCTCGCGACGGCGCATTGCAGTAGCCTGCCTTGCCGTCCGATAT
CGTTTGTGCCGCCGTTTGCCTTTCGGGGCGGCGGCTTTTATAGTGGATTA
ACAAAAATCAGGACAAGGCAACGAAGCCGCAGACAGTACAAATAGTACGG
AACCGATTCACTTGGTGCTTCAGCACCTTAGAGAATCGTTCTCTTTGAGC
TAAGGCGAGGCAACGCCGTACTGGTTTTTGTTAATCCACTATATTTTGCC
GTTTTGAGGCCGGGGTCGGAATAACCGTTTTTTGATGATTTTCCCTCCCC
GGCTGTGTCATCAAAACCCCAATTGCCTTTCCAAACTCTCCACCAGATTG
TCATCCAGTTCCAAAGCCTGCGACAGGCGGGCGAGGAAGACGGTTTCTTTC
GGG

TABLE 6G

L7 lot consensus sequence ATG translation start
codon and TAG translation termination codons
underlined.

(SEQ ID NO:23)

AGGGGCAGCTGAGCGTTGGCGAACGGATTTGAGCGTTTACTGAAACCGAT
GCCGTCTGGACGCGCGTTCAGACGGCATTTTTAAAATACCGGATATACAG
GGGCGATATTTATGCAAACTGTCCGATACAGGCCTGAAATTGACGGATTA
CGGGCTGTCGCCGTCCTTTCCGTCATTATTTTCCACCTGAATAACCGTTG
GCTGCCCGGAGGATTCCTGGGGGTGGACATTTTCTTTGTCATCTCGGGAT
TCCTCATTACCGGCATCATTCTTTCTGAAATACAGAACGGTTCTTTTTCT
TTCCGGGATTTTTATACCCGCAGGATTAAGCGGATTTACCCTGCCTTTAT
TGCGGCCGTGTCGCTGGCTTCGGTGATTGCCTCTCAAATCTTCCTTTACG
AAGATTTCAACCAAATGCGGAAAACCGTGGAGCTTTCTGCGGTTTTCTTG
TCCAATATTTATCTGGGGTTTCAGCAGGGGTATTTCGATTTGAGTGCCGA
CGAGAACCCCGTACTGCATATCTGGTCTTTGGCGGTAGAGGAACAGTATT
ACCTCCTGTATCCTCTTTTGCTGATATTTTGCTGCAAAAAAACAAAATCG
CTACGGGTGTTGTGCCACATCAGCATCATCCTGTTTTTGATTTTGACTGC
CTCATCGTTTTTGCCAAGCGGGTTTTATACCGACATCCTCAACCAACCCA
ATACTTATTACCTTTCGACACTGAGGTTTCCCGAGCTGTTGGCAGGTTCG
CTGCTGGCGGTTTACGGGCAAACGCAAAACGGCAGACGGCAAACAGCAAA
TGGAAAACGGCAGTTGCTTTCATCACTCTGCTTCGGCGCATTGCTTGCCT
GCCTGTTCGTGATTGACAAACACAATCCGTTTATCCCGGGAATGACCCTG
CTCCTTCCCTGCCTGCTGACGGCGCTGCTTATCCGGAGTATGCAATACGG
GACACTTCCGACCCGCATCCTGTCGGCAAGCCCCATCGTATTTGTCGGCA
AAATCTCTTATTCCCTATACCTGTACCATTGGATTTTTATTGCCTTCGCC
CATTACATTACAGGCGACAAACAGCTCGGACTGCCTGCCGTATCGGCGGT
TGCCGCGTTGACGGCCGGATTTTCCCTGTTGAGTTATTATTTGATTGAAC
AGCCGCTTAGAAAACGGAAGATGACCTTCAAAAAGGCATTTTTCTGCCTC
TATCTCGCCCCGTCCCTGATACTTGTCGGTTACAACCTGTACGCAAGGGG
GATATTGAAACAGGAACACCTCCGCCCGTTGCCCGGCGCGCCCCTTGCTG
CGGAAAATCATTTTCCGGAAACCGTCCTGACCCTCGGCGACTCGCACGCC
GGACACCTGCGGGGGTTTCTGGATTATGTCGGCAGCCGGGAAGGGTGGAA
AGCCAAAATCCTGTCCCTCGATTCGGAGTGTTTGGTTTGGGTAGATGAGA
AGCTGGCAGACAACCCGTTATGTCGAAAATACCGGGATGAAGTTGAAAAA
GCCGAAGCCGTTTTCATTGCCCAATTCTATGATTTGAGGATGGGCGGCCA
GCCCGTGCCGAGATTTGAAGCGCAATCCTTCCTAATACCCGGGTTCCCAG
CCCGATTCAGGGAAACCGTCAAAAGGATAGCCGCCGTCAAACCCGTCTAT
GTTTTTGCAAACAACACATCAATCAGCCGTTCGCCCCTGAGGGAGGAAAA
ATTGAAAAGATTTGCCGCAAACCAATATCTCCGCCCCATTCAGGCTATGG
GCGACATCGGCAAGAGCAATCAGGCGGTCTTTGATTTGATTAAAGATATT
CCCAATGTGCATTGGGTGGACGCACAAAAATACCTGCCCAAAAACACGGT
CGAAATATACGGCCGCTATCTTTACGGCGACCAAGACCACCTGACCTATT
TCGGTTCTTATTATATGGGGCGGGAATTTCACAAACATGAAAGCTTGCTC
AAGCATTCACACGGCAACGCATTGCAGTAGCCTGCCTTCTTGTCGGATAT
TGCCTTTGGCAGCCTATGCCGCTGTTTGCCCTTCGGGGCGGCGGCTTTTA
TAGTGGATTAACAAAAATCAGGACAAGGCGACGAAGCCGCAGACAGTACA
AATAGTACGGAACCGATTCACTTGGTGCTTCAGCACCTTAGAGAATCGTT
CTCTTTGAGCTAAGGCGAGGCAACGCCGTACTGGTTTTTGTTAATCCACT
ATATTTTGCCGTTTTGAGGCCGGGGTCGGAATAACCGTTTTTTGATGATT
TTCCCTCCCCGGCTGTGTCATCAAAACCCCAATTGCCTTTCCAAACTCTC
CACCAGATTGTCATCCAGTTTCAAAGCCTGCGACAGGCGGGCGAGGAAGA
CGGTTTCTTTCGG

TABLE 6H

L8 lot consensus sequence ATG Translation start
codon and TAG translation termination codons
underlined.

(SEQ ID NO:24)

ATGCAAGCTGTCCGATACAGGCCTGAAATTGACGGATTGCGGGCCGTTGC
CGTGCTATCCGTCATTATTTTCCACCTGAATAACCGCTGGCTGCCCGGAG
GGTTTTTGGGGGTGGACATTTTCTTTGTCATCTCGGGATTCCTCATTACC
GGCATCATTCTTTCTGAAATACAGAACGGTTCTTTTTCTTTCCGGGATTT
TTATACCCGCAGGATTAAGCGGATTTACCCTGCCTTTATTGCGGCCGTGT
CGCTGGCTTCGGTGATTGCCTCTCAAATCTTCCTTTACGAAGATTTCAAC
CAAATGCGGAAAACCGGGGAGTTTTCTGCGGTTTTCTTGTCCAATATTTA
TCTGGGGTTTCAGCAGGGGTATTTCGATTTGAGTGCCGACGAGAACCCCG
TACTGCATATCTGGTCTTTGGCAGTAGAGGAACAGTATTACCTCCTGTAT
CCTCTTTTGCTGATATTTTGCTGCAAAAAAACAAAATCGCTACGGGTGCT
GCGTAACATCAGCATCATCCTATTTCTGATTTTGACTGCCACATCGTTTT
TGCCAAGCGGGTTTTATACCGATATTCTCAACCAACCCAATACTTATTAC
CTTTCGACACTGAGGTTTCCCGAGCTGTTGGCAGGTTCGCTGCTGGCGGT
TTACGGGCAAACGCAAAACGGCAGACGGCAAACAGCAAATGGAAAACGGC
AGTTGCTTTCATCACTCTGCTTCGGCGCATTGCTTGCCTGCCTGTTCGTG
ATTGACAAACACAATCCGTTTATCCCGGGAATGACCCTGCTCCTTCCCTG
CCTGCTGACGGCGCTGCTTATCCGGAGTATGCAATACGGGACACTTCCGA
CCCGCATCCTGTCGGCAAGCTCCATCGTATTTGTCGGCAAAATCTCTTAT
TCCCTATACCTGTACCATTGGATTTTTATTGCTTTCGCCCATTACATTAC
AGGCGACAAACAGCTCGGACTGCCTGCCGTATCGGCGGTTGCCGCGTTGA
CGGCCGGATTTTCCCTGTTGAGTTATTATTTGATTGAACAGCCGCTTAGA
AAACGGAAGATGACCTTCAAAAAGGCATTTTTCTGCCTCTATCTCGCCCC
GTCCCTGATACTTGTCGGTTACAACCTGTACGCAAGGGGGATATTGAAAC
AGGAACACCTCCGCCCGTTGCCCGGCGCGCCCCTTGCTGCGGAAAATCAT
TTTCCGGAAACCGTCCTGACCCTCGGCGACTCGCACGCCGGACACCTGCG
GGGGTTTCTGGATTATGTCGGCAGCCGGGAAGGGTGGAAAGCCAAAATCC
TGTCCCTCGATTCGGAGTGTTTGGTTTGGGTAGATGAGAAGCTGGCAGAC
AACCCGTTATGTCGAAAATACCGGGATGAAGTTGAAAAAGCCGAAGCCGT
TTTCATTGCCCAATTCTATGATTTGAGGATGGGCGGCCAGCCCGTGCCGA
GATTTGAAGCGCAATCCTTCCTAATACCCGGGTTCCAAGCCCGATTCAGG
GAAACCGTCAAAAGGATAGCCGCCGTCAAACCCGTCTATGTTTTTGCAAA
CAACACATCAATCAGCCGTTCGCCCCTGAGGGAGGAAAAATTGAAAAGAT
TTGCCGCAAACCAATATCTCCGCCCCATTCAGGCTATGGGCGACATCGGC
AAGAGCAATCAGGCGGTCTTTGATTTGATTAAAGATATTCCCAATGTGCA
TTGGGTGGACGCACAAAAATACCTGCCCAAAAACACGGTCGAAATATACG
GCCGCTATCTTTACGGCGACCAAGACCACCTGACCTATTTCGGTTCTTAT
TATATGGGGCGGGAATTTCACAAACACGAACGCCTGCTTAAATCTTCTCG
CGACGGCGCATTGCAGTAG

TABLE 6I

L9 Lot consensus

(SEQ ID NO:25)

ATGCAAGCTGTCCGATACAGACCGGAAATTGACGGATTGCGGGCCGTCGC
CGTGCTATCCGTCATGATTTTCCACCTGAATAACCGCTGGCTGCCCGGAG
GATTCCTGGGGGTGGACATTTTCTTTGTCATCTCAGGATTCCTCATTACC
GGCATCATTCTTTCTGAAATACAGAACGGTTCTTTTTCTTTCCGGGATTT
TTATACCCGCAGGATTAAGCGGATTTATCCTGCTTTTATTGCGGCCGTGT
CGCTGGCTTCGGTGATTGCCTCTCAAATCTTCCTTTACGAAGATTTCAAC
CAAATGCGGAAAACCGTGGAGCTTTCTGCGGTTTTCTTGTCCAATATTTA
TCTGGGGTTTCAGCAGGGGTATTTCGATTTGAGTGCCGACGAGAACCCCG
TACTGCATATCTGGTCTTTGGCAGTAGAGGAACAGTATTACCTCCTGTAT
CCTCTTTTGCTGATATTTTGCTGCAAAAAAACAAAATCGCTACGGGTGCT
GCGTAACATCAGCATCATCCTATTTCTGATTTTGACTGCCACATCGTTTT
TGCCAAGCGGGTTTTATACCGATATTCTCAACCAACCCAATACTTATTAC
CTTTCGACACTGAGGTTTCCCGAGCTGTTGGCAGGTTCGCTGCTGGCGGT
TTACGGGCAAACGCAAAACGGCAGACGGCAAACAGCAAATGGAAAACGGC
AGTTGCTTTCATCACTCTGCTTCGGCGCATTGCTTGCCTGCCTGTTCGTG
ATTGACAAACACAATCCGTTTATCCCGGGAATGACCCTGCTCCTTCCCTG
CCTGCTGACGGCACTGCTTATCCGGAGTATGCAATACGGGACACTTCCGA
CCCGCATCCTGTCGGCAAGCCCCATCGTATTTGTCGGCAAAATCTCTTAT
TCCCTATACCTGTACCATTGGATTTTTATTGCTTTCGCCCATTACATTAC
AGGCGACAAACAGCTCGGACTGCCTGCCGTATCGGCGGTTGCCGCGTTGA
CGGCCGGATTTTCCCTGTTGAGTTATTATTTGATTGAACAGCCGCTTAGA
AAACGGAAGATGACCTTCAAAAAGGCATTTTTCTGCCTCTATCTCGCCCC
GTCCCTGATACTTGTCGGTTACAACCTGTACGCAAGGGGGATATTGAAAC
AGGAACACCTCCGCCCGTTGCCCGGCGCGCCCCTTGCTGCGGAAAATCAT
TTTCCGGAAACCGTCCTGACCCTCGGCGACTCGCACGCCGGACACCTGCG
GGGGTTTCTGGATTATGTCGGCAGCCGGGAAGGGTGGAAAGCCAAAATCC
TGTCCCTCGATTCGGAGTGTTTGGTTTGGGTAGATGAGAAGCTGGCAGAC
AACCCGTTATGTCGAAAATACCGGGATGAAGTTGAAAAAGCCGAAGCCGT
TTTCATTGCCCAATTCTATGATTTGAGGATGGGCGGCCAGCCCGTGCCGA
GATTTGAAGCGCAATCCTTCCTAATACCCGGGTTCCCAGCCCGATTCAGG
GAAACCGTCAAAAGGATAGCCGCCGTCAAACCCGTCTATGTTTTTGCAAA
CAACACATCAATCAGCCGTTCGCCCCTGAGGGAGGAAAAATTGAAAAGAT
TTGCCGCAAACCAATATCTCCGCCCCATTCAGGCTATGGGCGACATCGGC
AAGAGCAATCAGGCGGTCTTTGATTTGATTAAAGATATTCCCAATGTGCA
TTGGGTGGACGCACAAAAATACCTGCCCAAAAACACGGTCGAAATATACG
GCCGCTATCTTTACGGCGACCAAGACCACCTGACCTATTTCGGTTCTTAT
TATATGGGGCGGGAATTTCACAAACACGAACGCCTGCTTAAATCTTCTCG
CGACGGCGCATTGCAGTAG

TABLE 6J

>L10 lot consensus sequence

(SEQ ID NO:26)

GGGCAGGCTGAGCGTTGGCGAAACGGATTTGAGCGTTTACTGAAACCGAT
GCCGTCTGAACGCGCGTTCAGACGGCATTTTTAAGATAACGGGACATACA
GGGGCGATATTTATGCAAGCTGTCCGATACAGACCGGAAATTGACGGATT
GCGGGCCGTCGCCGTGCTATCCGTCATGATTTTCCACCTGAATAACCGCT
GGCTGCCCGGAGGATTCCTGGGGGTGGACATTTTCTTTGTCATCTCAGGA
TTCCTCATTACCGGCATCATTCTTTCTGAAATACAGAACGGTTCTTTTTC
TTTCCGGGATTTTTATACCCGCAGGATTAAGCGGATTTATCCTGCTTTTA
TTGCGGCCGTGTCGCTGGCTTCGGTGATTGCCTCTCAAATCTTCCTTTAC
GAAGATTTCAACCAAATGCGGAAAACCGTGGAGCTTTCTGCGGTTTTCTT
GTCCAATATTTATCTGGGGTTTCAGCAGGGGTATTTCGATTTGAGTGCCG
ACGAGAACCCCGTACTGCATATCTGGTCTTTGGCAGTAGAGGAACAGTAT
TACCTCCTGTATCCTCTTTTGCTGATATTTTGCTGCAAAAAAACAAAATC
GCTACGGGTGCTGCGTAACATCAGCATCATCCTATTTCTGATTTTGACTG
CCACATCGTTTTTGCCAAGCGGGTTTTATACCGATATTCTCAACCAACCC
AATACTTATTACCTTTCGACACTGAGGTTTCCCGAGCTGTTGGCAGGTTC
GCTGCTGGCGGTTTACGGGCAAACGCAAAACGGCAGACGGCAAACAGCAA
ATGGAAAACGGCAGTTGCTTTCATCACTCTGCTTCGGCGCATTGCTTGCC
TGCCTGTTCGTGATTGACAAACACAATCCGTTTATCCCGGGAATGACCCT
GCTCCTTCCCTGCCTGCTGACGGCACTGCTTATCCGGAGTATGCAATACG
GGACACTTCCGACCCGCATCCTGTCGGCAAGCCCCATCGTATTTGTCGGC
AAAATCTCTTATTCCCTATACCTGTACCATTGGATTTTTATTGCTTTCGC
CCATTACATTACAGGCGACAAACAGCTCGGACTGCCTGCCGTATCGGCGG
TTGCCGCGTTGACGGCCGGATTTTCCCTGTTGAGTTATTATTTGATTGAA
CAGCCGCTTAGAAAACGGAAGATGACCTTCAAAAAGGCATTTTTCTGCCT
CTATCTCGCCCCGTCCCTGATACTTGTCGGTTACAACCTGTACGCAAGGG
GGATATTGAAACAGGAACACCTCCGCCCGTTGCCCGGCGCGCCCCTTGCT
GCAGAAAATCATTTTCCGGAAACCGTCCTGACCCTCGGCGACTCGCACGC
CGGACACCTGCGGGGTTTTCTGGATTATGTCGGCAGCCGGGAAGGGTGGA
AAGCCAAAATCCTGTCCCTCGATTCGGAGTGTTTGGTTTGGGTAGATGAG
AAGCTGGCAGACAACCCGTTATGTCGAAAATACCGGGATGAAGTTGAAAA
AGCCGAAGCCGTTTTCATTGCCCAATTCTATGATTTGAGGATGGGCGGCC
AGCCCGTGCCGAGATTTGAAGCGCAATCCTTCCTAATACCCGGGTTCCCA
GCCCGATTCAGGGAAACCGTCAAAAGGATAGCCGCCGTCAAACCCGTCTA
TGTTTTTGCAAACAACACATCAATCAGCCGTTCGCCCCTGAGGGAGGAAA
AATTGAAAAGATTTGCCGCAAACCAATATCTCCGCCCCATTCAGGCTATG
GGCGACATCGGCAAGAGCAATCAGGCGGTCTTTGATTTGATTAAAGATAT
TCCCAATGTGCATTGGGTGGACGCACAAAAATACCTGCCCAAAAACACGG
TCGAAATATACGGCCGCTATCTTTACGGCGACCAAGACCACCTGACCTAT
TTCGGTTCTTATTATATGGGGCGGGAATTTCACAAACACGAACGCCTGCT
TAAATCTTCTCGCGACGGCGCATTGCAGTAGCCTGCCTTGCCGTCCGATA
TCGTTTGTGCCGCCGTTTGCCTTTCGGGGCGGCGGCGGTTTTTATTTTCC
TTCCCCTGCGGGAGGGAATTTTGAATCAAAACCCCAATTGCCTTTCCAAG
TTTTCCACCAGATTGTCATCCAGTTCCAAAGCCTGCGACAGGCGGGCGAG
GAAGACGGTTTCTTTCCCGAACAAATCGAA

TABLE 6K

L11 consensus sequence; ATG translation start
codon and TAG translation termination codons
underlined.

(SEQ ID NO:27)

GTGGGCAGGCTGAGCGTTGGCGAAACGGATTTGAGCGTTTACTGAAACCG
ATGCCGTCTGAACGCGCGTTCAGACGGCATTTTTAAGATAACGGGACATA
CAGGGGCGATATTTATGCAAGCTGTCCGATACAGACCGGAAATTGACGGA
TTGCGGGCCGTCGCCGTGCTATCCGTCATGATTTTCCACCTGAATAACCG
CTGGCTGCCCGGAGGATTCCTGGGGGTGGACATTTTCTTTGTCATCTCAG
GATTCCTCATTACCGGCATCATTCTTTCTGAAATACAGAACGGTTCTTTT
TCTTTCCGGGATTTTTATACCCGCAGGATTAAGCGGATTTATCCTGCTTT
TATTGCGGCCGTGTCGCTGGCTTCGGTGATTGCCTCTCAAATCTTCCTTT
ACGAAGATTTCAACCAAATGCGGAAAACCGTGGAGCTTTCTGCGGTTTTC
TTGTCCAATATTTATCTGGGGTTTCAGCAGGGGTATTTCGATTTGAGTGC
CGACGAGAACCCCGTACTGCATATCTGGTCTTTGGCAGTAGAGGAACAGT
ATTACCTCCTGTATCCTCTTTTGCTGATATTTTGCTGCAAAAAAACAAAA
TCGCTACGGGTGCTGCGTAACATCAGCATCATCCTATTTCTGATTTTGAC
TGCCACATCGTTTTTGCCAAGCGGGTTTTATACCGATATTCTCAACCAAC
CCAATACTTATTACCTTTCGACACTGAGGTTTCCCGAGCTGTTGGCAGGT
TCGCTGCTGGCGGTTTACGGGCAAACGCAAAACGGCAGACGGCAAACAGC
AAATGGAAAACGGCAGTTGCTTTCATCACTCTGCTTCGGCGCATTGCTTG
CCTGCCTGTTCGTGATTGACAAACACAATCCGTTTATCCCGGGAATGACC
CTGCTCCTTCCCTGCCTGCTGACGGCACTGCTTATCCGGAGTATGCAATA
CGGGACACTTCCGACCCGCATCCTGTCGGCAAGCCCCATCGTATTTGTCG
GCAAAATCTCTTATTCCCTATACCTGTACCATTGGATTTTTATTGCTTTC
GCCCATTACATTACAGGCGACAAACAGCTCGGACTGCCTGCCGTATCGGC
GGTTGCCGCGTTGACGGCCGGATTTTCCCTGTTGAGTTATTATTTGATTG
AACAGCCGCTTAGAAAACGGAAGATGACCTTCAAAAAGGCATTTTTCTGC
CTCTATCTCGCCCCGTCCCTGATACTTGTCGGTTACAACCTGTACGCAAG
GGGGATATTGAAACAGGAACACCTCCGCCCGTTGCCCGGCGCGCCCCTTG
CTGCGGAAAATCATTTTCCGGAAACCGTCCTGACCCTCGGCGACTCGCAC
GCCGGACACCTGCGGGGGTTTCTGGATTATGTCGGCAGCCGGGAAGGGTG
GAAAGCCAAAATCCTGTCCCTCGATTCGGAGTGTTTGGTTTGGGTAGATG
AGAAGCTGGCAGACAACCCGTTATGTCGAAAATACCGGGATGAAGTTGAA
AAAGCCGAAGCCGTTTTCATTGCCCAATTCTATGATTTGAGGATGGGCGG
CCAGCCCGTGCCGAGATTTGAAGCGCAATCCTTCCTAATACCCGGGTTCC
CAGCCCGATTCAGGGAAACCGTCAAAAGGATAGCCGCCGTCAAACCCGTC
TATGTTTTTGCAAACAACACATCAATCAGCCGTTCGCCCCTGAGGGAGGA
AAAATTGAAAAGATTTGCCGCAAACCAATATCTCCGCCCCATTCAGGCTA
TGGGCGACATCGGCAAGAGCAATCAGGCGGTCTTTGATTTGATTAAAGAT
ATTCCCAATGTGCATTGGGTGGACGCACAAAAATACCTGCCCAAAAACAC
GGTCGAAATATACGGCCGCTATCTTTACGGCGACCAAGACCACCTGACCT
ATTTCGGTTCTTATTATATGGGGCGGGAATTTCACAAACACGAACGCCTG
CTTAAATCTTCTCGCGACGGCGCATTGCAGTAG CCTGCCTTGCCGTCCGA
TATCGTTTGTGCCGCCGTTTGCCTTTCGGGGCGGCGGCGGTTTTTATTTT
CCTTCCCCTGCGGGAGGGAATTTTGAATCAAAACCCCAATTGCCTTTCCA
AGTTTTCCACCAGATTGTCATCCAGTTCCAAAGCCTGCGACAGGCGGGCG
AGGAAGACGGTTTCTTTCCGCGAACAAATCGAA

TABLE 6L

L12 Lot consensus sequence; ATG translation start
codon and TAG translation termination codons
underlined.

(SEQ ID NO:28)

ATGCAAGCTGTCCGATACAGACCGGAAATTGACGGATTGCGGGCCGTCGC
CGTGCTATCCGTCATGATTTTCCACCTGAATAACCGCTGGCTGCCCGGAG
GATTCCTGGGGGTGGACATTTTCTTTGTCATCTCAGGATTCCTCATTACC
GGCATCATTCTTTCTGAAATACAGAACGGTTCTTTTTCTTTCCGGGATTT
TTATACCCGCAGGATTAAGCGGATTTATCCTGCTTTTATTGCGGCCGTGT
CGCTGGCTTCGGTGATTGCCTCTCAAATCTTCCTTTACGAAGATTTCAAC
CAAATGCGGAAAACCGTGGAGCTTTCTGCGGTTTTCTTGTCCAATATTTA
TCTGGGGTTTCAGCAGGGGTATTTCGATTTGAGTGCCGACGAGAACCCCG
TACTGCATATCTGGTCTTTGGCAGTAGAGGAACAGTATTACCTCCTGTAT
CCTCTTTTGCTGATATTTTGCTGCAAAAAAACAAAATCGCTACGGGTGCT
GCGTAACATCAGCATCATCCTATTTCTGATTTTGACTGCCACATCGTTTT
TGCCAAGCGGGTTTTATACCGATATTCTCAACCAACCCAATACTTATTAC
CTTTCGACACTGAGGTTTCCCGAGCTGTTGGCAGGTTCGCTGCTGGCGGT
TTACGGGCAAACGCAAAACGGCAGACGGCAAACAGCAAATGGAAAACGGC
AGTTGCTTTCATCACTCTGCTTCGGCGCATTGCTTGCCTGCCTGTTCGTG
ATTGACAAACACAATCCGTTTATCCCGGGAATGACCCTGCTCCTTCCCTG
CCTGCTGACGGCACTGCTTATCCGGAGTATGCAATACGGGACACTTCCGA
CCCGCATCCTGTCGGCAAGCCCCATCGTATTTGTCGGCAAAATCTCTTAT
TCCCTATACCTGTACCATTGGATTTTTATTGCTTTCGCCCATTACATTAC
AGGCGACAAACAGCTCGGACTGCCTGCCGTATCGGCGGTTGCCGCGTTGA
CGGCCGGATTTTCCCTGTTGAGTTATTATTTGATTGAACAGCCGCTTAGA
AAACGGAAGATGACCTTCAAAAAGGCATTTTTCTGCCTCTATCTCGCCCC
GTCCCTGATACTTGTCGGTTACAACCTGTACGCAAGGGGGATATTGAAAC
AGGAACACCTCCGCCCGTTGCCCGGCGCGCCCCTTGCTGCGGAAAATCAT
TTTCCGGAAACCGTCCTGACCCTCGGCGACTCGCACGCCGGACACCTGCG
GGGGTTTCTGGATTATGTCGGCAGCCGGGAAGGGTGGAAAGCCAAAATCC
TGTCCCTCGATTCGGAGTGTTTGGTTTGGGTAGATGAGAAGCTGGCAGAC
AACCCGTTATGTCGAAAATACCGGGATGAAGTTGAAAAAGCCGAAGCCGT
TTTCATTGCCCAATTCTATGATTTGAGGATGGGCGGCCAGCCCGTGCCGA
GATTTGAAGCGCAATCCTTCCTAATACCCGGGTTCCCAGCCCGATTCAGG
GAAACCGTCAAAAGGATAGCCGCCGTCAAACCCGTCTATGTTTTTGCAAA
CAACACATCAATCAGCCGTTCGCCCCTGAGGGAGGAAAAATTGAAAAGAT
TTGCCGCAAACCAATATCTCCGCCCCATTCAGGCTATGGGCGACATCGGC
AAGAGCAATCAGGCGGTCTTTGATTTGATTAAAGATATTCCCAATGTGCA
TTGGGTGGACGCACAAAAATACCTGCCCAAAAACACGGTCGAAATATACG
GCCGCTATCTTTACGGCGACCAAGACCACCTGACCTATTTCGGTTCTTAT
TATATGGGGCGGGAATTTCACAAACACGAACGCCTGCTTAAATCTTCTCG
CGACGGCGCATTGCAGTAG

The lot genes are intact in N. meningitidis strain Z2491 and in N. gonorrhoeae strain FA1090. However, in N. gonorrhoeae the lot gene has been duplicated in two separate loci. There is a translational frameshift in N. meningitidis strain MC58.

See additional lot coding sequences below.

TABLE 7

Neisseria meningitidis Strain Z2491

NMA2202 Coding sequence

(SEQ ID NO:29)

ATGCAAGCTGTCCGATACAGACCGGAAATTGACGGATTGCGGGCCGTCGC
CGTGCTATCCGTCATGATTTTCCACCTGAATAACCGCTGGCTGCCCGGAG
GATTCCTGGGGGTGGACATTTTCTTTGTCATCTCAGGATTCCTCATTACC
GGCATCATTCTTTCTGAAATACAGAACGGTTCTTTTTCTTTCCGGGATTT
TTATACCCGCAGGATTAAGCGGATTTATCCTGCTTTTATTGCGGCCGTGT
CGCTGGCTTCGGTGATTGCCTCTCAAATCTTCCTTTACGAAGATTTCAAC
CAAATGCGGAAAACCGTGGAGCTTTCTGCGGTTTTCTTGTCCAATATTTA
TCTGGGGTTTCAGCAGGGGTATTTCGATTTGAGTGCCGACGAGAACCCCG
TACTGCATATCTGGTCTTTGGCAGTAGAGGAACAGTATTACCTCCTGTAT
CCTCTTTTGCTGATATTTTGCTGCAAAAAAACAAAATCGCTACGGGTGCT
GCGTAACATCAGCATCATCCTATTTCTGATTTTGACTGCCACATCGTTTT
TGCCAAGCGGGTTTTATACCGATATTCTCAACCAACCCAATACTTATTAC
CTTTCGACACTGAGGTTTCCCGAGCTGTTGGCAGGTTCGCTGCTGGCGGT
TTACGGGCAAACGCAAAACGGCAGACGGCAAACAGCAAATGGAAAACGGC
AGTTGCTTTCATCACTCTGCTTCGGCGCATTGCTTGCCTGCCTGTTCGTG
ATTGACAAACACAATCCGTTTATCCCGGGAATGACCCTGCTCCTTCCCTG
CCTGCTGACGGCACTGCTTATCCGGAGTATGCAATACGGGACACTTCCGA
CCCGCATCCTGTCGGCAAGCCCCATCGTATTTGTCGGCAAAATCTCTTAT
TCCCTATACCTGTACCATTGGATTTTTATTGCTTTCGCCCATTACATTAC
AGGCGACAAACAGCTCGGACTGCCTGCCGTATCGGCGGTTGCCGCGTTGA
CGGCCGGATTTTCCCTGTTGAGTTATTATTTGATTGAACAGCCGCTTAGA
AAACGGAAGATGACCTTCAAAAAGGCATTTTTCTGCCTCTATCTCGCCCC
GTCCCTGATACTTGTCGGTTACAACCTGTACGCAAGGGGGATATTGAAAC
AGGAACACCTCCGCCCGTTGCCCGGCGCGCCCCTTGCTGCGGAAAATCAT
TTTCCGGAAACCGTCCTGACCCTCGGCGACTCGCACGCCGGACACCTGCG
GGGGTTTCTGGATTATGTCGGCAGCCGGGAAGGGTGGAAAGCCAAAATCC
TGTCCCTCGATTCGGAGTGTTTGGTTTGGGTAGATGAGAAGCTGGCAGAC
AACCCGTTATGTCGAAAATACCGGGATGAAGTTGAAAAAGCCGAAGCCGT
TTTCATTGCCCAATTCTATGATTTGAGGATGGGCGGCCAGCCCGTGCCGA
GATTTGAAGCGCAATCCTTCCTAATACCCGGGTTCCCAGCCCGATTCAGG
GAAACCGTCAAAAGGATAGCCGCCGTCAAACCCGTCTATGTTTTTGCAAA
CAACACATCAATCAGCCGTTCGCCCCTGAGGGAGGAAAAATTGAAAAGAT
TTGCCGCAAACCAATATCTCCGCCCCATTCAGGCTATGGGCGACATCGGC
AAGAGCAATCAGGCGGTCTTTGATTTGATTAAAGATATTCCCAATGTGCA
TTGGGTGGACGCACAAAAATACCTGCCCAAAAACACGGTCGAAATATACG
GCCGCTATCTTTACGGCGACCAAGACCACCTGACCTATTTCGGTTCTTAT
TATATGGGGCGGGAATTTCACAAACACGAACGCCTGCTTAAATCTTCTCG
CGACGGCGCATTGCAGTAG
NMA2202 Encoded amino acid sequence

(SEQ ID NO:30)

MQAVRYRPEIDGLRAVAVLSVMIFHLNNRWLPGGFLGVDIFFVISGFLIT
GIILSEIQNGSFSFRDFYTRRIKRIYPAFIAAVSLASVIASQIFLYEDFN
QMRKTVELSAVFLSNIYLGFQQGYFDLSADENPVLHIWSLAVEEQYYLLY
PLLLIFCCKKTKSLRVLRNISIILFLILTATSFLPSGFYTDILNQPNTYY
LSTLRFPELLAGSLLAVYGQTQNGRRQTANGKRQLLSSLCFGALLACLFV
IDKHNPFIPGMTLLLPCLLTALLIRSMQYGTLPTRILSASPIVFVGKISY
SLYLYHWIFIAFAHYITGDKQLGLPAVSAVAALTAGFSLLSYYLIEQPLR
KRKMTFKKAFFCLYLAPSLILVGYNLYARGILKQEHLRPLPGAPLAAENH
FPETVLTLGDSHAGHLRGFLDYVGSREGWKAKILSLDSECLVWVDEKLAD
NPLCRKYRDEVEKAEAVFIAQFYDLRMGGQPVPRFEAQSFLIPGFPARFR
ETVKRIAAVKPVYVFANNTSISRSPLREEKLKRFAANQYLRPIQAMGDIG
KSNQAVFDLIKDIPNVHWVDAQKYLPKNTVEIYGRYLYGDQDHLTYFGSY
YMGREFHKHERLLKSSRDGALQ

TABLE 8

Neisseria gonorrhoeae strain FA1090

NGO1710 Coding sequence

(SEQ ID NO:31)

ATGCAAGCTGTCCGATACAGGCCTGAAATTGACGGATTGCGGGCCGTCGC
CGTGCTATCCGTCATTATTTTCCACCTGAATAACCGCTGGCTGCCCGGAG
GATTCCTGGGGGTGGACATTTTCTTTGTCATCTCGGGATTCCTCATTACC
AACATCATTCTTTCTGAAATACAGAACGGTTCTTTTTCTTTCCGGGATTT
TTATACCCGCAGGATTAAGCGGATTTATCCTGCTTTTATTGCGGCCGTGT
CCCTGGCTTCGGTGATTGCTTCTCAAATCTTCCTTTACGAAGATTTCAAC
CAAATGAGGAAAACCATAGAGCTTTCTACGGTTTTTTTGTCCAATATTTA
TTTGGGGTTCCGATTGGGGTATTTCGATTTGAGTGCCGACGAGAACCCCG
TACTGCATATCTGGTCTTTGGCGGTAGAGGAACAGTATTACCTCCTGTAT
CCTCTTTTGCTGATATTCTGTTACAAAAAAACCAAATCACTACGGGTGCT
GCGTAATATCAGCATCATCCTGTTTCTGATTTTGACCGCATCATCGTTTT
TGCCGGCCGGGTTTTATACCGACATCCTCAACCAACCCAATACTTATTAC
CTTTCGACACTGAGGTTTCCCGAGCTGTTGGTGGGTTCGCTGTTGGCGGT
TTACGGGCAAACGCAAAACGGCAGACGGCAAACAGAAAATGGAAAACGGC
AGTTGCTTTCATTACTCTGTTTCGGCGCATTGCTTGTCTGCCTGTTCGTG
ATCGACAAACACGATCCGTTTATCCCGGGAATAACCCTGCTCCTTCCCTG
CCTGCTGACGGCGCTGCTTATCCGGAGTATGCAATACGGGACACTTCCGA
CCCGCATCCTGTCGGCAAGCCCCATCGTATTTGTCGGCAAAATCTCTTAT
TCCCTATACCTGTACCATTGGATTTTTATTGCCTTCGCCCATTACATTAC
AGGCGACAAACAGCTCGGACTGCCTGCCGTATCGGCGGTTGCCGCGTTGA
CGGCCGGATTTTCCCTGTTGAGCTATTATTTGATTGAACAGCCGCTTAGA
AAACGGAAGATGACCTTCAAAAAGGCATTTTTCTGCCTTTATCTCGCCCC
GTCCCTGATGCTTGTCGGTTACAACCTGTATTCAAGAGGGATATTGAAAC
AGGAACACCTCCGCCCGCTGCCCGGCACGCCCGTTGCTGCGGAAAATAAT
TTTCCGGAAACCGTCTTGACCCTCGGCGACTCGCACGCCGGACACCTGCG
GGGGTTTCTGGATTATGTCGGCGGCAGGGAAGGGTGGAAAGCTAAAATCC
TGTCCCTCGATTCGGAGTGTTTGGTTTGGGTGGATGAGAAGCTGGCAGAC
AACCCGTTGTGCCGAAAATACCGGGATGAAGTTGAAAAAGCCGAAGCTGT
TTTCATTGCCCAATTCTATGATTTGAGGATGGGCGGCCAGCCCGTGCCGA
GATTTGAAGCGCAATCCTTCCTGATACCCGGGTTCAAAGCCCGATTCAGG
GAAACCGTCAAGAGGATAGCCGCCGTCAAACCTGTATATGTTTTTGCAAA
CAATACATCAATCAGCCGTTCTCCCTTGAGGGAGGAAAAATTGAAAAGAT
TTGCTATAAACCAATACCTCCGGCCTATTCGGGCTATGGGCGACATCGGC
AAGAGCAATCAGGCGGTCTTTGATTTGGTTAAAGATATTCCCAATGTGCA
TTGGGTGGACGCACAAAAATACCTGCCCAAAAACACGGTCGAAATACACG
GACGCTATCTTTACGGCGACCAAGACCACCTGACCTATTTCGGTTCTTAT
TATATGGGGCGGGAATTTCACAAACACGAACGCCTGCTCAAGCATTCCCG
AGGCGGCGCATTGCAGTAG

TABLE 9

NGO1710 Encoded amino acid sequence

(SEQ ID NO:32)

MQAVRYRPEIDGLRAVAVLSVIIFHLNNRWLPGGFLGVDIFFVISGFLIT
NIILSEIQNGSFSFRDFYTRRIKRIYPAFIAAVSLASVIASQIFLYEDFN
QMRKTIELSTVFLSNIYLGFRLGYFDLSADENPVLHIWSLAVEEQYYLLY
PLLLIFCYKKTKSLRVLRNISIILFLILTASSFLPAGFYTDILNQPNTYY
LSTLRFPELLVGSLLAVYGQTQNGRRQTENGKRQLLSLLCFGALLVCLFV
IDKHDPFIPGITLLLPCLLTALLIRSMQYGTLPTRILSASPIVFVGKISY
SLYLYHWIFIAFAHYITGDKQLGLPAVSAVAALTAGFSLLSYYLIEQPLR
KRKMTFKKAFFCLYLAPSLMLVGYNLYSRGILKQEHLRPLPGTPVAAENN
FPETVLTLGDSHAGHLRGFLDYVGGREGWKAKILSLDSECLVWVDEKLAD
NPLCRKYRDEVEKAEAVFIAQFYDLRMGGQPVPRFEAQSFLIPGFKARFR
ETVKRIAAVKPVYVFANNTSISRSPLREEKLKRFAINQYLRPIRAMGDIG
KSNQAVFDLVKDIPNVHWVDAQKYLPKNTVEIHGRYLYGDQDHLTYFGSY
YMGREFHKHERLLKHSRGGALQ

TABLE 10

NGO0065 Coding sequence

(SEQ ID NO:33)

ATGAGCCAAGCCTTACCCTACCGCCCGGACATCGACACATTGCGCGCCGC
CGCCGTCTTGTCCGTCATCGTGTTCCATATCGAAAAGGATTGGCTGCCGG
GCGGGTTTCTCGGTGTCGATATATTCTTTGTGATTTCAGGCTTTTTGATG
ACGGCGATCCTCCTTCGCGAAATGTCCGGGGGGCGTTTCTTCCTCAAGAC
ATTTTATATCCGCCGCATCAAACGGATTTTGCCCGCATTTTTCGCCGTAT
TGGCGGCAACGCTGGCAGGCGGCTTCTTTTTATTCACCAAAGATGATTTC
TTTCTTTTGTGGAAATCCGCGCTGACCGCCTTGGGTTTCGCCTCCAACCT
GTATTTTGCAAGGGGGAAGGATTATTTCGATCCCGCGCAGGAAGAAAAGC
CCCTGCTGCACATCTGGTCTTTGTCGGTCGAAGAACAATTTTACTTTGTC
TTTCCGATATTGCTGTTGCTTGTCGCCCGCAAAAGCCTGCGCGTACAGTT
CGGCTTCCTCGCCGCATTGTGCGCCTTAAGCCTTGCCGCTTCCTTTATGC
CTTCCGCGCTCGATAAATATTACCTGCCCCACCTGCGCGCCTGCGAAATG
CTGGTCGGATCGCTGACCGCCGTGCGGATGCGGTACCGGCAACAGCGGAA
TCCCGCCGTCGGGAAACGGTATGCCGCCGTCGGCGCATTGTTTTCCGCGT
GCATACTGTCCGCCTGCCTGTTTGCCTATTCGGAACAAACCGCCTATTTC
CCGGGCCCCGCCGCTTTGATTCCCTGTCTGGCTGTTGCCGCGCTGATTTA
TTTCAACCATTACGAACACCCGCTTAAAAAATTTTTCCAATGGAAAATCA
CCGTTGCCGCCGGTTTGATTTCCTATTCGCTTTATCTGTGGCATTGGCCG
ATATTGGCCTTTATGCGCTATATCGGCCCGGACAACCTGCCGCCTTATTC
GCCGGCGGCAGCGATCGTCCTGACCCTGGCGTTTTCCCTGATTTCTTATC
ACTGCATCGAAAAGCCGTTTAAAAAATGGAAAGGCTCGTTCGCACAATCC
GTTTTATGGATTTATGCCTTGCCTATGCTCGTTTTGGGCGCGGGCTCGTT
TTTCGCGATGAGGCTGCCGTTTATGGCGCAATACGACCGCTTGGGGCTGA
CGCGTTCCAACACCTCCTGCCACAACAATACCGGCAAACAATGCCTGTGG
GGGGATACGGAAAAACAGCCGGAACTGCTGGTTTTGGGCGACTCCCACGC
CGACCATTACAAAACATTCTTCGATGCCGTGGGCAAAAAAGAAAAATGGT
CCGCCACTATGGTTTCCGCCGACGCCTGCGCCTATGTGGAAGGCTACGCG
TCCCGTGTGTTCCAAAACTGGGCCGCCTGCCGCGCCGTTTACCGCTATGC
CGAAGAACACCTGCCCCGGTATCCGAAAGTGGTTTTGGCGATGCGCTGGG
GCAGCCAGATGCCCGAAAACAGCCGCTCCCTTGCCTACGATGCCGGTTTT
TTCCAAAAATTCGACCGTATGCTGCACAAACTCTCATCCGAAAAACAAGC
CGTTTACCTGATGGCGGACAACTTGGCTTCGTCTTACAACGTCCAGCGCG
CCTATATCTTGTCTTCACGCATACCGGGTTGCCGCCAAACACTGCGCCCG
GACGACGAAAGCACCCTGAAAGCCAATGCCCGCATCAGGGAATTGGCAGC
CAAATACCCCAACGTCTATATTATTGATGCCGCCGCCTATATCCCCGCAG
ATTTCCAAATCGGCGGATTGCCGGTTTACTCGGACAAAGACCACATCAAC
CCTTACGGCGGCACAGAATTGGCGAAGCGTTTTTCCGAAAAACAAAGGTT
TCTCGATACGCGCCATAACCATTGA

TABLE 11

NGO0065 Encoded amino acid sequence

(SEQ ID NO:34)

MSQALPYRPDIDTLRAAAVLSVIVFHIEKDWLPGGFLGVDIFFVISGFLM
TAILLREMSGGRFFLKTFYIRRIKRILPAFFAVLAATLAGGFFLFTKDDF
FLLWKSALTALGFASNLYFARGKDYFDPAQEEKPLLHIWSLSVEEQFYFV
FPILLLLVARKSLRVQFGFLAALCALSLAASFMPSALDKYYLPHLRACEM
LVGSLTAVRMRYRQQRNPAVGKRYAAVGALFSACILSACLFAYSEQTAYF
PGPAALIPCLAVAALIYFNHYEHPLKKFFQWKITVAAGLISYSLYLWHWP
ILAFMRYIGPDNLPPYSPAAAIVLTLAFSLISYHCIEKPFKKWKGSFAQS
VLWIYALPMLVLGAGSFFAMRLPFMAQYDRLGLTRSNTSCHNNTGKQCLW
GDTEKQPELLVLGDSHADHYKTFFDAVGKKEKWSATMVSADACAYVEGYA
SRVFQNWAACRAVYRYAEEHLPRYPKVVLAMRWGSQMPENSRSLAYDAGF
FQKFDRMLHKLSSEKQAVYLMADNLASSYNVQRAYILSSRIPGCRQTLRP
DDESTLKANARIRELAAKYPNVYIIDAAAYIPADFQIGGLPVYSDKDHIN
PYGGTELAKRFSEKQRFLDTRHNH

This Sanger Center database contains N. meningitidis strain FAM18 (serogroup C) and N. lactamica (commensal neisseria). Neither database is annotated. Nucleotide blast alignments are given below indicating co-ordinates in the respective genomes.

TABLE 12
Neisseria meningitidis serogroup C FAM18
Length = 2,194,961
Plus Strand HSPs:
Score = 9120 (1374.4 bits), Expect = 0., P = 0.
Identities = 1844/1869 (98%), Positives = 1844/1869 (98%), Strand = Plus/Plus

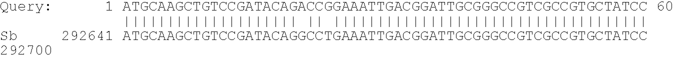

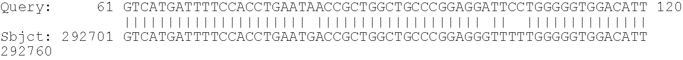

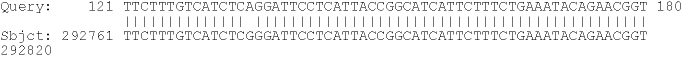

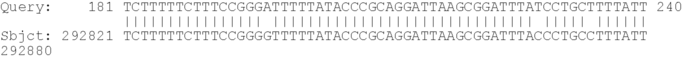

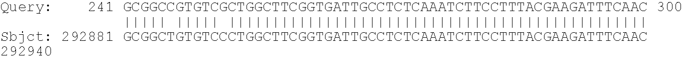

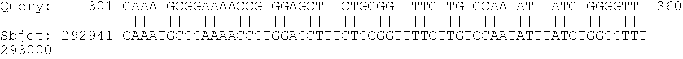

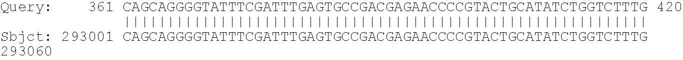

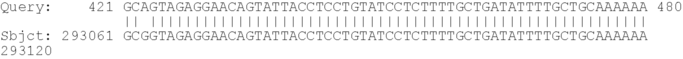

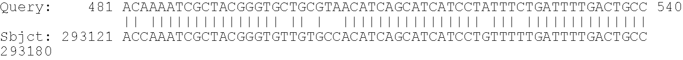

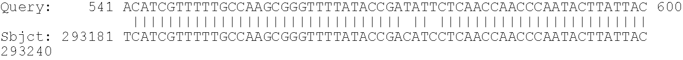

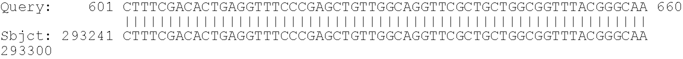

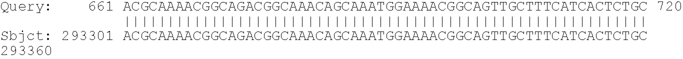

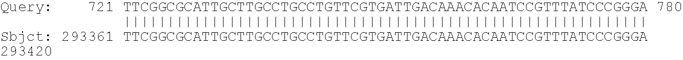

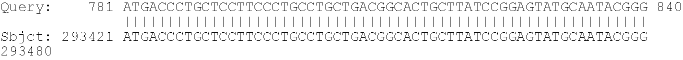

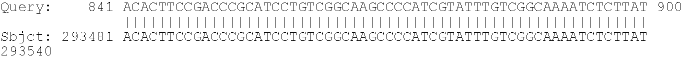

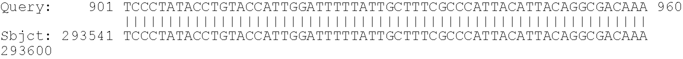

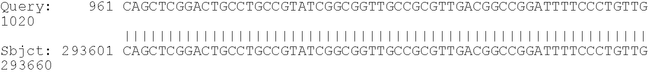

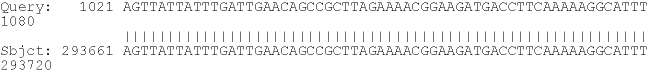

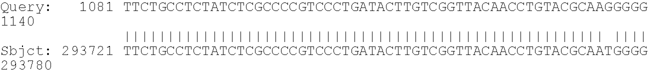

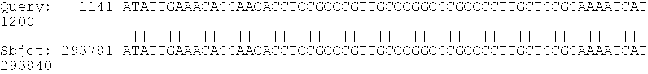

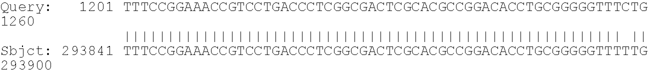

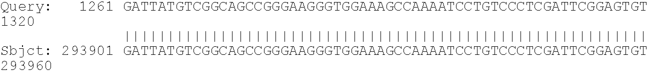

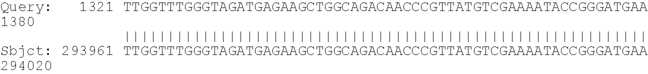

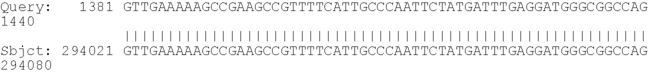

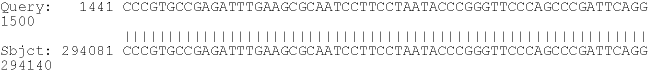

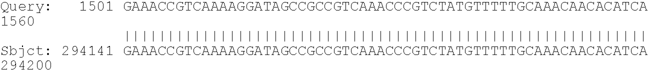

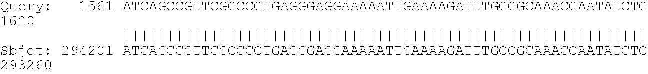

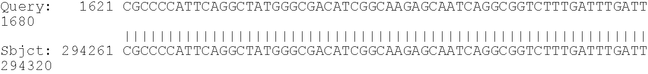

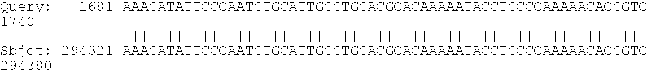

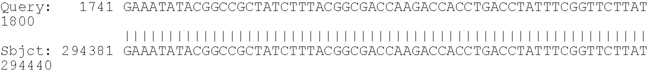

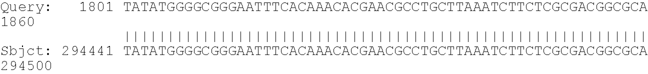

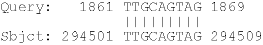

N. lactamica has not been assembled into genome. The position indicators below are for an assembled read. In addition, note this is the reverse complement of the lot gene.

TABLE 10
>lact221a11.p2kA115 373210 bp, 7825 reads, 47.57 AT (SEQ ID NO:34,
Query, SEQ ID NO:35, Subject)
Minus Strand HSPs:
Score = 8907 (1342.5 bits), Expect = 0., P = 0.
Identities = 1825/1869 (97%), Positives = 1825/1869 (97%), Strand = Minus/
Plus

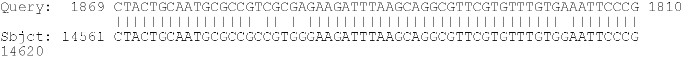

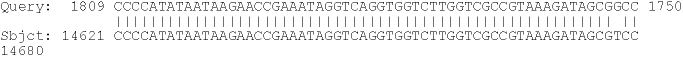

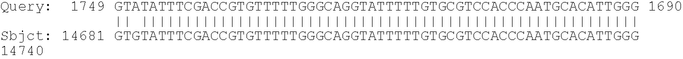

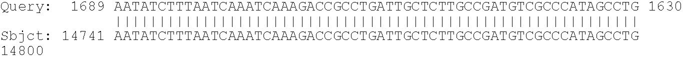

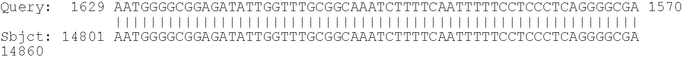

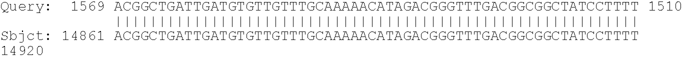

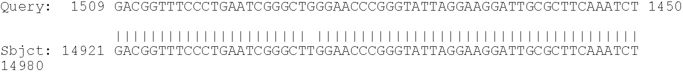

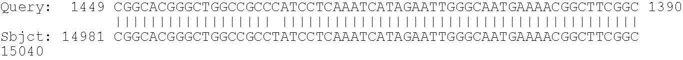

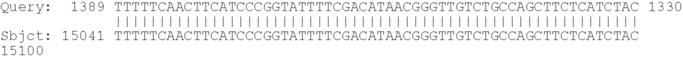

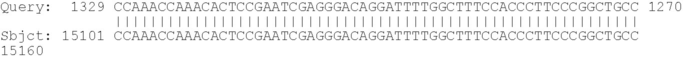

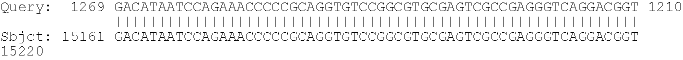

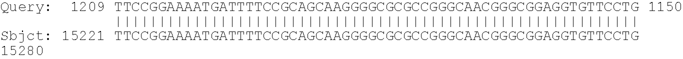

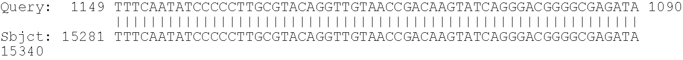

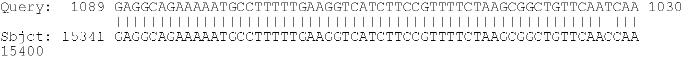

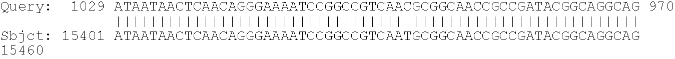

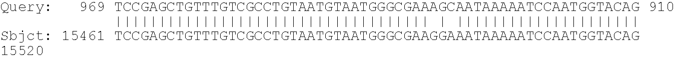

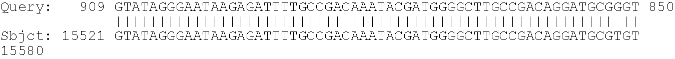

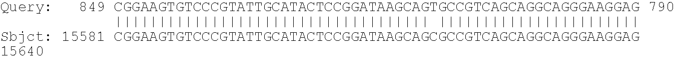

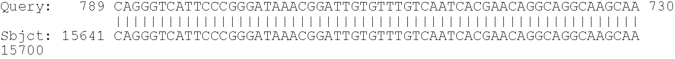

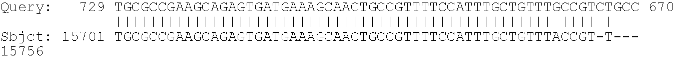

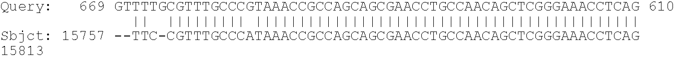

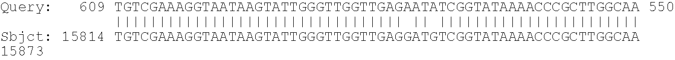

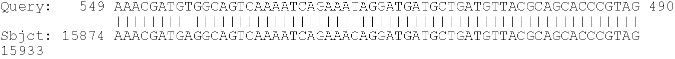

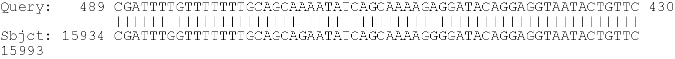

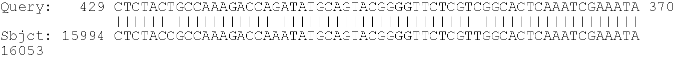

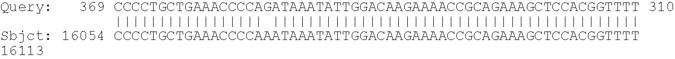

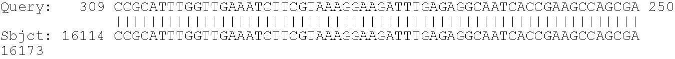

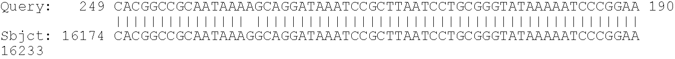

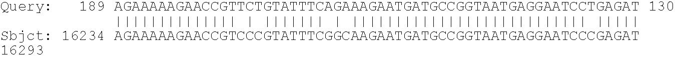

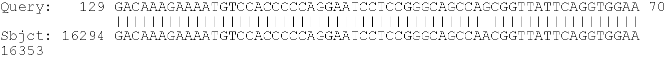

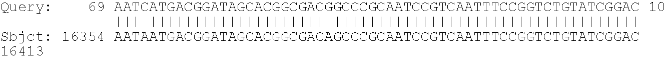

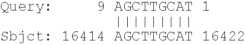

Without wishing to be bound by any particular theory, it is believed that the N. lactamica sequence provided above encoded a functional O-acetyl transferase enzyme with that equivalent to that of the N. meningitidis Lot3 protein exemplified herein.

TABLE 11
Immunotyping strains used in this study.

| | Strain which has been | | Strains used |
|---|---|---|---|
| Immunotype | chemically defined | Reference | in this study |
| L1 | 126E | (9) | 126E |
| L2 | Serogroup C 2241 | (10) | 35E |
| L3 | Serogroup B MC58 | (13) | 6275 |
| L4 | Serogroup C strain 89I | (11) | 89I |
| L5 | Serogroup B 981 | (12) | M981 |
| L6 | M992 | (9) | M992 |
| L7 | Serogroup C strain | (11) | 6155 |
| | M982B | | |
| L8 | | | M978 |
| L9 | Serogroup A | | 120M |
| L10 | Serogroup A | | 7880 |
| L11 | Serogroup A | | 7889 |
| L12 | Serogroup A | | 7897 |

### BIBLIOGRAPHY

1. Yazdankhah, S. P., and Caugant, D. A. (2004) J Med Microbiol 53, 821-832
2. Neal, K. R., Nguyen-van-Tam, J. S., Slack, R. C., Kaczmarski, E. B., White, A., and Ala'Aldeen, D. A. (1999) Epidemiol Infect 123, 507-509
3. Jones, D. M., Borrow, R., Fox, A. J., Gray, S., Cartwright, K. A., and Poolman, J. T. (1992) Microb Pathog 13, 219-224
4. Scholten, R. J., Kuipers, B., Valkenburg, H. A., Dankert, J., Zollinger, W. D., and Poolman, J. T. (1994) J Med Microbiol 41, 236-243
5. Moller, A. S., Bjerre, A., Brusletto, B., Joo, G. B., Brandtzaeg, P., and Kierulf, P. (2005) J Infect Dis 191, 768-775
6. Mandrell, R. E., and Zollinger, W. D. (1977) Infect Immun 16, 471-475
7. Tsai, C. M., Mocca, L. F., and Frasch, C. E. (1987) Infect Immun 55, 1652-1656
8. Zollinger, W. D., and Mandrell, R. E. (1977) Infect Immun 18, 424-433
9. Difabio, J. L., Michon, F., Brisson, J., and Jennings, H. J. (1990) Can J Chem 68, 1029-1034
10. Gamian, A., Beurret, M., Michon, F., Brisson, J. R., and Jennings, H. J. (1992) J Biol Chem 267, 922-925
11. Kogan, G., Uhrin, D., Brisson, J. R., and Jennings, H. J. (1997) Carbohydr Res 298, 191-199
12. Michon, F., Beurret, M., Gamian, A., Brisson, J. R., and Jennings, H. J. (1990) J Biol Chem 265, 7243-7247
13. Pavliak, V., Brisson, J. R., Michon, F., Uhrin, D., and Jennings, H. J. (1993) J Biol Chem 268, 14146-14152
14. Kahler, C. M., and Stephens, D. S. (1998) Crit. Rev Microbiol 24, 281-334
15. Jennings, M. P., Srikhanta, Y. N., Moxon, E. R., Kramer, M., Poolman, J. T., Kuipers, B., and van der Ley, P. (1999) Microbiology 145 (Pt 11), 3013-3021
16. Banerjee, A., Wang, R., Uljon, S, N., Rice, P. A., Gotschlich, E. C., and Stein, D. C. (1998) Proc Natl Acad Sci USA 95, 10872-10877
17. Mackinnon, F. G., Cox, A. D., Plested, J. S., Tang, C. M., Makepeace, K., Coull, P. A., Wright, J. C., Chalmers, R., Hood, D. W., Richards, J. C., and Moxon, E. R. (2002) Mol Microbiol 43, 931-943
18. Kahler, C. M., Datta, A., Tzeng, Y. L., Carlson, R. W., and Stephens, D. S. (2005) Glycobiology 15, 409-419
19. Ram, S., Cox, A. D., Wright, J. C., Vogel, U., Getzlaff, S., Boden, R., L1, J., Plested, J. S., Meri, S., Gulati, S., Stein, D. C., Richards, J. C., Moxon, E. R., and Rice, P. A. (2003) J Biol Chem 278, 50853-50862
20. D'Haeze, W., and Holsters, M. (2002) Glycobiology 12, 79R-105R
21. Yanisch-Perron, C., Vieira, J., and Messing, J. (1985) Gene 33, 103-119
22. Morse, S. A., and Bartenstein, L. (1974) Proc Soc Exp Biol Med 145, 1418-1421
23. Kahler, C. M., Carlson, R. W., Rahman, M. M., Martin, L. E., and Stephens, D. S. (1996) J Bacteriol 178, 1265-1273
24. Chung, C. T., Niemela, S. L., and Miller, R. H. (1989) Proc Natl Acad Sci U S A 86, 2172-2175
25. Chema, R., Sugawara, H., Koike, T., Lopez, R., Gibson, T. J., Higgins, D. G., and Thompson, J. D. (2003) Nucleic Acids Res 31, 3497-3500
26. Tatusova, T. A., and Madden, T. L. (1999) FEMS Microbiol Lett 174, 247-250
27. Takeshita, S., Sato, M., Toba, M., Masahashi, W., and Hashimoto-Gotoh, T. (1987) Gene 61, 63-74
28. Janik, A., Juni, E., and Heym, G. A. (1976) J Clin Microbiol 4, 71-81
29. Tzeng, Y. L., Datta, A., Ambrose, K., Lo, M., Davies, J. K., Carlson, R. W., Stephens, D. S., and Kahler, C. M. (2004) J Biol Chem 279, 35053-35062
30. Kahler, C. M., Carlson, R. W., Rahman, M. M., Martin, L. E., and Stephens, D. S. (1996) J Bacteriol 178, 6677-6684
31. York, W. S., Darvill, A. G., McNeil, M., Stevenson, T. T., and Albersheim, P. (1985) Methods in Enzymology 118, 3-40
32. Ciucanu, I., and Kerek, F. (1984) Carbohydrate Research 131, 209-217
33. Firmin, J. L., Wilson, K. E., Carlson, R. W., Davies, A. E., and Downie, J. A. (1993) Mol Microbiol 10, 351-360
34. Davis, E. O., Evans, I. J., and Johnston, A. W. (1988) Mol Gen Genet. 212, 531-535
35. Warren, M. J., Roddam, L. F., Power, P. M., Terry, T. D., and Jennings, M. P. (2004) FEMS Immunol Med Microbiol 41, 43-50
36. Rahman, M. M., Stephens, D. S., Kahler, C. M., Glushka, J., and Carlson, R. W. (1998) Carbohydr Res 307, 311-324
37. Monteiro, M. A., Fortuna-Nevin, M., Farley, J., and Pavliak, V. (2003) Carbohydr Res 338, 2905-2912
38. Lee, F. K., Stephens, D. S., Gibson, B. W., Engstrom, J. J., Zhou, D., and Apicella, M. A. (1995) Infect Immun 63, 2508-2515
39. Kim, M. L., and Slauch, J. M. (1999) FEMS Immunol Med Microbiol 26, 83-92
40. Harvey, H. A., Jennings, M. P., Campbell, C. A., Williams, R., and Apicella, M. A. (2001) Mol Microbiol 42, 659-672
41. Plant, L., Sundqvist, J., Zughaier, S., Lovkvist, L., Stephens, D. S., and Jonsson, A. B. (2006) Infect Immun 74, 1360-1367
42. Virji, M., Makepeace, K., Peak, I. R., Ferguson, D. J., Jennings, M. P., and Moxon, E. R. (1995) Mol Microbiol 18, 741-754
43. Jennings, H. J., Lugowski, C., and Ashton, F. E. (1984) Infect Immun 43, 407-412
44. Verheul, A. F., Boons, G. J., Van der Marel, G. A., Van Boom, J. H., Jennings, H. J., Snippe, H., Verhoef, J., Hoogerhout, P., and Poolman, J. T. (1991) Infect Immun 59, 3566-3573
45. Cox, A. D., Zou, W., Gidney, M. A., Lacelle, S., Plested, J. S., Makepeace, K., Wright, J. C., Coull, P. A., Moxon, E. R., and Richards, J. C. (2005) Vaccine 23, 5045-5054
46. Jarva, H., Ram, S., Vogel, U., Blom, A. M., and Meri, S. (2005) J Immunol 174, 6299-6307

1. A DNA molecule comprising a Neisseria lot 3-O-acetyltransferase protein coding sequence and a transcriptional control sequence with which said 3-O-acetyltransferase coding sequence is not associated in nature, said coding sequence and said transcriptional control sequence being operably linked.

2. The DNA molecule of claim 1, wherein said O-acetyltransferase protein comprises the amino acid sequence of any of SEQ ID NOs:5 to 16 or SEQ ID NO:30, or a sequence having at least 25%, at least 50%, at least 75%, at least 85%, at least 90%, at least 95% or at least 98% sequence identity to SEQ ID NO:5, and wherein said protein has enzymatic activity of a lipooligosaccharide 3-O-acetyltransferase.

3. The DNA molecule of claim 2, wherein said 3-O-acetyltransferase coding sequence encodes the amino acid sequence as given in any of SEQ ID NOs:32 or 34 or comprises an amino acid sequence with at least 25%, at least 50%, at least 75%, at least 85%, at least 90%, at least 95% or at least 98% sequence identity to the amino acid sequence set forth in SEQ ID NO:32.

4. The DNA molecule of claim 1, wherein the 3-O-acetyltransferase coding sequence comprises the sequence set forth in any of SEQ ID NO:17-32 or a sequence functionally equivalent to one of the foregoing.

5. The DNA molecule of claim 1, further comprising a sequence encoding an affinity tag, said sequence being fused in frame, selected from the group consisting of a streptavidin tag, a flagellar antigen epitope tag, a polyhistidine tag, a glutathione-S-transferase tag or a calmodulin tag or a streptactin tag.

6. The DNA molecule of claim 1, further comprising a vector sequence functional in a bacterial cell.

7. The DNA molecule of claim 6, wherein the vector sequence is functional in Escherichia coli.

8. The DNA molecule of claim 6, wherein the vector sequence is functional in Neisseria meningitidis or Neisseria gonorrhoeae.

9. A bacterial host cell in which the DNA molecule of claim 1 is stably maintained.

10. A method for recombinantly producing an O-acetyltransferase comprising the step of culturing the bacterial host cell of claim 9 under conditions such that the Lot3 O-acetyltransferase is expressed.

11. The method of claim 9 further comprising recovering the Lot3 O-acetyltransferase.

12. A method for acetylating lipooligosaccharide prepared from Neisseria meningitidis, said method comprising the step of contacting an isolated lipooligosaccharide with the 3-O-acetyltransferase produced by the method of claim 11.

13. An improved immunogenic composition comprising an acetylated lipooligosaccharide of Neisseria meningitidis, wherein the improvement comprises acetylation of the lipooligosaccharide according to the method of claim 12.

14. A lipooligosaccharide composition prepared from a lot3 mutant of Neisseria meningitidis.

15. A lipooligosaccharide composition prepared from a mutant of Neisseria meningitidis in which a lot3 and a Igt gene product are not functional.

16. A mutant of Neisseria meningitidis in which a lot3 gene product is not functional.

17. A mutant of Neisseria meningitidis in which a lot3 gene product and a Igt gene product are not functional.


SEQUENCE LISTING (38 sequences)

SEQ ID NO: 1 - DNA, 23 bp, Artificial
  Synthetic construct  oligonucleotide useful as a primer.
gtctcggtcgccgtaaagatagc

SEQ ID NO: 2 - DNA, 25 bp, Artificial
  Synthetic construct  oligonucleotide useful as a primer.
gcagtagaggaacagtattacctcc

SEQ ID NO: 3 - DNA, 23 bp, Artificial
  Synthetic construct  oligonucleotide useful as a primer.
cgatttgtcgcggaaagaaaccg

SEQ ID NO: 4 - DNA, 25 bp, Artificial
  Synthetic construct oligonucleotide useful as a primer.
gaagccaaagccaaattgcttgagc

SEQ ID NO: 5 - protein, 622 aa, Neisseria meningitidis
MQAVRYRPEIDGLRAVAVLSVIIFHLNNRWLPGGFLGVDIFFVISGFLITGIILSEIQNGSFSFRDFYTRRIKRIYPAFIAAVSLASVIASQIFLYEDFNQMRKTVELSAVFLSNIYLGFQQGYFDLSADENPVLHIWSLAVEEQYYLLYPLLLIFCCKKTKSLRVLRNISIILFLILTATSFLPSGFYTDILNQPNTYYLSTLRFPELLAGSLLAVYGQTQNGRRQTANGKRQLLSSLCFGALLACLFVIDKHNPFIPGMTLLLPCLLTALLIRSMQYGTLPTRILSASPIVFVGKISYSLYLYHWIFIAFAHYITGDKQLGLPAVSAVAALTAGFSLLSYYLIEQPLRKRKMTFKKAFFCLYLAPSLILVGYNLYARGILKQEHLRPLPGAPLAAENHFPETVLTLGDSHAGHLRGFLDYVGSREGWKAKILSLDSECLVWVDEKLADNPLCRKYRDEVEKAEAVFIAQFYDLRMGGQPVPRFEAQSFLIPGFPARFRETVKRIAAVKPVYVFANNTSISRSPLREEKLKRFAANQYLRPIQAMGDIGKSNQAVFDLVKDIPNVHWVDAQKYLPKNTVEIHGRYLYGDQDHLTYFGSYYMGREFHKHERLLKSSRDGALQ

SEQ ID NO: 6 - protein, 622 aa, Neisseria meningitidis
MQAVRYRPEIDGLRAVAVLSVIIFHLNNRWLPGGFLGVDIFFVISGFLITGIILSEIQNGSFSFRDFYTRRIKRIYPAFIAAVSLASVIASQIFLYEDFNQMRKTVELSAVFLSNIYLGFQQGYFDLSADENPVLHIWSLAVEEQYYLLYPLLLIFCCKKTKSLRVLRNISIILFLILTASSFLPSGFYTDILNQPNTYYLSTLRFPELLAGSLLAVYGQTQNGRRQTANGKRQLLSSLCFGALLACLFVIDKHNPFIPGMTLLLPCLLTALLIRSMQYGTLPTRILSASPIVFVGKISYSLYLYHWIFIAFAHYITGDKQLGLPAVSAVAALTAGFSLLSYYLIEQPLRKRKMTFKKAFFCLYLAPSLILVGYNLYARGILKQEHLRPLPGAPLAAENHFPETVLTLGDSHAGHLRGFLDYVGSREGWKAKILSLDSECLVWVDEKLADNPLCRKYRDEVEKAEAVFIAQFYDLRMGGQPVPRFEAQSFLIPGFQARFRETVKRIAAVKPVYVFANNTSISRSPLREEKLKRFAANQYLRPIQAMGDIGKSNQAVFDLVKDIPNVHWVDAQKYLPKNTVEIHGCYLYGDQDHLTYFGSYYMGREFHKHERLLKSSRDGALQ

SEQ ID NO: 7 - protein, 622 aa, Neisseria meningitidis
MQAVRYRPEIDGLRAVAVLSVIIFHLNNRWLPGGFLGVDIFFVISGFLITGIILSEIQNGSFSFRDFYTRRIKRIYPAFIAAVSLASVIASQIFLYEDFNQMRKTVELSAVFLSNIYLGFQQGYFDLSADENPVLHIWSLAVEEQYYLLYPLLLIFCCKKTKSLRVLRNISIILFLILTATSFLPSRFYTDILNQPNTYYLSTLRFPELLAGSLLAVYGQTQNGRRQTASGKRQLLSLLCFGALLACLFVIDKHNPFIPGMTLLLPCLLTALLIRSMQYGTLPTRILSASPIVFVGKISYSLYLYHWIFIAFAHYITGDKQLGLPAVSAVAALTAGFSLLSYYLIEQPLRKRKMTFKKAFFCLYLAPSLILVGYNLYARGILKQEHLRPLPGAPLAAENHFPETVLTLGDSHAGHLRGFLDYVGSREGWKAKILSLDSECLVWVDTTLADNPLCRKYRDEVEKAEAVFIAQFYDLRMGGQPVPRFEAQSFLIPGFQARFRETVKRIAAVKPVYVFANNTSISRSPLREEKLKRFAANQYLRPIQAMGDIGKSNQAVFDLVKDIPNVHWVDAQKYLPKNTVEIHGRYLYGDQDHLTYFGSYYMGREFHKHERLLKSSRGGALQ

SEQ ID NO: 8 - protein, 622 aa, Neisseria meningitidis; identical to SEQ ID NO: 6

SEQ ID NO: 9 - protein, 622 aa, Neisseria meningitidis
MQAVRYRPEIDGLRAVAVLSVMIFHLNDRWLPGGFLGVDIFFVISGFLITGIILSEIQNGSFSFRDFYTRRIKRIYPAFIAAVSLASVIASQIFLYEDFNQMRKTVELSAVFLSNIYLGFQQGYFDLSADENPVLHIWSLAVEEQYYLLYPLLLIFCCKKTKSLRVLRNISIILFLILTATSFLPSGFYTDILNQPNTYYLSTLRFPELLAGSLLAVYGQTQNGRRQTANGKRQLLSSLCFGALLACLFVIDKHNPFIPGMTLLLPCLLTALLIRSMQYGTLPTRILSASPIVFVGKISYSLYLYHWIFIAFAHYITGDKQLGLPAVSAVAALTAGFSLLSYYLIEQPLRKRKMTFKKAFFCLYLAPSLILVGYNLYARGILKQEHLRPLPGAPLAAENHFPETVLTLGDSHAGHLRGFLDYVGSREGWKAKILSLDSECLVWVDEKLADNPLCRKYRDEVEKAEAVFIAQFYDLRMGGQPVPRFEAQSFLIPGFPARFRETVKRIAAVKPVYVFANNTSISRSPLREEKLKRFAANQYLRPIQAMGDIGKSNQAVFDLIKDIPNVHWVDAQKYLPKNTVEIHGRYLYGDQDHLTYFGSYYMGREFHKHERLLKSSRGGALQ

SEQ ID NO: 10 - protein, 622 aa, Neisseria meningitidis; identical to SEQ ID NO: 6

SEQ ID NO: 11 - protein, 622 aa, Neisseria meningitidis
MQTVRYRPEIDGLRAVAVLSVIIFHLNNRWLPGGFLGVDIFFVISGFLITGIILSEIQNGSFSFRDFYTRRIKRIYPAFIAAVSLASVIASQIFLYEDFNQMRKTVELSAVFLSNIYLGFQQGYFDLSADENPVLHIWSLAVEEQYYLLYPLLLIFCCKKTKSLRVLCHISIILFLILTASSFLPSGFYTDILNQPNTYYLSTLRFPELLAGSLLAVYGQTQNGRRQTANGKRQLLSSLCFGALLACLFVIDKHNPFIPGMTLLLPCLLTALLIRSMQYGTLPTRILSASPIVFVGKISYSLYLYHWIFIAFAHYITGDKQLGLPAVSAVAALTAGFSLLSYYLIEQPLRKRKMTFKKAFFCLYLAPSLILVGYNLYARGILKQEHLRPLPGAPLAAENHFPETVLTLGDSHAGHLRGFLDYVGSREGWKAKILSLDSECLVWVDEKLADNPLCRKYRDEVEKAEAVFIAQFYDLRMGGQPVPRFEAQSFLIPGFPARFRETVKRIAAVKPVYVFANNTSISRSPLREEKLKRFAANQYLRPIQAMGDIGKSNQAVFDLIKDIPNVHWVDAQKYLPKNTVEIYGRYLYGDQDHLTYFGSYYMGREFHKHESLLKHSHGNALQ

SEQ ID NO: 12 - protein, 622 aa, Neisseria meningitidis
MQAVRYRPEIDGLRAVAVLSVIIFHLNNRWLPGGFLGVDIFFVISGFLITGIILSEIQNGSFSFRDFYTRRIKRIYPAFIAAVSLASVIASQIFLYEDFNQMRKTGEFSAVFLSNIYLGFQQGYFDLSADENPVLHIWSLAVEEQYYLLYPLLLIFCCKKTKSLRVLRNISIILFLILTATSFLPSGFYTDILNQPNTYYLSTLRFPELLAGSLLAVYGQTQNGRRQTANGKRQLLSSLCFGALLACLFVIDKHNPFIPGMTLLLPCLLTALLIRSMQYGTLPTRILSASSIVFVGKISYSLYLYHWIFIAFAHYITGDKQLGLPAVSAVAALTAGFSLLSYYLIEQPLRKRKMTFKKAFFCLYLAPSLILVGYNLYARGILKQEHLRPLPGAPLAAENHFPETVLTLGDSHAGHLRGFLDYVGSREGWKAKILSLDSECLVWVDEKLADNPLCRKYRDEVEKAEAVFIAQFYDLRMGGQPVPRFEAQSFLIPGFQARFRETVKRIAAVKPVYVFANNTSISRSPLREEKLKRFAANQYLRPIQAMGDIGKSNQAVFDLIKDIPNVHWVDAQKYLPKNTVEIYGRYLYGDQDHLTYFGSYYMGREFHKHERLLKSSRDGALQ

SEQ ID NO: 13 - protein, 622 aa, Neisseria meningitidis
MQAVRYRPEIDGLRAVAVLSVMIFHLNNRWLPGGFLGVDIFFVISGFLITGIILSEIQNGSFSFRDFYTRRIKRIYPAFIAAVSLASVIASQIFLYEDFNQMRKTVELSAVFLSNIYLGFQQGYFDLSADENPVLHIWSLAVEEQYYLLYPLLLIFCCKKTKSLRVLRNISIILFLILTATSFLPSGFYTDILNQPNTYYLSTLRFPELLAGSLLAVYGQTQNGRRQTANGKRQLLSSLCFGALLACLFVIDKHNPFIPGMTLLLPCLLTALLIRSMQYGTLPTRILSASPIVFVGKISYSLYLYHWIFIAFAHYITGDKQLGLPAVSAVAALTAGFSLLSYYLIEQPLRKRKMTFKKAFFCLYLAPSLILVGYNLYARGILKQEHLRPLPGAPLAAENHFPETVLTLGDSHAGHLRGFLDYVGSREGWKAKILSLDSECLVWVDEKLADNPLCRKYRDEVEKAEAVFIAQFYDLRMGGQPVPRFEAQSFLIPGFPARFRETVKRIAAVKPVYVFANNTSISRSPLREEKLKRFAANQYLRPIQAMGDIGKSNQAVFDLIKDIPNVHWVDAQKYLPKNTVEIYGRYLYGDQDHLTYFGSYYMGREFHKHERLLKSSRDGALQ

SEQ ID NO: 14 - protein, 622 aa, Neisseria meningitidis; identical to SEQ ID NO: 13

SEQ ID NO: 15 - protein, 622 aa, Neisseria meningitidis; identical to SEQ ID NO: 13

SEQ ID NO: 16 - protein, 622 aa, Neisseria meningitidis; identical to SEQ ID NO: 13

SEQ ID NO: 17 - DNA, 2347 bp, Neisseria meningitidis
aaacggatttgagcgtttactgaaaccgatgccgtctgaacgcgcgttcagacggcatttttaagataacgggacatacggggcgatatttatgcaagctgtccgatacaggcctgaaattgacggattgcgggccgttgccgtgctatccgtcattattttccacctgaataaccgctggctgcccggagggtttttgggggtggacattttctttgtcatctcgggattcctcattaccggcatcattctttctgaaatacagaacggttctttttctttccgggatttttatacccgcaggattaagcggatttaccctgcctttattgcggccgtgtcgctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcagtagaggaacagtattacctcctgtatcctcttttgctgatattttgctgcaaaaaaacaaaatcgctacgggtgctgcgtaacatcagcatcatcctatttctgattttgactgccacatcgtttttgccaagcgggttttataccgatattctcaaccaacccaatacttattacctttcgacactgaggtttcccgagctgttggcaggttcgctgctggcggtttacgggcaaacgcaaaacggcagacggcaaacagcaaatggaaaacggcagttgctttcatcactctgcttcggcgcattgcttgcctgcctgttcgtgattgacaaacacaatccgtttatcccgggaatgaccctgctccttccctgcctgctgacggcactgcttatccggagtatgcaatacgggacacttccgacccgcatcctgtcggcaagccccatcgtatttgtcggcaaaatctcttattccctatacctgtaccattggatttttattgctttcgcccattacattacaggcgacaaacagctcggactgcctgccgtatcggcggttgccgcgttgacggccggattttccctgttgagttattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcggaaaatcattttccggaaaccgtcctgaccctcggcgactcgcacgccggacacctgcgggggtttctggattatgtcggcagccgggaagggtggaaagccaaaatcctgtccctcgattcggagtgtttggtttgggtagatgagaagctggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttcccagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttggttaaagatatccccaatgtgcattgggtggacgcacagaaatacctgcctaaaaacacggtcgaaatacacggccgctatctttacggcgaccaagaccacctgacctatttcggttcttattatatggggcgggaatttcacaaacacgaacgcctgcttaaatcttctcgcgacggcgcattgcagtagcctgccttgccgtccgatatcgtttgtgccgccgtttgcctttcggggcggcggcttttatagtggattaacaaaaatcaggacaaggcaacgaagccgcagacagtacaaatagtacggaaccgattcacttggtgcttcagcaccttagagaatcgttctctttgagctaaggcgaggcaacgccgtactggtttttgttaatccactatattttgccgttttgaggccggggtcggaataaccgttttttgatgattttccctccctggctgtgtcatcaaaaccccaattgcctttccaaactctccaccagattgtcatccagtttcaaagcctgcgacaggcgggcgaggaagacggtttctttccgggaacggaatcgaa

SEQ ID NO: 18 - DNA, 2332 bp, Neisseria meningitidis
aacggatttgagcgtttactgaaaccgatgccgtctgaacgcgcgttcagacggcatttttaagataacgggacatacggggcgatatttatgcaagctgtccgatacaggcctgaaattgacggattgcgggccgttgccgtgctatccgtcattattttccacctgaataaccgctggctgcccggagggtttttgggggtggacattttctttgtcatctcgggattcctcattaccggcatcattctttctgaaatacagaacggttctttttctttccgggatttttatacccgcaggattaagcggatttaccctgcctttattgcggccgtgtcgctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcggtagaggaacagtattacctcctgtatccccttttgctgatattttgctgcaaaaaaaccaaatcgctacgggtgctgcgtaacatcagcatcatcctgtttttgattttgactgcctcatcgtttttgccaagcgggttttataccgacatcctcaaccaacccaatacttattacctttcgacactgaggtttcccgagctgttggcaggttcgctgctggcggtttacgggcaaacgcaaaacggcagacggcaaacagcaaatggaaaacggcagttgctttcatcactctgcttcggcgcattgcttgcctgcctgttcgtgattgacaaacacaatccgtttatcccgggaatgaccctgctccttccctgcctgctgacggcactgcttatccggagtatgcaatacgggacacttccgacccgcatcctgtcggcaagccccatcgtatttgtcggcaaaatctcttattccctatacctgtaccattggatttttattgctttcgcccattacattacaggcgacaaacagctcggactgcctgccgtatcggcggttgccgcgttgacggccggattttccctgttgagttattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcggaaaatcattttccggaaaccgtcctgaccctcggcgactcgcacgccggacacctgcgggggtttctggattatgtcggcagccgggaagggtggaaagccaaaatcctgtccctcgattcggagtgtttggtttgggtagatgagaagctggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttccaagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttggttaaagatatccccaatgtgcattgggtggacgcacagaaatacctgcctaaaaacacggtcgaaatacacggctgctatctttacggcgaccaagaccacctgacctatttcggttcttattatatggggcgggaatttcacaaacacgaacgcctgcttaaatcttctcgcgacggcgcattgcagtagcctgccttgccgtccgatatcgtttgtgccgccgtttgcctttcggggcggcggcttttatagtggattaacaaaaatcaggacaaggcaacgaagccgcagacagtacaaatagtacggaaccgattcacttggtgcttcagcaccttagagaatcgttctctttgagctaaggcgaggcaacgccgtactggtttttgttaatccactatattttgccgttttgaggccggggtcggaataaccgttttttgatgattttccctccccggctgtgtcatcaaaaccccaattgcctttccaaactctccaccagattgtcatccagttccaaagcctgcgacaggcgggcgaggaagacggtttctttcgg

SEQ ID NO: 19 - DNA, 2368 bp, Neisseria meningitidis
cgcgagtggcagctgagcgttggcgaacggatttgagcgtttactgaaaccgatgccgtctgaacgcgcgttcagacggcatttttaagataacgggacatacgggggcgatatttatgcaagctgtccgatacaggcctgaaattgacggattgcgggccgttgccgtgctatccgtcattattttccacctgaataaccgctggctgcccggagggtttttgggggtggacattttctttgtcatctcgggattcctcattaccggcatcattctttctgaaatacagaacggttctttttctttccgggatttttatacccgcaggattaagcggatttatcctgcttttatcgcggccgtgtcgctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcggtagaggaacagtattacctcctgtatcctcttttgctgatattttgctgcaaaaaaaccaaatcgctacgggtgctgcgtaacatcagcatcatcctatttctgattttgactgccacatcgtttttgccaagcaggttttataccgacatcctcaaccaacccaatacttattacctttcgacactgaggtttcccgagctgttggcaggttcgctgctggcggtttacgggcaaacgcaaaacggcagacggcaaacagcaagcggaaaacggcagttgctttcattactctgcttcggcgcattgcttgcctgcctgttcgtgattgacaaacacaatccgtttatcccgggaatgaccctgctccttccctgcctgctgacggcactgcttatccggagtatgcaatacgggacacttccgacccgcatcctgtcggcaagccccatcgtatttgtcggcaaaatctcttattccctatacctgtaccattggatttttattgccttcgcccattacattacaggcgacaaacagctcggactgcctgccgtatcggcggttgccgcattgacggccggattttccctgttgagctattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcggaaaatcattttccggaaaccgtcctgaccctcggcgactcgcacgccggacacctgcggggttttctggattatgtcggcagccgggaagggtggaaagccaaaatcctgtccctcgattcggagtgtttggtttgggtggatacgacactggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttccaagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttggttaaagatatccccaatgtgcattgggtggacgcacagaaatacctgcctaaaaacacggtcgaaatacacggacgctatctttacggcgaccaagaccacctgacctatttcggttcttattatatggggcgggaatttcacaaacacgaacgcctgcttaaatcttcccgcggcggcgcattgcagtagcctgccttcttgtcggatattgcctttggcagcctatgccgctgtttgcccttcggggcggcggcttttatagtggattaacaaaaatcaggacaaggcgacgaagccgcagacagtacaaatagtacggaaccgattcacttggtgcttcagcaccttagagaatcgttctctttgagctaaggcgaggcaacgccgtactggtttttgttaatccactatattttgccgttttgaggccggggtcggaataaccgttttttgatgattttccctccctggctgtgtcatcaaaaccccaattgcctttccaaactctccaccagattgtcatccagtttcaaagcctgcgacaggcgggcgaggaagacggtttctttcgg

SEQ ID NO: 20 - DNA, 2346 bp, Neisseria meningitidis
agcgttggcgaaacggatttgagcgtttactgaaaccgatgccgtctgaacgcgcgttcagacggcatttttaagataacgggacatacggggcgatatttatgcaagctgtccgatacaggcctgaaattgacggattgcgggccgttgccgtgctatccgtcattattttccacctgaataaccgctggctgcccggagggtttttgggggtggacattttctttgtcatctcgggattcctcattaccggcatcattctttctgaaatacagaacggttctttttctttccgggatttttatacccgcaggattaagcggatttaccctgcctttattgcggccgtgtcgctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcggtagaggaacagtattacctcctgtatccccttttgctgatattttgctgcaaaaaaaccaaatcgctacgggtgctgcgtaacatcagcatcatcctgtttttgattttgactgcctcatcgtttttgccaagcgggttttataccgacatcctcaaccaacccaatacttattacctttcgacactgaggtttcccgagctgttggcaggttcgctgctggcggtttacgggcaaacgcaaaacggcagacggcaaacagcaaatggaaaacggcagttgctttcatcactctgcttcggcgcattgcttgcctgcctgttcgtgattgacaaacacaatccgtttatcccgggaatgaccctgctccttccctgcctgctgacggcactgcttatccggagtatgcaatacgggacacttccgacccgcatcctgtcggcaagccccatcgtatttgtcggcaaaatctcttattccctatacctgtaccattggatttttattgctttcgcccattacattacaggcgacaaacagctcggactgcctgccgtatcggcggttgccgcgttgacggccggattttccctgttgagttattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcggaaaatcattttccggaaaccgtcctgaccctcggcgactcgcacgccggacacctgcgggggtttctggattatgtcggcagccgggaagggtggaaagccaaaatcctgtccctcgattcggagtgtttggtttgggtagatgagaagctggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttccaagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttggttaaagatatccccaatgtgcattgggtggacgcacagaaatacctgcctaaaaacacggtcgaaatacacggctgctatctttacggcgaccaagaccacctgacctatttcggttcttattatatggggcgggaatttcacaaacacgaacgcctgcttaaatcttctcgcgacggcgcattgcagtagcctgccttgccgtccgatatcgtttgtgccgccgtttgcctttcggggcggcggcttttatagtggattaacaaaaatcaggacaaggcaacgaagccgcagacagtacaaatagtacggaaccgattcacttggtgcttcagcaccttagagaatcgttctctttgagctaaggcgaggcaacgccgtactggtttttgttaatccactatattttgccgttttgaggccggggtcggaataaccgttttttgatgattttccctccccggctgtgtcatcaaaaccccaattgcctttccaaactctccaccagattgtcatccagttccaaagcctgcgacaggcgggcgaggaagacggtttctttcgggga

SEQ ID NO: 21 - DNA, 2203 bp, Neisseria meningitidis
acgcgagtgggcaggctgagcgttggcgaacggatttgagcgtttactgaaaccgatgccgtctgaacgcgcgttcagacggcatttttaagataacgggacatacgggggcgatatttatgcaagctgtccgatacaggcctgaaattgacggattgcgggccgtcgccgtgctatccgtcatgattttccacctgaatgaccgctggctgcccggaggattcctgggggtggacattttctttgtcatctcaggattcctcattaccggcatcattctttctgaaatacagaacggttctttttctttccgggatttttatacccgcaggattaagcggatttatcctgcttttattgcggccgtgtcgctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcagtagaggaacagtattacctcctgtatcctcttttgctgatattttgctgcaaaaaaacaaaatcgctacgggtgctgcgtaacatcagcatcatcctatttctgattttgactgccacatcgtttttgccaagcgggttttataccgatattctcaaccaacccaatacttattacctttcgacactgaggtttcccgagctgttggcaggttcgctgctggcggtttacgggcaaacgcaaaacggcagacggcaaacagcaaatggaaaacggcagttgctttcatcactctgcttcggcgcattgcttgcctgcctgttcgtgattgacaaacacaatccgtttatcccgggaatgaccctgctccttccctgcctgctgacggcactgcttatccggagtatgcaatacgggacacttccgacccgcatcctgtcggcaagccccatcgtatttgtcggcaaaatctcttattccctatacctgtaccattggatttttattgccttcgcccattacattacaggcgacaaacagctcggactgcctgccgtatcggcggttgccgcgttgacggccggattttccctgttgagttattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcagaaaatcattttccggaaaccgtcctgaccctcggcgactcgcacgccggacacctgcggggttttctggattatgtcggcagccgggaagggtggaaagccaaaatcctgtccctcgattcggagtgtttggtttgggtagatgagaagctggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttcccagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttgattaaagatattcccaatgtgcattgggtggacgcacaaaaatacctgcctaaaaacacggtcgaaatacacggccgctatctttacggcgaccaagaccacctgacctatttcggttcttattatatggggcgggaatttcacaaacacgaacgcctgcttaaatcttcccgcggcggcgcattgcagtagcctgccttcttgtcggatattgcctttggcagcctatgccgctgtttgccgttttgaggccggggtcggaataaccgttttttgatgattttccctccccggctgtgtcatcaaaaccccaattgcctttccaaactctccaccagattgtcatccagtttcaaagcctgcgacaggcgggcgaggaagacggtttctttccgcgaacaaatcga

SEQ ID NO: 22 - DNA, 2354 bp, Neisseria meningitidis
gggcaggctgagcgttggcgaaacggatttgagcgtttactgaaaccgatgccgtctgaacgcgcgttcagacggcatttttaagataacgggacatacggggcgatatttatgcaagctgtccgatacaggcctgaaattgacggattgcgggccgttgccgtgctatccgtcattattttccacctgaataaccgctggctgcccggagggtttttgggggtggacattttctttgtcatctcgggattcctcattaccggcatcattctttctgaaatacagaacggttctttttctttccgggatttttatacccgcaggattaagcggatttaccctgcctttattgcggccgtgtcgctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcggtagaggaacagtattacctcctgtatccccttttgctgatattttgctgcaaaaaaaccaaatcgctacgggtgctgcgtaacatcagcatcatcctgtttttgattttgactgcctcatcgtttttgccaagcgggttttataccgacatcctcaaccaacccaatacttattacctttcgacactgaggtttcccgagctgttggcaggttcgctgctggcggtttacgggcaaacgcaaaacggcagacggcaaacagcaaatggaaaacggcagttgctttcatcactctgcttcggcgcattgcttgcctgcctgttcgtgattgacaaacacaatccgtttatcccgggaatgaccctgctccttccctgcctgctgacggcactgcttatccggagtatgcaatacgggacacttccgacccgcatcctgtcggcaagccccatcgtatttgtcggcaaaatctcttattccctatacctgtaccattggatttttattgctttcgcccattacattacaggcgacaaacagctcggactgcctgccgtatcggcggttgccgcgttgacggccggattttccctgttgagttattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcggaaaatcattttccggaaaccgtcctgaccctcggcgactcgcacgccggacacctgcgggggtttctggattatgtcggcagccgggaagggtggaaagccaaaatcctgtccctcgattcggagtgtttggtttgggtagatgagaagctggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttccaagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttggttaaagatatccccaatgtgcattgggtggacgcacagaaatacctgcctaaaaacacggtcgaaatacacggctgctatctttacggcgaccaagaccacctgacctatttcggttcttattatatggggcgggaatttcacaaacacgaacgcctgcttaaatcttctcgcgacggcgcattgcagtagcctgccttgccgtccgatatcgtttgtgccgccgtttgcctttcggggcggcggcttttatagtggattaacaaaaatcaggacaaggcaacgaagccgcagacagtacaaatagtacggaaccgattcacttggtgcttcagcaccttagagaatcgttctctttgagctaaggcgaggcaacgccgtactggtttttgttaatccactatattttgccgttttgaggccggggtcggaataaccgttttttgatgattttccctccccggctgtgtcatcaaaaccccaattgcctttccaaactctccaccagattgtcatccagttccaaagcctgcgacaggcgggcgaggaagacggtttctttcggg

SEQ ID NO: 23 - DNA, 2363 bp, Neisseria meningitidis
aggggcagctgagcgttggcgaacggatttgagcgtttactgaaaccgatgccgtctggacgcgcgttcagacggcatttttaaaataccggatatacaggggcgatatttatgcaaactgtccgatacaggcctgaaattgacggattacgggctgtcgccgtcctttccgtcattattttccacctgaataaccgttggctgcccggaggattcctgggggtggacattttctttgtcatctcgggattcctcattaccggcatcattctttctgaaatacagaacggttctttttctttccgggatttttatacccgcaggattaagcggatttaccctgcctttattgcggccgtgtcgctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcggtagaggaacagtattacctcctgtatcctcttttgctgatattttgctgcaaaaaaacaaaatcgctacgggtgttgtgccacatcagcatcatcctgtttttgattttgactgcctcatcgtttttgccaagcgggttttataccgacatcctcaaccaacccaatacttattacctttcgacactgaggtttcccgagctgttggcaggttcgctgctggcggtttacgggcaaacgcaaaacggcagacggcaaacagcaaatggaaaacggcagttgctttcatcactctgcttcggcgcattgcttgcctgcctgttcgtgattgacaaacacaatccgtttatcccgggaatgaccctgctccttccctgcctgctgacggcgctgcttatccggagtatgcaatacgggacacttccgacccgcatcctgtcggcaagccccatcgtatttgtcggcaaaatctcttattccctatacctgtaccattggatttttattgccttcgcccattacattacaggcgacaaacagctcggactgcctgccgtatcggcggttgccgcgttgacggccggattttccctgttgagttattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcggaaaatcattttccggaaaccgtcctgaccctcggcgactcgcacgccggacacctgcgggggtttctggattatgtcggcagccgggaagggtggaaagccaaaatcctgtccctcgattcggagtgtttggtttgggtagatgagaagctggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttcccagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttgattaaagatattcccaatgtgcattgggtggacgcacaaaaatacctgcccaaaaacacggtcgaaatatacggccgctatctttacggcgaccaagaccacctgacctatttcggttcttattatatggggcgggaatttcacaaacatgaaagcttgctcaagcattcacacggcaacgcattgcagtagcctgccttcttgtcggatattgcctttggcagcctatgccgctgtttgcccttcggggcggcggcttttatagtggattaacaaaaatcaggacaaggcgacgaagccgcagacagtacaaatagtacggaaccgattcacttggtgcttcagcaccttagagaatcgttctctttgagctaaggcgaggcaacgccgtactggtttttgttaatccactatattttgccgttttgaggccggggtcggaataaccgttttttgatgattttccctccccggctgtgtcatcaaaaccccaattgcctttccaaactctccaccagattgtcatccagtttcaaagcctgcgacaggcgggcgaggaagacggtttctttcgg

SEQ ID NO: 24 - DNA, 1869 bp, Neisseria meningitidis
atgcaagctgtccgatacaggcctgaaattgacggattgcgggccgttgccgtgctatccgtcattattttccacctgaataaccgctggctgcccggagggtttttgggggtggacattttctttgtcatctcgggattcctcattaccggcatcattctttctgaaatacagaacggttctttttctttccgggatttttatacccgcaggattaagcggatttaccctgcctttattgcggccgtgtcgctggcttcggtgattgcctctcaaatcttcctttacgaagatttcaaccaaatgcggaaaaccggggagttttctgcggttttcttgtccaatatttatctggggtttcagcaggggtatttcgatttgagtgccgacgagaaccccgtactgcatatctggtctttggcagtagaggaacagtattacctcctgtatcctcttttgctgatattttgctgcaaaaaaacaaaatcgctacgggtgctgcgtaacatcagcatcatcctatttctgattttgactgccacatcgtttttgccaagcgggttttataccgatattctcaaccaacccaatacttattacctttcgacactgaggtttcccgagctgttggcaggttcgctgctggcggtttacgggcaaacgcaaaacggcagacggcaaacagcaaatggaaaacggcagttgctttcatcactctgcttcggcgcattgcttgcctgcctgttcgtgattgacaaacacaatccgtttatcccgggaatgaccctgctccttccctgcctgctgacggcgctgcttatccggagtatgcaatacgggacacttccgacccgcatcctgtcggcaagctccatcgtatttgtcggcaaaatctcttattccctatacctgtaccattggatttttattgctttcgcccattacattacaggcgacaaacagctcggactgcctgccgtatcggcggttgccgcgttgacggccggattttccctgttgagttattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcggaaaatcattttccggaaaccgtcctgaccctcggcgactcgcacgccggacacctgcgggggtttctggattatgtcggcagccgggaagggtggaaagccaaaatcctgtccctcgattcggagtgtttggtttgggtagatgagaagctggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttccaagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttgattaaagatattcccaatgtgcattgggtggacgcacaaaaatacctgcccaaaaacacggtcgaaatatacggccgctatctttacggcgaccaagaccacctgacctatttcggttcttattatatggggcgggaatttcacaaacacgaacgcctgcttaaatcttctcgcgacggcgcattgcagtag

SEQ ID NO: 25 - DNA, 1869 bp, Neisseria meningitidis
atgcaagctgtccgatacagaccggaaattgacggattgcgggccgtcgccgtgctatccgtcatgattttccacctgaataaccgctggctgcccggaggattcctgggggtggacattttctttgtcatctcaggattcctcattaccggcatcattctttctgaaatacagaacggttctttttctttccgggatttttatacccgcaggattaagcggatttatcctgcttttattgcggccgtgtcgctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcagtagaggaacagtattacctcctgtatcctcttttgctgatattttgctgcaaaaaaacaaaatcgctacgggtgctgcgtaacatcagcatcatcctatttctgattttgactgccacatcgtttttgccaagcgggttttataccgatattctcaaccaacccaatacttattacctttcgacactgaggtttcccgagctgttggcaggttcgctgctggcggtttacgggcaaacgcaaaacggcagacggcaaacagcaaatggaaaacggcagttgctttcatcactctgcttcggcgcattgcttgcctgcctgttcgtgattgacaaacacaatccgtttatcccgggaatgaccctgctccttccctgcctgctgacggcactgcttatccggagtatgcaatacgggacacttccgacccgcatcctgtcggcaagccccatcgtatttgtcggcaaaatctcttattccctatacctgtaccattggatttttattgctttcgcccattacattacaggcgacaaacagctcggactgcctgccgtatcggcggttgccgcgttgacggccggattttccctgttgagttattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcggaaaatcattttccggaaaccgtcctgaccctcggcgactcgcacgccggacacctgcgggggtttctggattatgtcggcagccgggaagggtggaaagccaaaatcctgtccctcgattcggagtgtttggtttgggtagatgagaagctggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttcccagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttgattaaagatattcccaatgtgcattgggtggacgcacaaaaatacctgcccaaaaacacggtcgaaatatacggccgctatctttacggcgaccaagaccacctgacctatttcggttcttattatatggggcgggaatttcacaaacacgaacgcctgcttaaatcttctcgcgacggcgcattgcagtag

SEQ ID NO: 26 - DNA, 2180 bp, Neisseria meningitidis
gggcaggctgagcgttggcgaaacggatttgagcgtttactgaaaccgatgccgtctgaacgcgcgttcagacggcatttttaagataacgggacatacaggggcgatatttatgcaagctgtccgatacagaccggaaattgacggattgcgggccgtcgccgtgctatccgtcatgattttccacctgaataaccgctggctgcccggaggattcctgggggtggacattttctttgtcatctcaggattcctcattaccggcatcattctttctgaaatacagaacggttctttttctttccgggatttttatacccgcaggattaagcggatttatcctgcttttattgcggccgtgtcgctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcagtagaggaacagtattacctcctgtatcctcttttgctgatattttgctgcaaaaaaacaaaatcgctacgggtgctgcgtaacatcagcatcatcctatttctgattttgactgccacatcgtttttgccaagcgggttttataccgatattctcaaccaacccaatacttattacctttcgacactgaggtttcccgagctgttggcaggttcgctgctggcggtttacgggcaaacgcaaaacggcagacggcaaacagcaaatggaaaacggcagttgctttcatcactctgcttcggcgcattgcttgcctgcctgttcgtgattgacaaacacaatccgtttatcccgggaatgaccctgctccttccctgcctgctgacggcactgcttatccggagtatgcaatacgggacacttccgacccgcatcctgtcggcaagccccatcgtatttgtcggcaaaatctcttattccctatacctgtaccattggatttttattgctttcgcccattacattacaggcgacaaacagctcggactgcctgccgtatcggcggttgccgcgttgacggccggattttccctgttgagttattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcagaaaatcattttccggaaaccgtcctgaccctcggcgactcgcacgccggacacctgcggggttttctggattatgtcggcagccgggaagggtggaaagccaaaatcctgtccctcgattcggagtgtttggtttgggtagatgagaagctggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttcccagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttgattaaagatattcccaatgtgcattgggtggacgcacaaaaatacctgcccaaaaacacggtcgaaatatacggccgctatctttacggcgaccaagaccacctgacctatttcggttcttattatatggggcgggaatttcacaaacacgaacgcctgcttaaatcttctcgcgacggcgcattgcagtagcctgccttgccgtccgatatcgtttgtgccgccgtttgcctttcggggcggcggcggtttttattttccttcccctgcgggagggaattttgaatcaaaaccccaattgcctttccaagttttccaccagattgtcatccagttccaaagcctgcgacaggcgggcgaggaagacggtttctttcccgaacaaatcgaa

SEQ ID NO: 27 - DNA, 2183 bp, Neisseria meningitidis
gtgggcaggctgagcgttggcgaaacggatttgagcgtttactgaaaccgatgccgtctgaacgcgcgttcagacggcatttttaagataacgggacatacaggggcgatatttatgcaagctgtccgatacagaccggaaattgacggattgcgggccgtcgccgtgctatccgtcatgattttccacctgaataaccgctggctgcccggaggattcctgggggtggacattttctttgtcatctcaggattcctcattaccggcatcattctttctgaaatacagaacggttctttttctttccgggatttttatacccgcaggattaagcggatttatcctgcttttattgcggccgtgtcgctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcagtagaggaacagtattacctcctgtatcctcttttgctgatattttgctgcaaaaaaacaaaatcgctacgggtgctgcgtaacatcagcatcatcctatttctgattttgactgccacatcgtttttgccaagcgggttttataccgatattctcaaccaacccaatacttattacctttcgacactgaggtttcccgagctgttggcaggttcgctgctggcggtttacgggcaaacgcaaaacggcagacggcaaacagcaaatggaaaacggcagttgctttcatcactctgcttcggcgcattgcttgcctgcctgttcgtgattgacaaacacaatccgtttatcccgggaatgaccctgctccttccctgcctgctgacggcactgcttatccggagtatgcaatacgggacacttccgacccgcatcctgtcggcaagccccatcgtatttgtcggcaaaatctcttattccctatacctgtaccattggatttttattgctttcgcccattacattacaggcgacaaacagctcggactgcctgccgtatcggcggttgccgcgttgacggccggattttccctgttgagttattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcggaaaatcattttccggaaaccgtcctgaccctcggcgactcgcacgccggacacctgcgggggtttctggattatgtcggcagccgggaagggtggaaagccaaaatcctgtccctcgattcggagtgtttggtttgggtagatgagaagctggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttcccagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttgattaaagatattcccaatgtgcattgggtggacgcacaaaaatacctgcccaaaaacacggtcgaaatatacggccgctatctttacggcgaccaagaccacctgacctatttcggttcttattatatggggcgggaatttcacaaacacgaacgcctgcttaaatcttctcgcgacggcgcattgcagtagcctgccttgccgtccgatatcgtttgtgccgccgtttgcctttcggggcggcggcggtttttattttccttcccctgcgggagggaattttgaatcaaaaccccaattgcctttccaagttttccaccagattgtcatccagttccaaagcctgcgacaggcgggcgaggaagacggtttctttccgcgaacaaatcgaa

SEQ ID NO: 28 - DNA, 1869 bp, Neisseria meningitidis; identical to SEQ ID NO: 25

SEQ ID NO: 29 - DNA, 1869 bp, Neisseria meningitidis; identical to SEQ ID NO: 25

SEQ ID NO: 30 - protein, 622 aa, Neisseria meningitidis; identical to SEQ ID NO: 13

SEQ ID NO: 31 - DNA, 1869 bp, Neisseria gonorrhoeae
atgcaagctgtccgatacaggcctgaaattgacggattgcgggccgtcgccgtgctatccgtcattattttccacctgaataaccgctggctgcccggaggattcctgggggtggacattttctttgtcatctcgggattcctcattaccaacatcattctttctgaaatacagaacggttctttttctttccgggatttttatacccgcaggattaagcggatttatcctgcttttattgcggccgtgtccctggcttcggtgattgcttctcaaatcttcctttacgaagatttcaaccaaatgaggaaaaccatagagctttctacggtttttttgtccaatatttatttggggttccgattggggtatttcgatttgagtgccgacgagaaccccgtactgcatatctggtctttggcggtagaggaacagtattacctcctgtatcctcttttgctgatattctgttacaaaaaaaccaaatcactacgggtgctgcgtaatatcagcatcatcctgtttctgattttgaccgcatcatcgtttttgccggccgggttttataccgacatcctcaaccaacccaatacttattacctttcgacactgaggtttcccgagctgttggtgggttcgctgttggcggtttacgggcaaacgcaaaacggcagacggcaaacagaaaatggaaaacggcagttgctttcattactctgtttcggcgcattgcttgtctgcctgttcgtgatcgacaaacacgatccgtttatcccgggaataaccctgctccttccctgcctgctgacggcgctgcttatccggagtatgcaatacgggacacttccgacccgcatcctgtcggcaagccccatcgtatttgtcggcaaaatctcttattccctatacctgtaccattggatttttattgccttcgcccattacattacaggcgacaaacagctcggactgcctgccgtatcggcggttgccgcgttgacggccggattttccctgttgagctattatttgattgaacagccgcttagaaaacggaagatgaccttcaaaaaggcatttttctgcctttatctcgccccgtccctgatgcttgtcggttacaacctgtattcaagagggatattgaaacaggaacacctccgcccgctgcccggcacgcccgttgctgcggaaaataattttccggaaaccgtcttgaccctcggcgactcgcacgccggacacctgcgggggtttctggattatgtcggcggcagggaagggtggaaagctaaaatcctgtccctcgattcggagtgtttggtttgggtggatgagaagctggcagacaacccgttgtgccgaaaataccgggatgaagttgaaaaagccgaagctgttttcattgcccaattctatgatttgaggatgggcggccagcccgtgccgagatttgaagcgcaatccttcctgatacccgggttcaaagcccgattcagggaaaccgtcaagaggatagccgccgtcaaacctgtatatgtttttgcaaacaatacatcaatcagccgttctcccttgagggaggaaaaattgaaaagatttgctataaaccaatacctccggcctattcgggctatgggcgacatcggcaagagcaatcaggcggtctttgatttggttaaagatattcccaatgtgcattgggtggacgcacaaaaatacctgcccaaaaacacggtcgaaatacacggacgctatctttacggcgaccaagaccacctgacctatttcggttcttattatatggggcgggaatttcacaaacacgaacgcctgctcaagcattcccgaggcggcgcattgcagtag

SEQ ID NO: 32 - protein, 622 aa, Neisseria gonorrhoeae
MQAVRYRPEIDGLRAVAVLSVIIFHLNNRWLPGGFLGVDIFFVISGFLITNIILSEIQNGSFSFRDFYTRRIKRIYPAFIAAVSLASVIASQIFLYEDFNQMRKTIELSTVFLSNIYLGFRLGYFDLSADENPVLHIWSLAVEEQYYLLYPLLLIFCYKKTKSLRVLRNISIILFLILTASSFLPAGFYTDILNQPNTYYLSTLRFPELLVGSLLAVYGQTQNGRRQTENGKRQLLSLLCFGALLVCLFVIDKHDPFIPGITLLLPCLLTALLIRSMQYGTLPTRILSASPIVFVGKISYSLYLYHWIFIAFAHYITGDKQLGLPAVSAVAALTAGFSLLSYYLIEQPLRKRKMTFKKAFFCLYLAPSLMLVGYNLYSRGILKQEHLRPLPGTPVAAENNFPETVLTLGDSHAGHLRGFLDYVGGREGWKAKILSLDSECLVWVDEKLADNPLCRKYRDEVEKAEAVFIAQFYDLRMGGQPVPRFEAQSFLIPGFKARFRETVKRIAAVKPVYVFANNTSISRSPLREEKLKRFAINQYLRPIRAMGDIGKSNQAVFDLVKDIPNVHWVDAQKYLPKNTVEIHGRYLYGDQDHLTYFGSYYMGREFHKHERLLKHSRGGALQ

SEQ ID NO: 33 - DNA, 1875 bp, Neisseria gonorrhoeae
atgagccaagccttaccctaccgcccggacatcgacacattgcgcgccgccgccgtcttgtccgtcatcgtgttccatatcgaaaaggattggctgccgggcgggtttctcggtgtcgatatattctttgtgatttcaggctttttgatgacggcgatcctccttcgcgaaatgtccggggggcgtttcttcctcaagacattttatatccgccgcatcaaacggattttgcccgcatttttcgccgtattggcggcaacgctggcaggcggcttctttttattcaccaaagatgatttctttcttttgtggaaatccgcgctgaccgccttgggtttcgcctccaacctgtattttgcaagggggaaggattatttcgatcccgcgcaggaagaaaagcccctgctgcacatctggtctttgtcggtcgaagaacaattttactttgtctttccgatattgctgttgcttgtcgcccgcaaaagcctgcgcgtacagttcggcttcctcgccgcattgtgcgccttaagccttgccgcttcctttatgccttccgcgctcgataaatattacctgccccacctgcgcgcctgcgaaatgctggtcggatcgctgaccgccgtgcggatgcggtaccggcaacagcggaatcccgccgtcgggaaacggtatgccgccgtcggcgcattgttttccgcgtgcatactgtccgcctgcctgtttgcctattcggaacaaaccgcctatttcccgggccccgccgctttgattccctgtctggctgttgccgcgctgatttatttcaaccattacgaacacccgcttaaaaaatttttccaatggaaaatcaccgttgccgccggtttgatttcctattcgctttatctgtggcattggccgatattggcctttatgcgctatatcggcccggacaacctgccgccttattcgccggcggcagcgatcgtcctgaccctggcgttttccctgatttcttatcactgcatcgaaaagccgtttaaaaaatggaaaggctcgttcgcacaatccgttttatggatttatgccttgcctatgctcgttttgggcgcgggctcgtttttcgcgatgaggctgccgtttatggcgcaatacgaccgcttggggctgacgcgttccaacacctcctgccacaacaataccggcaaacaatgcctgtggggggatacggaaaaacagccggaactgctggttttgggcgactcccacgccgaccattacaaaacattcttcgatgccgtgggcaaaaaagaaaaatggtccgccactatggtttccgccgacgcctgcgcctatgtggaaggctacgcgtcccgtgtgttccaaaactgggccgcctgccgcgccgtttaccgctatgccgaagaacacctgccccggtatccgaaagtggttttggcgatgcgctggggcagccagatgcccgaaaacagccgctcccttgcctacgatgccggttttttccaaaaattcgaccgtatgctgcacaaactctcatccgaaaaacaagccgtttacctgatggcggacaacttggcttcgtcttacaacgtccagcgcgcctatatcttgtcttcacgcataccgggttgccgccaaacactgcgcccggacgacgaaagcaccctgaaagccaatgcccgcatcagggaattggcagccaaataccccaacgtctatattattgatgccgccgcctatatccccgcagatttccaaatcggcggattgccggtttactcggacaaagaccacatcaacccttacggcggcacagaattggcgaagcgtttttccgaaaaacaaaggtttctcgatacgcgccataaccattga

SEQ ID NO: 34 - protein, 624 aa, Neisseria gonorrhoeae
MSQALPYRPDIDTLRAAAVLSVIVFHIEKDWLPGGFLGVDIFFVISGFLMTAILLREMSGGRFFLKTFYIRRIKRILPAFFAVLAATLAGGFFLFTKDDFFLLWKSALTALGFASNLYFARGKDYFDPAQEEKPLLHIWSLSVEEQFYFVFPILLLLVARKSLRVQFGFLAALCALSLAASFMPSALDKYYLPHLRACEMLVGSLTAVRMRYRQQRNPAVGKRYAAVGALFSACILSACLFAYSEQTAYFPGPAALIPCLAVAALIYFNHYEHPLKKFFQWKITVAAGLISYSLYLWHWPILAFMRYIGPDNLPPYSPAAAIVLTLAFSLISYHCIEKPFKKWKGSFAQSVLWIYALPMLVLGAGSFFAMRLPFMAQYDRLGLTRSNTSCHNNTGKQCLWGDTEKQPELLVLGDSHADHYKTFFDAVGKKEKWSATMVSADACAYVEGYASRVFQNWAACRAVYRYAEEHLPRYPKVVLAMRWGSQMPENSRSLAYDAGFFQKFDRMLHKLSSEKQAVYLMADNLASSYNVQRAYILSSRIPGCRQTLRPDDESTLKANARIRELAAKYPNVYIIDAAAYIPADFQIGGLPVYSDKDHINPYGGTELAKRFSEKQRFLDTRHNH

SEQ ID NO: 35 - DNA, 1869 bp, Neisseria meningitidis
ctactgcaatgcgccgtcgcgagaagatttaagcaggcgttcgtgtttgtgaaattcccgccccatataataagaaccgaaataggtcaggtggtcttggtcgccgtaaagatagcggccgtatatttcgaccgtgtttttgggcaggtatttttgtgcgtccacccaatgcacattgggaatatctttaatcaaatcaaagaccgcctgattgctcttgccgatgtcgcccatagcctgaatggggcggagatattggtttgcggcaaatcttttcaatttttcctccctcaggggcgaacggctgattgatgtgttgtttgcaaaaacatagacgggtttgacggcggctatccttttgacggtttccctgaatcgggctgggaacccgggtattaggaaggattgcgcttcaaatctcggcacgggctggccgcccatcctcaaatcatagaattgggcaatgaaaacggcttcggctttttcaacttcatcccggtattttcgacataacgggttgtctgccagcttctcatctacccaaaccaaacactccgaatcgagggacaggattttggctttccacccttcccggctgccgacataatccagaaacccccgcaggtgtccggcgtgcgagtcgccgagggtcaggacggtttccggaaaatgattttccgcagcaaggggcgcgccgggcaacgggcggaggtgttcctgtttcaatatcccccttgcgtacaggttgtaaccgacaagtatcagggacggggcgagatagaggcagaaaaatgcctttttgaaggtcatcttccgttttctaagcggctgttcaatcaaataataactcaacagggaaaatccggccgtcaacgcggcaaccgccgatacggcaggcagtccgagctgtttgtcgcctgtaatgtaatgggcgaaagcaataaaaatccaatggtacaggtatagggaataagagattttgccgacaaatacgatggggcttgccgacaggatgcgggtcggaagtgtcccgtattgcatactccggataagcagtgccgtcagcaggcagggaaggagcagggtcattcccgggataaacggattgtgtttgtcaatcacgaacaggcaggcaagcaatgcgccgaagcagagtgatgaaagcaactgccgttttccatttgctgtttgccgtctgccgttttgcgtttgcccgtaaaccgccagcagcgaacctgccaacagctcgggaaacctcagtgtcgaaaggtaataagtattgggttggttgagaatatcggtataaaacccgcttggcaaaaacgatgtggcagtcaaaatcagaaataggatgatgctgatgttacgcagcacccgtagcgattttgtttttttgcagcaaaatatcagcaaaagaggatacaggaggtaatactgttcctctactgccaaagaccagatatgcagtacggggttctcgtcggcactcaaatcgaaatacccctgctgaaaccccagataaatattggacaagaaaaccgcagaaagctccacggttttccgcatttggttgaaatcttcgtaaaggaagatttgagaggcaatcaccgaagccagcgacacggccgcaataaaagcaggataaatccgcttaatcctgcgggtataaaaatcccggaaagaaaaagaaccgttctgtatttcagaaagaatgatgccggtaatgaggaatcctgagatgacaaagaaaatgtccacccccaggaatcctccgggcagccagcggttattcaggtggaaaatcatgacggatagcacggcgacggcccgcaatccgtcaatttccggtctgtatcggacagcttgcat

SEQ ID NO: 36 - DNA, 1862 bp, Neisseria lactamica
ctactgcaatgcgccgccgtgggaagatttaagcaggcgttcgtgtttgtggaattcccgccccatataataagaaccgaaataggtcaggtggtcttggtcgccgtaaagatagcgtccgtgtatttcgaccgtgtttttgggcaggtatttttgtgcgtccacccaatgcacattgggaatatctttaatcaaatcaaagaccgcctgattgctcttgccgatgtcgcccatagcctgaatggggcggagatattggtttgcggcaaatcttttcaatttttcctccctcaggggcgaacggctgattgatgtgttgtttgcaaaaacatagacgggtttgacggcggctatccttttgacggtttccctgaatcgggcttggaacccgggtattaggaaggattgcgcttcaaatctcggcacgggctggccgcctatcctcaaatcatagaattgggcaatgaaaacggcttcggctttttcaacttcatcccggtattttcgacataacgggttgtctgccagcttctcatctacccaaaccaaacactccgaatcgagggacaggattttggctttccacccttcccggctgccgacataatccagaaacccccgcaggtgtccggcgtgcgagtcgccgagggtcaggacggtttccggaaaatgattttccgcagcaaggggcgcgccgggcaacgggcggaggtgttcctgtttcaatatcccccttgcgtacaggttgtaaccgacaagtatcagggacggggcgagatagaggcagaaaaatgcctttttgaaggtcatcttccgttttctaagcggctgttcaaccaaataataactcaacagggaaaatccggccgtcaatgcggcaaccgccgatacggcaggcagtccgagctgtttgtcgcctgtaatgtaatgggcgaaggaaataaaaatccaatggtacaggtatagggaataagagattttgccgacaaatacgatggggcttgccgacaggatgcgtgtcggaagtgtcccgtattgcatactccggataagcagcgccgtcagcaggcagggaaggagcagggtcattcccgggataaacggattgtgtttgtcaatcacgaacaggcaggcaagcaatgcgccgaagcagagtgatgaaagcaactgccgttttccatttgctgtttaccgttttccgtttgcccataaaccgccagcagcgaacctgccaacagctcgggaaacctcagtgtcgaaaggtaataagtattgggttggttgaggatgtcggtataaaacccgcttggcaaaaacgatgaggcagtcaaaatcagaaacaggatgatgctgatgttacgcagcacccgtagcgatttggtttttttgcagcagaatatcagcaaaaggggatacaggaggtaatactgttcctctaccgccaaagaccaaatatgcagtacggggttctcgttggcactcaaatcgaaatacccctgctgaaaccccaaataaatattggacaagaaaaccgcagaaagctccacggttttccgcatttggttgaaatcttcgtaaaggaagatttgagaggcaatcaccgaagccagcgacacggccgcaataaaggcaggataaatccgcttaatcctgcgggtataaaaatcccggaaagaaaaagaaccgtcccgtatttcggcaagaatgatgccggtaatgaggaatcccgagatgacaaagaaaatgtccacccccaggaatcctccgggcagccaacggttattcaggtggaaaataatgacggatagcacggcgacagcccgcaatccgtcaatttccggtctgtatcggacagcttgcat

SEQ ID NO: 37 - DNA, 1868 bp, Neisseria meningitidis
atgcaagctgtccgatacagaccggaaattgacggattgcgggccgtcgccgtgctatccgtcatgattttccacctgaataaccgctggctgcccggaggattcctgggggtggacattttctttgtcatctcaggattcctcattaccggcatcattctttctgaaatacagaacggttctttttctttccgggatttttatacccgcaggattaagcggatttatcctgcttttattgcggccgtgtcgctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcagtagaggaacagtattacctcctgtatcctcttttgctgatattttgctgcaaaaaaacaaaatcgctacgggtgctgcgtaacatcagcatcatcctatttctgattttgactgccacatcgtttttgccaagcgggttttataccgatattctcaaccaacccaatacttattacctttcgacactgaggtttcccgagctgttggcaggttcgctgctggcggtttacgggcaaacgcaaaacggcagacggcaaacagcaaatggaaaacggcagttgctttcatcactctgcttcggcgcattgcttgcctgcctgttcgtgattgacaaacacaatccgtttatcccgggaatgaccctgctccttccctgcctgctgacggcactgcttatccggagtatgcaatacgggacacttccgacccgcatcctgtcggcaagccccatcgtatttgtcggcaaaatctcttatccctatacctgtaccattggatttttattgctttcgcccattacattacaggcgacaaacagctcggactgcctgccgtatcggcggttgccgcgttgacggccggattttccctgttgagttattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcggaaaatcattttccggaaaccgtcctgaccctcggcgactcgcacgccggacacctgcgggggtttctggattatgtcggcagccgggaagggtggaaagccaaaatcctgtccctcgattcggagtgtttggtttgggtagatgagaagctggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttcccagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttgattaaagatattcccaatgtgcattgggtggacgcacaaaaatacctgcccaaaaacacggtcgaaatatacggccgctatctttacggcgaccaagaccacctgacctatttcggttcttattatatggggcgggaatttcacaaacacgaacgcctgcttaaatcttctcgcgacggcgcattgcagtag

SEQ ID NO: 38 - DNA, 1869 bp, Neisseria meningitidis
atgcaagctgtccgatacaggcctgaaattgacggattgcgggccgtcgccgtgctatccgtcatgattttccacctgaatgaccgctggctgcccggagggtttttgggggtggacattttctttgtcatctcgggattcctcattaccggcatcattctttctgaaatacagaacggttctttttctttccggggtttttatacccgcaggattaagcggatttaccctgcctttattgcggctgtgtccctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcggtagaggaacagtattacctcctgtatcctcttttgctgatattttgctgcaaaaaaaccaaatcgctacgggtgttgtgccacatcagcatcatcctgtttttgattttgactgcctcatcgtttttgccaagcgggttttataccgacatcctcaaccaacccaatacttattacctttcgacactgaggtttcccgagctgttggcaggttcgctgctggcggtttacgggcaaacgcaaaacggcagacggcaaacagcaaatggaaaacggcagttgctttcatcactctgcttcggcgcattgcttgcctgcctgttcgtgattgacaaacacaatccgtttatcccgggaatgaccctgctccttccctgcctgctgacggcactgcttatccggagtatgcaatacgggacacttccgacccgcatcctgtcggcaagccccatcgtatttgtcggcaaaatctcttattccctatacctgtaccattggatttttattgctttcgcccattacattacaggcgacaaacagctcggactgcctgccgtatcggcggttgccgcgttgacggccggattttccctgttgagttattatttgattgaacagccgcttagaaaacggaagatgaccttcaaaaaggcatttttctgcctctatctcgccccgtccctgatacttgtcggttacaacctgtacgcaatggggatattgaaacaggaacacctccgcccgttgcccggcgcgccccttgctgcggaaaatcattttccggaaaccgtcctgaccctcggcgactcgcacgccggacacctgcgggggtttttggattatgtcggcagccgggaagggtggaaagccaaaatcctgtccctcgattcggagtgtttggtttgggtagatgagaagctggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttcccagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttgattaaagatattcccaatgtgcattgggtggacgcacaaaaatacctgcccaaaaacacggtcgaaatatacggccgctatctttacggcgaccaagaccacctgacctatttcggttcttattatatggggcgggaatttcacaaacacgaacgcctgcttaaatcttctcgcgacggcgcattgcagtag